SEQUENCE LISTING (608 sequences)

SEQ ID NO: 1 - protein, 175 aa, Homo sapiens
MSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 2 - protein, 89 aa, Homo sapiens
SSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 3 - protein, 85 aa, Homo sapiens
GKTPDAMKAAMALEKKLNQALLDLHALGSARTDPHLCDFLETHFLDEEVKLIKKMGDHLTNLHRLGGPEAGLGEYLFERLTLRHD

SEQ ID NO: 4 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 524 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 4 aa, synthetic construct
GGGS

SEQ ID NO: 7 - protein, 5 aa, synthetic construct
GGGGS

SEQ ID NO: 8 - protein, 122 aa, synthetic construct
EVQLVESGGGLVQPGGSLRLSCAASGFSFSANYYPCWVRQAPGKGLEWIGCIYGGSSDITYDANWTKGRFTISRDNSKNTVYLQMNSLRAEDTAVYYCARSAWYSGWGGDLWGQGTLVTVSS

SEQ ID NO: 9 - protein, 110 aa, synthetic construct
DIQMTQSPSSLSASVGDRVTITCQASQSISNRLAWYQQKPGKAPKLLIYSASTLASGVPSRFSGSGSGTDFTLTISSLQPEDFATYYCQSTYYGNDGNAFGTGTKVTVLG

SEQ ID NO: 10 - protein, 6 aa, synthetic construct
ANYYPC

SEQ ID NO: 11 - protein, 18 aa, synthetic construct
CIYGGSSDITYDANWTKG

SEQ ID NO: 12 - protein, 11 aa, synthetic construct
SAWYSGWGGDL

SEQ ID NO: 13 - protein, 11 aa, synthetic construct
QASQSISNRLA

SEQ ID NO: 14 - protein, 7 aa, synthetic construct
SASTLAS

SEQ ID NO: 15 - protein, 11 aa, synthetic construct
QSTYYGNDGNA

SEQ ID NO: 16 - protein, 9 aa, synthetic construct
GFSFSANYY

SEQ ID NO: 17 - protein, 9 aa, synthetic construct
IYGGSSDIT

SEQ ID NO: 18 - protein, 13 aa, synthetic construct
ARSAWYSGWGGDL

SEQ ID NO: 19 - protein, 6 aa, synthetic construct
QSISNR

SEQ ID NO: 20 - protein, 11 aa, synthetic construct
QSTYYGNDGNA

SEQ ID NO: 21 - protein, 8 aa, synthetic construct
GFSFSANY

SEQ ID NO: 22 - protein, 7 aa, synthetic construct
YGGSSDI

SEQ ID NO: 23 - protein, 11 aa, synthetic construct
SAWYSGWGGDL

SEQ ID NO: 24 - protein, 11 aa, synthetic construct
QASQSISNRLA

SEQ ID NO: 25 - protein, 7 aa, synthetic construct
SASTLAS

SEQ ID NO: 26 - protein, 11 aa, synthetic construct
QSTYYGNDGNA

SEQ ID NO: 27 - protein, 122 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDLMTTAPGTYFDLWGRGTLVTVSS

SEQ ID NO: 28 - protein, 107 aa, synthetic construct
AIRMTQSPPSLSASVGDRVTITCQASQDIGNSLGWYQQKPGKAPKLVIFDASDLETGVPSRFSGSGSGTDFSLTISSLQPEDFATYYCQQGNSFPLTFGQGTRLEIK

SEQ ID NO: 29 - protein, 5 aa, synthetic construct
SYAIS

SEQ ID NO: 30 - protein, 17 aa, synthetic construct
GIIPIFGTANYAQKFQG

SEQ ID NO: 31 - protein, 13 aa, synthetic construct
DLMTTAPGTYFDL

SEQ ID NO: 32 - protein, 11 aa, synthetic construct
QASQDIGNSLG

SEQ ID NO: 33 - protein, 7 aa, synthetic construct
DASDLET

SEQ ID NO: 34 - protein, 9 aa, synthetic construct
QQGNSFPLT

SEQ ID NO: 35 - protein, 8 aa, synthetic construct
GGTFSSYA

SEQ ID NO: 36 - protein, 8 aa, synthetic construct
IIPIFGTA

SEQ ID NO: 37 - protein, 15 aa, synthetic construct
ARDLMTTAPGTYFDL

SEQ ID NO: 38 - protein, 6 aa, synthetic construct
QDIGNS

SEQ ID NO: 39 - protein, 9 aa, synthetic construct
QQGNSFPLT

SEQ ID NO: 40 - protein, 7 aa, synthetic construct
GGTFSSY

SEQ ID NO: 41 - protein, 6 aa, synthetic construct
IPIFGT

SEQ ID NO: 42 - protein, 13 aa, synthetic construct
DLMTTAPGTYFDL

SEQ ID NO: 43 - protein, 11 aa, synthetic construct
QASQDIGNSLG

SEQ ID NO: 44 - protein, 7 aa, synthetic construct
DASDLET

SEQ ID NO: 45 - protein, 9 aa, synthetic construct
QQGNSFPLT

SEQ ID NO: 46 - protein, 121 aa, synthetic construct
EVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDGQRNSMREFDYWGQGTLVTVSS

SEQ ID NO: 47 - protein, 110 aa, synthetic construct
QSVLTQPPSASGTPGRRVTISCSGSSSNIGNNYVTWYQQLPGTAPKLLIYADSHRPSGVPDRFSGSKSGTSASLAISGLRSEDEADYYCATWDYSLSGYVFGCGTKLTVL

SEQ ID NO: 48 - protein, 5 aa, synthetic construct
SYDMS

SEQ ID NO: 49 - protein, 17 aa, synthetic construct
WISYSGGSIYYADSVKG

SEQ ID NO: 50 - protein, 12 aa, synthetic construct
DGQRNSMREFDY

SEQ ID NO: 51 - protein, 13 aa, synthetic construct
SGSSSNIGNNYVT

SEQ ID NO: 52 - protein, 7 aa, synthetic construct
ADSHRPS

SEQ ID NO: 53 - protein, 11 aa, synthetic construct
ATWDYSLSGYV

SEQ ID NO: 54 - protein, 8 aa, synthetic construct
GFTFSSYD

SEQ ID NO: 55 - protein, 8 aa, synthetic construct
ISYSGGSI

SEQ ID NO: 56 - protein, 14 aa, synthetic construct
ARDGQRNSMREFDY

SEQ ID NO: 57 - protein, 8 aa, synthetic construct
SSNIGNNY

SEQ ID NO: 58 - protein, 11 aa, synthetic construct
ATWDYSLSGYV

SEQ ID NO: 59 - protein, 7 aa, synthetic construct
GFTFSSY

SEQ ID NO: 60 - protein, 6 aa, synthetic construct
SYSGGS

SEQ ID NO: 61 - protein, 12 aa, synthetic construct
DGQRNSMREFDY

SEQ ID NO: 62 - protein, 13 aa, synthetic construct
SGSSSNIGNNYVT

SEQ ID NO: 63 - protein, 7 aa, synthetic construct
ADSHRPS

SEQ ID NO: 64 - protein, 11 aa, synthetic construct
ATWDYSLSGYV

SEQ ID NO: 65 - protein, 127 aa, synthetic construct
EVQLLESGGGLVQPGGSLRLSCAASGFTFSGYDMSWVRQAPGKCLEWVSVIYPDDGNTYYADSVKGRFTISRDNSKNTLYLQMNSLRAEDAAVYYCAKHGGQKPTTKSSSAYGMDGWGQGTLVTVSS

SEQ ID NO: 66 - protein, 110 aa, synthetic construct; identical to SEQ ID NO: 47

SEQ ID NO: 67 - protein, 5 aa, synthetic construct
GYDMS

SEQ ID NO: 68 - protein, 17 aa, synthetic construct
VIYPDDGNTYYADSVKG

SEQ ID NO: 69 - protein, 18 aa, synthetic construct
HGGQKPTTKSSSAYGMDG

SEQ ID NO: 70 - protein, 13 aa, synthetic construct
SGSSSNIGNNYVT

SEQ ID NO: 71 - protein, 7 aa, synthetic construct
ADSHRPS

SEQ ID NO: 72 - protein, 11 aa, synthetic construct
ATWDYSLSGYV

SEQ ID NO: 73 - protein, 8 aa, synthetic construct
GFTFSGYD

SEQ ID NO: 74 - protein, 8 aa, synthetic construct
IYPDDGNT

SEQ ID NO: 75 - protein, 20 aa, synthetic construct
AKHGGQKPTTKSSSAYGMDG

SEQ ID NO: 76 - protein, 8 aa, synthetic construct
SSNIGNNY

SEQ ID NO: 77 - protein, 11 aa, synthetic construct
ATWDYSLSGYV

SEQ ID NO: 78 - protein, 7 aa, synthetic construct
GFTFSGY

SEQ ID NO: 79 - protein, 6 aa, synthetic construct
YPDDGN

SEQ ID NO: 80 - protein, 18 aa, synthetic construct
HGGQKPTTKSSSAYGMDG

SEQ ID NO: 81 - protein, 13 aa, synthetic construct
SGSSSNIGNNYVT

SEQ ID NO: 82 - protein, 7 aa, synthetic construct
ADSHRPS

SEQ ID NO: 83 - protein, 11 aa, synthetic construct
ATWDYSLSGYV

SEQ ID NO: 84 - protein, 121 aa, synthetic construct
EVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRNSMREFDYWGQGTLVTVSS

SEQ ID NO: 85 - protein, 110 aa, synthetic construct
QSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGCGTKLTVL

SEQ ID NO: 86 - protein, 5 aa, synthetic construct
SYDMS

SEQ ID NO: 87 - protein, 17 aa, synthetic construct
WISYSGGSIYYADSVKG

SEQ ID NO: 88 - protein, 12 aa, synthetic construct
DAQRNSMREFDY

SEQ ID NO: 89 - protein, 13 aa, synthetic construct
SGSSSNIGNNYVT

SEQ ID NO: 90 - protein, 7 aa, synthetic construct
ADSHRPS

SEQ ID NO: 91 - protein, 11 aa, synthetic construct
ATWDYSLSGYV

SEQ ID NO: 92 - protein, 8 aa, synthetic construct
GFTFSSYD

SEQ ID NO: 93 - protein, 8 aa, synthetic construct
ISYSGGSI

SEQ ID NO: 94 - protein, 14 aa, synthetic construct
ARDAQRNSMREFDY

SEQ ID NO: 95 - protein, 8 aa, synthetic construct
SSNIGNNY

SEQ ID NO: 96 - protein, 11 aa, synthetic construct
ATWDYSLSGYV

SEQ ID NO: 97 - protein, 7 aa, synthetic construct
GFTFSSY

SEQ ID NO: 98 - protein, 6 aa, synthetic construct
SYSGGS

SEQ ID NO: 99 - protein, 12 aa, synthetic construct
DAQRNSMREFDY

SEQ ID NO: 100 - protein, 13 aa, synthetic construct
SGSSSNIGNNYVT

SEQ ID NO: 101 - protein, 7 aa, synthetic construct
ADSHRPS

SEQ ID NO: 102 - protein, 11 aa, synthetic construct
ATWDYSLSGYV

SEQ ID NO: 103 - protein, 117 aa, synthetic construct
EVQLVESGGGLVQPGRSLRLSCTASGFSLNDYWMSWVRQAPGKGLEWVGYIDVGGSLYYAASVKGRFTISRDDSKSIAYLQMNSLKTEDTAVYYCARGGLTYGFDLWGQGTLVTVSS

SEQ ID NO: 104 - protein, 110 aa, synthetic construct
DIVMTQSPSSLSASVGDRVTITCQASEDISSYLAWYQQKPGKAPKRLIYGASDLASGVPSRFSASGSGTDYTFTISSLQPEDIATYYCHYYATISGLGVAFGGGTKVEIK

SEQ ID NO: 105 - protein, 5 aa, synthetic construct
DYWMS

SEQ ID NO: 106 - protein, 16 aa, synthetic construct
YIDVGGSLYYAASVKG

SEQ ID NO: 107 - protein, 9 aa, synthetic construct
GGLTYGFDL

SEQ ID NO: 108 - protein, 11 aa, synthetic construct
QASEDISSYLA

SEQ ID NO: 109 - protein, 7 aa, synthetic construct
GASDLAS

SEQ ID NO: 110 - protein, 12 aa, synthetic construct
HYYATISGLGVA

SEQ ID NO: 111 - protein, 8 aa, synthetic construct
GFSLNDYW

SEQ ID NO: 112 - protein, 7 aa, synthetic construct
IDVGGSL

SEQ ID NO: 113 - protein, 11 aa, synthetic construct
ARGGLTYGFDL

SEQ ID NO: 114 - protein, 6 aa, synthetic construct
EDISSY

SEQ ID NO: 115 - protein, 12 aa, synthetic construct
HYYATISGLGVA

SEQ ID NO: 116 - protein, 7 aa, synthetic construct
GFSLNDY

SEQ ID NO: 117 - protein, 5 aa, synthetic construct
DVGGS

SEQ ID NO: 118 - protein, 9 aa, synthetic construct
GGLTYGFDL

SEQ ID NO: 119 - protein, 11 aa, synthetic construct
QASEDISSYLA

SEQ ID NO: 120 - protein, 7 aa, synthetic construct
GASDLAS

SEQ ID NO: 121 - protein, 12 aa, synthetic construct
HYYATISGLGVA

SEQ ID NO: 122 - protein, 123 aa, synthetic construct
EVQLVESGGGLVQPGGSLRLSCAASGFSLSTSGVGVGWIRQAPGKGLEWLALIDWDDDKYYSPSLKSRLTISRDNSKNTLYLQLNSLRAEDTAVYYCARGGSDTVLGDWFAYWGQGTLVTVSS

SEQ ID NO: 123 - protein, 106 aa, synthetic construct
DIQLTQSPSSLSASVGDRVTITCRASQSVSPYLAWYQQKPGKAPKLLIYDASSLESGVPSRFSGSGSGTDFTLTISSLQPEDFATYYCQQGYSLWTFGQGTKVEIK

SEQ ID NO: 124 - protein, 7 aa, synthetic construct
TSGVGVG

SEQ ID NO: 125 - protein, 16 aa, synthetic construct
LIDWDDDKYYSPSLKS

SEQ ID NO: 126 - protein, 13 aa, synthetic construct
GGSDTVLGDWFAY

SEQ ID NO: 127 - protein, 11 aa, synthetic construct
RASQSVSPYLA

SEQ ID NO: 128 - protein, 7 aa, synthetic construct
DASSLES

SEQ ID NO: 129 - protein, 8 aa, synthetic construct
QQGYSLWT

SEQ ID NO: 130 - protein, 10 aa, synthetic construct
GFSLSTSGVG

SEQ ID NO: 131 - protein, 7 aa, synthetic construct
IDWDDDK

SEQ ID NO: 132 - protein, 15 aa, synthetic construct
ARGGSDTVLGDWFAY

SEQ ID NO: 133 - protein, 6 aa, synthetic construct
QSVSPY

SEQ ID NO: 134 - protein, 8 aa, synthetic construct
QQGYSLWT

SEQ ID NO: 135 - protein, 9 aa, synthetic construct
GFSLSTSGV

SEQ ID NO: 136 - protein, 5 aa, synthetic construct
DWDDD

SEQ ID NO: 137 - protein, 13 aa, synthetic construct
GGSDTVLGDWFAY

SEQ ID NO: 138 - protein, 11 aa, synthetic construct
RASQSVSPYLA

SEQ ID NO: 139 - protein, 7 aa, synthetic construct
DASSLES

SEQ ID NO: 140 - protein, 8 aa, synthetic construct
QQGYSLWT

SEQ ID NO: 141 - protein, 123 aa, synthetic construct
EVQLVESGGGLVQPGGSLRLSCAASGFSLSTGGVGVGWIRQAPGKGLEWLALIDWADDKYYSPSLKSRLTISRDNSKNTLYLQLNSLRAEDTAVYYCARGGSDTVIGDWFAYWGQGTLVTVSS

SEQ ID NO: 142 - protein, 106 aa, synthetic construct
DIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIK

SEQ ID NO: 143 - protein, 7 aa, synthetic construct
TGGVGVG

SEQ ID NO: 144 - protein, 16 aa, synthetic construct
LIDWADDKYYSPSLKS

SEQ ID NO: 145 - protein, 13 aa, synthetic construct
GGSDTVIGDWFAY

SEQ ID NO: 146 - protein, 11 aa, synthetic construct
RASQSIGSYLA

SEQ ID NO: 147 - protein, 7 aa, synthetic construct
DASNLET

SEQ ID NO: 148 - protein, 8 aa, synthetic construct
QQGYYLWT

SEQ ID NO: 149 - protein, 10 aa, synthetic construct
GFSLSTGGVG

SEQ ID NO: 150 - protein, 7 aa, synthetic construct
IDWADDK

SEQ ID NO: 151 - protein, 15 aa, synthetic construct
ARGGSDTVIGDWFAY

SEQ ID NO: 152 - protein, 6 aa, synthetic construct
QSIGSY

SEQ ID NO: 153 - protein, 8 aa, synthetic construct
QQGYYLWT

SEQ ID NO: 154 - protein, 9 aa, synthetic construct
GFSLSTGGV

SEQ ID NO: 155 - protein, 5 aa, synthetic construct
DWADD

SEQ ID NO: 156 - protein, 13 aa, synthetic construct
GGSDTVIGDWFAY

SEQ ID NO: 157 - protein, 11 aa, synthetic construct
RASQSIGSYLA

SEQ ID NO: 158 - protein, 7 aa, synthetic construct
DASNLET

SEQ ID NO: 159 - protein, 8 aa, synthetic construct
QQGYYLWT

SEQ ID NO: 160 - protein, 120 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAREPGYYGSGLDYWGQGTLVTVSS

SEQ ID NO: 161 - protein, 109 aa, synthetic construct
SYVLTQPPSVSVAPGETARITCGGDDIGDKRVHWYQKKPDQAPVLVVYEDRYRPSGIPERISGSNSGNTATLTLSRVEAGDEADYYCQVWDSSSDHPGVFGGGTQLIIL

SEQ ID NO: 162 - protein, 5 aa, synthetic construct
GYYMH

SEQ ID NO: 163 - protein, 17 aa, synthetic construct
WINPNSGGTNYAQKFQG

SEQ ID NO: 164 - protein, 11 aa, synthetic construct
EPGYYGSGLDY

SEQ ID NO: 165 - protein, 11 aa, synthetic construct
GGDDIGDKRVH

SEQ ID NO: 166 - protein, 7 aa, synthetic construct
EDRYRPS

SEQ ID NO: 167 - protein, 12 aa, synthetic construct
QVWDSSSDHPGV

SEQ ID NO: 168 - protein, 8 aa, synthetic construct
GYTFTGYY

SEQ ID NO: 169 - protein, 8 aa, synthetic construct
INPNSGGT

SEQ ID NO: 170 - protein, 13 aa, synthetic construct
AREPGYYGSGLDY

SEQ ID NO: 171 - protein, 6 aa, synthetic construct
DIGDKR

SEQ ID NO: 172 - protein, 12 aa, synthetic construct
QVWDSSSDHPGV

SEQ ID NO: 173 - protein, 7 aa, synthetic construct
GYTFTGY

SEQ ID NO: 174 - protein, 6 aa, synthetic construct
NPNSGG

SEQ ID NO: 175 - protein, 11 aa, synthetic construct
EPGYYGSGLDY

SEQ ID NO: 176 - protein, 11 aa, synthetic construct
GGDDIGDKRVH

SEQ ID NO: 177 - protein, 7 aa, synthetic construct
EDRYRPS

SEQ ID NO: 178 - protein, 12 aa, synthetic construct
QVWDSSSDHPGV

SEQ ID NO: 179 - protein, 118 aa, synthetic construct
EVQLLESGGGLVQPGGSLRLSCAASGFTFGYSYMSWVRQAPGKGLEWVSSIGSGSSYTYYADSVKGRFTISRDNSKNTLYLQMNSLRAEDTAVYYCARVYSSPGIDYWGQGTLVTVSS

SEQ ID NO: 180 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQYYTWVPFTFGQGTKLEIK

SEQ ID NO: 181 - protein, 5 aa, synthetic construct
YSYMS

SEQ ID NO: 182 - protein, 17 aa, synthetic construct
SIGSGSSYTYYADSVKG

SEQ ID NO: 183 - protein, 9 aa, synthetic construct
VYSSPGIDY

SEQ ID NO: 184 - protein, 11 aa, synthetic construct
RASQSISSYLN

SEQ ID NO: 185 - protein, 7 aa, synthetic construct
AASSLQS

SEQ ID NO: 186 - protein, 10 aa, synthetic construct
QQYYTWVPFT

SEQ ID NO: 187 - protein, 8 aa, synthetic construct
GFTFGYSY

SEQ ID NO: 188 - protein, 8 aa, synthetic construct
IGSGSSYT

SEQ ID NO: 189 - protein, 11 aa, synthetic construct
ARVYSSPGIDY

SEQ ID NO: 190 - protein, 6 aa, synthetic construct
QSISSY

SEQ ID NO: 191 - protein, 10 aa, synthetic construct
QQYYTWVPFT

SEQ ID NO: 192 - protein, 7 aa, synthetic construct
GFTFGYS

SEQ ID NO: 193 - protein, 6 aa, synthetic construct
GSGSSY

SEQ ID NO: 194 - protein, 9 aa, synthetic construct
VYSSPGIDY

SEQ ID NO: 195 - protein, 11 aa, synthetic construct
RASQSISSYLN

SEQ ID NO: 196 - protein, 7 aa, synthetic construct
AASSLQS

SEQ ID NO: 197 - protein, 10 aa, synthetic construct
QQYYTWVPFT

SEQ ID NO: 198 - protein, 118 aa, synthetic construct
EVQLLESGGGLVQPGGSLRLSCAASGFNFGYSYMSWVRQAPGKGLEWVSSIGSTSSHTYYADSVKGRFTISRDNSKNTLYLQMNSLRAEDTAVYYCARVYSSPGIDYWGQGTLVTVSS

SEQ ID NO: 199 - protein, 108 aa, synthetic construct
DIQMTQSPSSLSASVGDRVTITCRASQSIGSTLNWYQQKPGKAPKLLIYGASSLQSGVPSRFSGSGSGTDFTLTISSLQPEDFATYYCQQYYTWVPFTFGQGTKLEIK

SEQ ID NO: 200 - protein, 5 aa, synthetic construct
YSYMS

SEQ ID NO: 201 - protein, 17 aa, synthetic construct
SIGSTSSHTYYADSVKG

SEQ ID NO: 202 - protein, 9 aa, synthetic construct
VYSSPGIDY

SEQ ID NO: 203 - protein, 11 aa, synthetic construct
RASQSIGSTLN

SEQ ID NO: 204 - protein, 7 aa, synthetic construct
GASSLQS

SEQ ID NO: 205 - protein, 10 aa, synthetic construct
QQYYTWVPFT

SEQ ID NO: 206 - protein, 8 aa, synthetic construct
GFNFGYSY

SEQ ID NO: 207 - protein, 8 aa, synthetic construct
IGSTSSHT

SEQ ID NO: 208 - protein, 11 aa, synthetic construct
ARVYSSPGIDY

SEQ ID NO: 209 - protein, 6 aa, synthetic construct
QSIGST

SEQ ID NO: 210 - protein, 10 aa, synthetic construct
QQYYTWVPFT

SEQ ID NO: 211 - protein, 7 aa, synthetic construct
GFNFGYS

SEQ ID NO: 212 - protein, 6 aa, synthetic construct
GSTSSH

SEQ ID NO: 213 - protein, 9 aa, synthetic construct
VYSSPGIDY

SEQ ID NO: 214 - protein, 11 aa, synthetic construct
RASQSIGSTLN

SEQ ID NO: 215 - protein, 7 aa, synthetic construct
GASSLQS

SEQ ID NO: 216 - protein, 10 aa, synthetic construct
QQYYTWVPFT

SEQ ID NO: 217 - protein, 126 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDSPFLLDDYYYYYYMDVWGKGTTVTVSS

SEQ ID NO: 218 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQGHLFPITFGGGTKVEIK

SEQ ID NO: 219 - protein, 5 aa, synthetic construct
SYAMS

SEQ ID NO: 220 - protein, 17 aa, synthetic construct
AISGSGGSTYYADSVKG

SEQ ID NO: 221 - protein, 17 aa, synthetic construct
DSPFLLDDYYYYYYMDV

SEQ ID NO: 222 - protein, 11 aa, synthetic construct
RASQGISSWLA

SEQ ID NO: 223 - protein, 7 aa, synthetic construct
AASSLQS

SEQ ID NO: 224 - protein, 9 aa, synthetic construct
QQGHLFPIT

SEQ ID NO: 225 - protein, 8 aa, synthetic construct
GFTFSSYA

SEQ ID NO: 226 - protein, 8 aa, synthetic construct
ISGSGGST

SEQ ID NO: 227 - protein, 19 aa, synthetic construct
AKDSPFLLDDYYYYYYMDV

SEQ ID NO: 228 - protein, 6 aa, synthetic construct
QGISSW

SEQ ID NO: 229 - protein, 9 aa, synthetic construct
QQGHLFPIT

SEQ ID NO: 230 - protein, 7 aa, synthetic construct
GFTFSSY

SEQ ID NO: 231 - protein, 6 aa, synthetic construct
SGSGGS

SEQ ID NO: 232 - protein, 17 aa, synthetic construct
DSPFLLDDYYYYYYMDV

SEQ ID NO: 233 - protein, 11 aa, synthetic construct
RASQGISSWLA

SEQ ID NO: 234 - protein, 7 aa, synthetic construct
AASSLQS

SEQ ID NO: 235 - protein, 9 aa, synthetic construct
QQGHLFPIT

SEQ ID NO: 236 - protein, 126 aa, synthetic construct
EVQLLESGGGLVQPGGSLRLSCAASGFTFYGYAMSWVRQAPGKGLEWVAAISGSGDSTYYADSVKGRFTISRDNSKNTLYLQMNSLRAEDTAVYYCAKDSPFLLDDYYYYYYMDVWGKGTTVTVSS

SEQ ID NO: 237 - protein, 107 aa, synthetic construct; identical to SEQ ID NO: 218

SEQ ID NO: 238 - protein, 5 aa, synthetic construct
GYAMS

SEQ ID NO: 239 - protein, 17 aa, synthetic construct
AISGSGDSTYYADSVKG

SEQ ID NO: 240 - protein, 17 aa, synthetic construct
DSPFLLDDYYYYYYMDV

SEQ ID NO: 241 - protein, 11 aa, synthetic construct
RASQGISSWLA

SEQ ID NO: 242 - protein, 7 aa, synthetic construct
AASSLQS

SEQ ID NO: 243 - protein, 9 aa, synthetic construct
QQGHLFPIT

SEQ ID NO: 244 - protein, 8 aa, synthetic construct
GFTFYGYA

SEQ ID NO: 245 - protein, 8 aa, synthetic construct
ISGSGDST

SEQ ID NO: 246 - protein, 19 aa, synthetic construct
AKDSPFLLDDYYYYYYMDV

SEQ ID NO: 247 - protein, 6 aa, synthetic construct
QGISSW

SEQ ID NO: 248 - protein, 9 aa, synthetic construct
QQGHLFPIT

SEQ ID NO: 249 - protein, 7 aa, synthetic construct
GFTFYGY

SEQ ID NO: 250 - protein, 6 aa, synthetic construct
SGSGDS

SEQ ID NO: 251 - protein, 17 aa, synthetic construct
DSPFLLDDYYYYYYMDV

SEQ ID NO: 252 - protein, 11 aa, synthetic construct
RASQGISSWLA

SEQ ID NO: 253 - protein, 7 aa, synthetic construct
AASSLQS

SEQ ID NO: 254 - protein, 9 aa, synthetic construct
QQGHLFPIT

SEQ ID NO: 255 - protein, 126 aa, synthetic construct
EVQLLESGGGLVQPGGSLRLSCAASGFTFRNYAMSWVRQAPGKGLEWVSAISGSGDTTYYADSVKGRFTISRDNSKNTLYLQMNSLRAEDTAVYYCAKDSPFLLDDYYYYYYMDVWGKGTTVTVSS

SEQ ID NO: 256 - protein, 107 aa, synthetic construct; identical to SEQ ID NO: 218

SEQ ID NO: 257 - protein, 5 aa, synthetic construct
NYAMS

SEQ ID NO: 258 - protein, 17 aa, synthetic construct
AISGSGDTTYYADSVKG

SEQ ID NO: 259 - protein, 17 aa, synthetic construct
DSPFLLDDYYYYYYMDV

SEQ ID NO: 260 - protein, 11 aa, synthetic construct
RASQGISSWLA

SEQ ID NO: 261 - protein, 7 aa, synthetic construct
AASSLQS

SEQ ID NO: 262 - protein, 9 aa, synthetic construct
QQGHLFPIT

SEQ ID NO: 263 - protein, 8 aa, synthetic construct
GFTFRNYA

SEQ ID NO: 264 - protein, 8 aa, synthetic construct
ISGSGDTT

SEQ ID NO: 265 - protein, 19 aa, synthetic construct
AKDSPFLLDDYYYYYYMDV

SEQ ID NO: 266 - protein, 6 aa, synthetic construct
QGISSW

SEQ ID NO: 267 - protein, 9 aa, synthetic construct
QQGHLFPIT

SEQ ID NO: 268 - protein, 7 aa, synthetic construct
GFTFRNY

SEQ ID NO: 269 - protein, 6 aa, synthetic construct
SGSGDT

SEQ ID NO: 270 - protein, 17 aa, synthetic construct
DSPFLLDDYYYYYYMDV

SEQ ID NO: 271 - protein, 11 aa, synthetic construct
RASQGISSWLA

SEQ ID NO: 272 - protein, 7 aa, synthetic construct
AASSLQS

SEQ ID NO: 273 - protein, 9 aa, synthetic construct
QQGHLFPIT

SEQ ID NO: 274 - protein, 116 aa, synthetic construct
QVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSS

SEQ ID NO: 275 - protein, 96 aa, synthetic construct
DIQMTQSPSSLSASVGDRVTITCRASQDIRSNLNWYQQKPGGAVKLLIYYTSRLHSGVPSRFSGSGSGTDYTLTISSLQPEDFATYFCQQSEKLPR

SEQ ID NO: 276 - protein, 5 aa, synthetic construct
GFEMH

SEQ ID NO: 277 - protein, 17 aa, synthetic construct
AIDPKTGGTDYNQKFKD

SEQ ID NO: 278 - protein, 7 aa, synthetic construct
DLGYFDV

SEQ ID NO: 279 - protein, 11 aa, synthetic construct
RASQDIRSNLN

SEQ ID NO: 280 - protein, 7 aa, synthetic construct
YTSRLHS

SEQ ID NO: 281 - protein, 8 aa, synthetic construct
QQSEKLPR

SEQ ID NO: 282 - protein, 8 aa, synthetic construct
GYTFAGFE

SEQ ID NO: 283 - protein, 8 aa, synthetic construct
IDPKTGGT

SEQ ID NO: 284 - protein, 9 aa, synthetic construct
ARDLGYFDV

SEQ ID NO: 285 - protein, 6 aa, synthetic construct
QDIRSN

SEQ ID NO: 286 - protein, 8 aa, synthetic construct
QQSEKLPR

SEQ ID NO: 287 - protein, 7 aa, synthetic construct
GYTFAGF

SEQ ID NO: 288 - protein, 6 aa, synthetic construct
DPKTGG

SEQ ID NO: 289 - protein, 7 aa, synthetic construct
DLGYFDV

SEQ ID NO: 290 - protein, 11 aa, synthetic construct
RASQDIRSNLN

SEQ ID NO: 291 - protein, 7 aa, synthetic construct
YTSRLHS

SEQ ID NO: 292 - protein, 8 aa, synthetic construct
QQSEKLPR

SEQ ID NO: 293 - protein, 121 aa, synthetic construct [sequence omitted]

SEQ ID NO: 294 - protein, 109 aa, synthetic construct [sequence omitted]

SEQ ID NO: 295 - protein, 5 aa, synthetic construct
GYYWS

SEQ ID NO: 296 - protein, 16 aa, synthetic construct
EINHGGYVTYNPSLES

SEQ ID NO: 297 - protein, 13 aa, synthetic construct
DYGPGNYDWYFDL

SEQ ID NO: 298 - protein, 11 aa, synthetic construct
RASQSVSSYLA

SEQ ID NO: 299 - protein, 7 aa, synthetic construct
DASNRAT

SEQ ID NO: 300 - protein, 11 aa, synthetic construct
QQRSNWPPALT

SEQ ID NO: 301 - protein, 8 aa, synthetic construct
GGSFSGYY

SEQ ID NO: 302 - protein, 7 aa, synthetic construct
INHGGYV

SEQ ID NO: 303 - protein, 15 aa, synthetic construct
ARDYGPGNYDWYFDL

SEQ ID NO: 304 - protein, 6 aa, synthetic construct
QSVSSY

SEQ ID NO: 305 - protein, 11 aa, synthetic construct
QQRSNWPPALT

SEQ ID NO: 306 - protein, 7 aa, synthetic construct
GGSFSGY

SEQ ID NO: 307 - protein, 5 aa, synthetic construct
NHGGY

SEQ ID NO: 308 - protein, 13 aa, synthetic construct
DYGPGNYDWYFDL

SEQ ID NO: 309 - protein, 11 aa, synthetic construct
RASQSVSSYLA

SEQ ID NO: 310 - protein, 7 aa, synthetic construct
DASNRAT

SEQ ID NO: 311 - protein, 11 aa, synthetic construct
QQRSNWPPALT

SEQ ID NO: 312 - protein, 116 aa, synthetic construct
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSS

SEQ ID NO: 313 - protein, 108 aa, synthetic construct
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVL

SEQ ID NO: 314 - protein, 5 aa, synthetic construct
TYWIS

SEQ ID NO: 315 - protein, 17 aa, synthetic construct
KIYPGDSYTNYSPSFQG

SEQ ID NO: 316 - protein, 7 aa, synthetic construct
GYGIFDY

SEQ ID NO: 317 - protein, 11 aa, synthetic construct
SGDNIGDQYAH

SEQ ID NO: 318 - protein, 7 aa, synthetic construct
QDKNRPS

SEQ ID NO: 319 - protein, 11 aa, synthetic construct
ATYTGFGSLAV

SEQ ID NO: 320 - protein, 8 aa, synthetic construct
GYSFSTYW

SEQ ID NO: 321 - protein, 8 aa, synthetic construct
IYPGDSYT

SEQ ID NO: 322 - protein, 9 aa, synthetic construct
ARGYGIFDY

SEQ ID NO: 323 - protein, 6 aa, synthetic construct
NIGDQY

SEQ ID NO: 324 - protein, 11 aa, synthetic construct
ATYTGFGSLAV

SEQ ID NO: 325 - protein, 7 aa, synthetic construct
GYSFSTY

SEQ ID NO: 326 - protein, 6 aa, synthetic construct
YPGDSY

SEQ ID NO: 327 - protein, 7 aa, synthetic construct
GYGIFDY

SEQ ID NO: 328 - protein, 11 aa, synthetic construct
SGDNIGDQYAH

SEQ ID NO: 329 - protein, 7 aa, synthetic construct
QDKNRPS

SEQ ID NO: 330 - protein, 11 aa, synthetic construct
ATYTGFGSLAV

SEQ ID NO: 331 - protein, 118 aa, synthetic construct
EVQLVESGGGLVQPGGSLRLSCAASGFTFSSYSMNWVRQAPGKGLEWVSYISSSSSTIDYADSVKGRFTISRDNAKNSLYLQMNSLRDEDTAVYYCARESGWYLFDYWGQGTLVTVSS

SEQ ID NO: 332 - protein, 107 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGGGTKVEIK

SEQ ID NO: 333 - protein, 5 aa, synthetic construct
SYSMN

SEQ ID NO: 334 - protein, 17 aa, synthetic construct
YISSSSSTIDYADSVKG

SEQ ID NO: 335 - protein, 9 aa, synthetic construct
ESGWYLFDY

SEQ ID NO: 336 - protein, 11 aa, synthetic construct
RASQGISSWLA

SEQ ID NO: 337 - protein, 7 aa, synthetic construct
AASSLQS

SEQ ID NO: 338 - protein, 9 aa, synthetic construct
QQYNSYPPT

SEQ ID NO: 339 - protein, 8 aa, synthetic construct
GFTFSSYS

SEQ ID NO: 340 - protein, 8 aa, synthetic construct
ISSSSSTI

SEQ ID NO: 341 - protein, 11 aa, synthetic construct
ARESGWYLFDY

SEQ ID NO: 342 - protein, 6 aa, synthetic construct
QGISSW

SEQ ID NO: 343 - protein, 9 aa, synthetic construct
QQYNSYPPT

SEQ ID NO: 344 - protein, 7 aa, synthetic construct
GFTFSSY

SEQ ID NO: 345 - protein, 6 aa, synthetic construct
SSSSST

SEQ ID NO: 346 - protein, 9 aa, synthetic construct
ESGWYLFDY

SEQ ID NO: 347 - protein, 11 aa, synthetic construct
RASQGISSWLA

SEQ ID NO: 348 - protein, 7 aa, synthetic construct
AASSLQS

SEQ ID NO: 349 - protein, 9 aa, synthetic construct
QQYNSYPPT

SEQ ID NO: 350 - protein, 124 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARGRPWYSETGTSAFDIWGQGTMVTVSS

SEQ ID NO: 351 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQSDHYPTFGGGTKVEIKR

SEQ ID NO: 352 - protein, 5 aa, synthetic construct
SYGMH

SEQ ID NO: 353 - protein, 17 aa, synthetic construct
VISYDGSNKYYADSVKG

SEQ ID NO: 354 - protein, 15 aa, synthetic construct
GRPWYSETGTSAFDI

SEQ ID NO: 355 - protein, 11 aa, synthetic construct
QASQDISNYLN

SEQ ID NO: 356 - protein, 7 aa, synthetic construct
DASNLET

SEQ ID NO: 357 - protein, 8 aa, synthetic construct
QQSDHYPT

SEQ ID NO: 358 - protein, 8 aa, synthetic construct
GFTFSSYG

SEQ ID NO: 359 - protein, 8 aa, synthetic construct
ISYDGSNK

SEQ ID NO: 360 - protein, 17 aa, synthetic construct
ARGRPWYSETGTSAFDI

SEQ ID NO: 361 - protein, 6 aa, synthetic construct
QDISNY

SEQ ID NO: 362 - protein, 8 aa, synthetic construct
QQSDHYPT

SEQ ID NO: 363 - protein, 7 aa, synthetic construct
GFTFSSY

SEQ ID NO: 364 - protein, 6 aa, synthetic construct
SYDGSN

SEQ ID NO: 365 - protein, 15 aa, synthetic construct
GRPWYSETGTSAFDI

SEQ ID NO: 366 - protein, 11 aa, synthetic construct
QASQDISNYLN

SEQ ID NO: 367 - protein, 7 aa, synthetic construct
DASNLET

SEQ ID NO: 368 - protein, 8 aa, synthetic construct
QQSDHYPT

SEQ ID NO: 369 - protein, 122 aa, synthetic construct
QVQLVQSGSELKKPGASVKVSCKASGYTFTDYSMHWVRQAPGQGLKWMGWINTETGEPTYADDFKGRFVFSLDTSVSTAYLQISSLKAEDTAVYYCANPYYDYVSYYAMDYWGQGTLVTVSS

SEQ ID NO: 370 - protein, 107 aa, synthetic construct
DIQMTQSPSSLSASVGDRVTITCKASQDVSTAVAWYQQKPGKAPKLLIYSASYLYTGVPSRFSGSGSGTDFTFTISSLQPEDIATYYCQQHYSTPRTFGQGTKLEIK

SEQ ID NO: 371 - protein, 5 aa, synthetic construct
DYSMH

SEQ ID NO: 372 - protein, 17 aa, synthetic construct
WINTETGEPTYADDFKG

SEQ ID NO: 373 - protein, 13 aa, synthetic construct
PYYDYVSYYAMDY

SEQ ID NO: 374 - protein, 11 aa, synthetic construct
KASQDVSTAVA

SEQ ID NO: 375 - protein, 7 aa, synthetic construct
SASYLYT

SEQ ID NO: 376 - protein, 9 aa, synthetic construct
QQHYSTPRT

SEQ ID NO: 377 - protein, 8 aa, synthetic construct
GYTFTDYS

SEQ ID NO: 378 - protein, 8 aa, synthetic construct
INTETGEP

SEQ ID NO: 379 - protein, 15 aa, synthetic construct
ANPYYDYVSYYAMDY

SEQ ID NO: 380 - protein, 6 aa, synthetic construct
QDVSTA

SEQ ID NO: 381 - protein, 9 aa, synthetic construct
QQHYSTPRT

SEQ ID NO: 382 - protein, 7 aa, synthetic construct
GYTFTDY

SEQ ID NO: 383 - protein, 6 aa, synthetic construct
NTETGE

SEQ ID NO: 384 - protein, 13 aa, synthetic construct
PYYDYVSYYAMDY

SEQ ID NO: 385 - protein, 11 aa, synthetic construct
KASQDVSTAVA

SEQ ID NO: 386 - protein, 7 aa, synthetic construct
SASYLYT

SEQ ID NO: 387 - protein, 9 aa, synthetic construct
QQHYSTPRT

SEQ ID NO: 388 - protein, 120 aa, synthetic construct
EVQLVESGGGLVQPGGSLRLSCAASEYEFPSHDMSWVRQAPGKGLELVAAINSDGGSTYYPDTMERRFTISRDNAKNSLYLQMNSLRAEDTAVYYCARHYDDYYAWFAYWGQGTLVTVSS

SEQ ID NO: 389 - protein, 111 aa, synthetic construct
EIVLTQSPATLSLSPGERATLSCRASKSVSTSGYSYMHWYQQKPGQAPRLLIYLASNLESGVPARFSGSGSGTDFTLTISSLEPEDFAVYYCQHSRELPLTFGGGTKVEIK

SEQ ID NO: 390 - protein, 5 aa, synthetic construct
SHDMS

SEQ ID NO: 391 - protein, 17 aa, synthetic construct
AINSDGGSTYYPDTMER

SEQ ID NO: 392 - protein, 11 aa, synthetic construct
HYDDYYAWFAY

SEQ ID NO: 393 - protein, 15 aa, synthetic construct
RASKSVSTSGYSYMH

SEQ ID NO: 394 - protein, 7 aa, synthetic construct
LASNLES

SEQ ID NO: 395 - protein, 9 aa, synthetic construct
QHSRELPLT

SEQ ID NO: 396 - protein, 8 aa, synthetic construct
EYEFPSHD

SEQ ID NO: 397 - protein, 8 aa, synthetic construct
INSDGGST

SEQ ID NO: 398 - protein, 13 aa, synthetic construct
ARHYDDYYAWFAY

SEQ ID NO: 399 - protein, 10 aa, synthetic construct
KSVSTSGYSY

SEQ ID NO: 400 - protein, 9 aa, synthetic construct
QHSRELPLT

SEQ ID NO: 401 - protein, 7 aa, synthetic construct
EYEFPSH

SEQ ID NO: 402 - protein, 6 aa, synthetic construct
NSDGGS

SEQ ID NO: 403 - protein, 11 aa, synthetic construct
HYDDYYAWFAY

SEQ ID NO: 404 - protein, 15 aa, synthetic construct
RASKSVSTSGYSYMH

SEQ ID NO: 405 - protein, 7 aa, synthetic construct
LASNLES

SEQ ID NO: 406 - protein, 9 aa, synthetic construct
QHSRELPLT

SEQ ID NO: 407 - protein, 121 aa, synthetic construct
EVQLVESGGGLVQPGGSLKLSCAASGFTFSGSAMHWVRQASGKGLEWVGRIRSKANSYATAYAASVKGRFTISRDDSKNTAYLQMNSLKTEDTAVYYCTSGIYDSSGYDYWGQGTLVTVSS

SEQ ID NO: 408 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 409 - protein, 5 aa, synthetic construct
GSAMH

SEQ ID NO: 410 - protein, 19 aa, synthetic construct
RIRSKANSYATAYAASVKG

SEQ ID NO: 411 - protein, 10 aa, synthetic construct
GIYDSSGYDY

SEQ ID NO: 412 - protein, 16 aa, synthetic construct
RSSQSLLHSNGYNYLD

SEQ ID NO: 413 - protein, 7 aa, synthetic construct
LGSNRAS

SEQ ID NO: 414 - protein, 9 aa, synthetic construct
MQALQTPLT

SEQ ID NO: 415 - protein, 8 aa, synthetic construct
GFTFSGSA

SEQ ID NO: 416 - protein, 10 aa, synthetic construct
IRSKANSYAT

SEQ ID NO: 417 - protein, 12 aa, synthetic construct
TSGIYDSSGYDY

SEQ ID NO: 418 - protein, 11 aa, synthetic construct
QSLLHSNGYNY

SEQ ID NO: 419 - protein, 9 aa, synthetic construct
MQALQTPLT

SEQ ID NO: 420 - protein, 7 aa, synthetic construct
GFTFSGS

SEQ ID NO: 421 - protein, 8 aa, synthetic construct
RSKANSYA

SEQ ID NO: 422 - protein, 10 aa, synthetic construct
GIYDSSGYDY

SEQ ID NO: 423 - protein, 16 aa, synthetic construct
RSSQSLLHSNGYNYLD

SEQ ID NO: 424 - protein, 7 aa, synthetic construct
LGSNRAS

SEQ ID NO: 425 - protein, 9 aa, synthetic construct
MQALQTPLT

SEQ ID NO: 426 - protein, 118 aa, synthetic construct; identical to SEQ ID NO: 331

SEQ ID NO: 427 - protein, 108 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGGGTKVEIKR

SEQ ID NO: 428 - protein, 5 aa, synthetic construct
SYSMN

SEQ ID NO: 429 - protein, 17 aa, synthetic construct
YISSSSSTIDYADSVKG

SEQ ID NO: 430 - protein, 9 aa, synthetic construct
ESGWYLFDY

SEQ ID NO: 431 - protein, 11 aa, synthetic construct
RASQGISSWLA

SEQ ID NO: 432 - protein, 7 aa, synthetic construct
AASSLQS

SEQ ID NO: 433 - protein, 9 aa, synthetic construct
QQYNSYPPT

SEQ ID NO: 434 - protein, 8 aa, synthetic construct
GFTFSSYS

SEQ ID NO: 435 - protein, 8 aa, synthetic construct
ISSSSSTI

SEQ ID NO: 436 - protein, 11 aa, synthetic construct
ARESGWYLFDY

SEQ ID NO: 437 - protein, 6 aa, unidentified
QGISSW

SEQ ID NO: 438 - protein, 9 aa, synthetic construct
QQYNSYPPT

SEQ ID NO: 439 - protein, 7 aa, synthetic construct
GFTFSSY

SEQ ID NO: 440 - protein, 6 aa, synthetic construct
SSSSST

SEQ ID NO: 441 - protein, 9 aa, synthetic construct
ESGWYLFDY

SEQ ID NO: 442 - protein, 11 aa, synthetic construct
RASQGISSWLA

SEQ ID NO: 443 - protein, 7 aa, synthetic construct
AASSLQS

SEQ ID NO: 444 - protein, 9 aa, synthetic construct
QQYNSYPPT

SEQ ID NO: 445 - protein, 121 aa, synthetic construct
EVQLQESGPSLVKPSQTLSLTCSVTGDSFTSGYWNWIRKFPGNRLEYMGYISYNGITYHNPSLKSRISITRDTSKNHYYLQLNSVTTEDTATYFCARYRYDYDGGHAMDYWGQGTLVTVSS

SEQ ID NO: 446 - protein, 107 aa, synthetic construct
DIQMTQTTSSLSASLGDRVTISCRASQDISNYLNWYQQKPDGTVKLLIYYTSKLHSGVPSRFSGSGSRTDYSLTITDLDQEDIATYFCQQGSALPWTFGQGTKVEIK

SEQ ID NO: 447 - protein, 5 aa, synthetic construct
SGYWN

SEQ ID NO: 448 - protein, 16 aa, synthetic construct
YISYNGITYHNPSLKS

SEQ ID NO: 449 - protein, 13 aa, synthetic construct
YRYDYDGGHAMDY

SEQ ID NO: 450 - protein, 11 aa, synthetic construct
RASQDISNYLN

SEQ ID NO: 451 - protein, 7 aa, synthetic construct
YTSKLHS

SEQ ID NO: 452 - protein, 9 aa, synthetic construct
QQGSALPWT

SEQ ID NO: 453 - protein, 8 aa, synthetic construct
GDSFTSGY

SEQ ID NO: 454 - protein, 7 aa, synthetic construct
ISYNGIT

SEQ ID NO: 455 - protein, 15 aa, synthetic construct
ARYRYDYDGGHAMDY

SEQ ID NO: 456 - protein, 6 aa, synthetic construct
QDISNY

SEQ ID NO: 457 - protein, 9 aa, synthetic construct
QQGSALPWT

SEQ ID NO: 458 - protein, 7 aa, synthetic construct
GDSFTSG

SEQ ID NO: 459 - protein, 5 aa, synthetic construct
SYNGI

SEQ ID NO: 460 - protein, 13 aa, synthetic construct
YRYDYDGGHAMDY

SEQ ID NO: 461 - protein, 11 aa, synthetic construct
RASQDISNYLN

SEQ ID NO: 462 - protein, 7 aa, synthetic construct
YTSKLHS

SEQ ID NO: 463 - protein, 9 aa, synthetic construct
QQGSALPWT

SEQ ID NO: 464 - protein, 117 aa, synthetic construct
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSS

SEQ ID NO: 465 - protein, 107 aa, synthetic construct
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIK

SEQ ID NO: 466 - protein, 5 aa, synthetic construct
DSYMS

SEQ ID NO: 467 - protein, 17 aa, synthetic construct
DMYPDNGDSSYNQKFRE

SEQ ID NO: 468 - protein, 8 aa, synthetic construct
APRWYFSV

SEQ ID NO: 469 - protein, 11 aa, synthetic construct
RASQDISNYLN

SEQ ID NO: 470 - protein, 7 aa, synthetic construct
YTSRLRS

SEQ ID NO: 471 - protein, 9 aa, synthetic construct
QQGHTLPPT

SEQ ID NO: 472 - protein, 8 aa, synthetic construct
GYTFTDSY

SEQ ID NO: 473 - protein, 8 aa, synthetic construct
MYPDNGDS

SEQ ID NO: 474 - protein, 10 aa, synthetic construct
VLAPRWYFSV

SEQ ID NO: 475 - protein, 6 aa, synthetic construct
QDISNY

SEQ ID NO: 476 - protein, 9 aa, synthetic construct
QQGHTLPPT

SEQ ID NO: 477 - protein, 7 aa, synthetic construct
GYTFTDS

SEQ ID NO: 478 - protein, 6 aa, synthetic construct
YPDNGD

SEQ ID NO: 479 - protein, 8 aa, synthetic construct
APRWYFSV

SEQ ID NO: 480 - protein, 11 aa, synthetic construct
RASQDISNYLN

SEQ ID NO: 481 - protein, 7 aa, synthetic construct
YTSRLRS

SEQ ID NO: 482 - protein, 9 aa, synthetic construct
QQGHTLPPT

SEQ ID NO: 483 - protein, 120 aa, synthetic construct
EVQLVESGGGLVQPGGSLRLSCAASGFTFSRYGMSWVRQAPGKGLELVATINSNGGRTYYPDSVKGRFTISRDNAKNSLYLQMNSLRAEDTAVYYCAREGITTAYAMDYWGQGTTVTVSS

SEQ ID NO: 484 - protein, 112 aa, synthetic construct
DIVMTQSPDSLAVSLGERATINCKASQSVDYDGDSYMHWYQQKPGQPPKLLIYAASILESGVPDRFSGSGSGTDFTLTISSLQAEDVAVYYCQQSNEDPRTFGGGTKVEIKR

SEQ ID NO: 485 - protein, 5 aa, synthetic construct
RYGMS

SEQ ID NO: 486 - protein, 17 aa, synthetic construct
TINSNGGRTYYPDSVKG

SEQ ID NO: 487 - protein, 11 aa, synthetic construct
EGITTAYAMDY

SEQ ID NO: 488 - protein, 15 aa, synthetic construct
KASQSVDYDGDSYMH

SEQ ID NO: 489 - protein, 7 aa, synthetic construct
AASILES

SEQ ID NO: 490 - protein, 9 aa, synthetic construct
QQSNEDPRT

SEQ ID NO: 491 - protein, 8 aa, synthetic construct
GFTFSRYG

SEQ ID NO: 492 - protein, 8 aa, synthetic construct
INSNGGRT

SEQ ID NO: 493 - protein, 13 aa, synthetic construct
AREGITTAYAMDY

SEQ ID NO: 494 - protein, 10 aa, synthetic construct
QSVDYDGDSY

SEQ ID NO: 495 - protein, 9 aa, synthetic construct
QQSNEDPRT

SEQ ID NO: 496 - protein, 7 aa, synthetic construct
GFTFSRY

SEQ ID NO: 497 - protein, 6 aa, synthetic construct
NSNGGR

SEQ ID NO: 498 - protein, 11 aa, synthetic construct
EGITTAYAMDY

SEQ ID NO: 499 - protein, 15 aa, synthetic construct
KASQSVDYDGDSYMH

SEQ ID NO: 500 - protein, 7 aa, synthetic construct
AASILES

SEQ ID NO: 501 - protein, 9 aa, synthetic construct
QQSNEDPRT

SEQ ID NO: 502 - protein, 121 aa, synthetic construct
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSS

SEQ ID NO: 503 - protein, 108 aa, synthetic construct
DIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIKR

SEQ ID NO: 504 - protein, 5 aa, synthetic construct
SGYWN

SEQ ID NO: 505 - protein, 16 aa, synthetic construct
YISYNGITYHNPSLKS

SEQ ID NO: 506 - protein, 13 aa, synthetic construct
YKYDYDGGHAMDY

SEQ ID NO: 507 - protein, 11 aa, synthetic construct
RASQDISNYLN

SEQ ID NO: 508 - protein, 7 aa, synthetic construct
YTSKLHS

SEQ ID NO: 509 - protein, 9 aa, synthetic construct
QQGSALPWT

SEQ ID NO: 510 - protein, 8 aa, synthetic construct
GGSFSSGY

SEQ ID NO: 511 - protein, 7 aa, synthetic construct
ISYNGIT

SEQ ID NO: 512 - protein, 15 aa, synthetic construct
ARYKYDYDGGHAMDY

SEQ ID NO: 513 - protein, 6 aa, synthetic construct
QDISNY

SEQ ID NO: 514 - protein, 9 aa, synthetic construct
QQGSALPWT

SEQ ID NO: 515 - protein, 7 aa, synthetic construct
GGSFSSG

SEQ ID NO: 516 - protein, 5 aa, synthetic construct
SYNGI

SEQ ID NO: 517 - protein, 13 aa, synthetic construct
YKYDYDGGHAMDY

SEQ ID NO: 518 - protein, 11 aa, synthetic construct
RASQDISNYLN

SEQ ID NO: 519 - protein, 7 aa, synthetic construct
YTSKLHS

SEQ ID NO: 520 - protein, 9 aa, synthetic construct
QQGSALPWT

SEQ ID NO: 521 - protein, 117 aa, synthetic construct; identical to SEQ ID NO: 464

SEQ ID NO: 522 - protein, 108 aa, synthetic construct
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIKR

SEQ ID NO: 523 - protein, 5 aa, synthetic construct
DSYMS

SEQ ID NO: 524 - protein, 17 aa, synthetic construct
DMYPDNGDSSYNQKFRE

SEQ ID NO: 525 - protein, 8 aa, synthetic construct
APRWYFSV

SEQ ID NO: 526 - protein, 11 aa, synthetic construct
RASQDISNYLN

SEQ ID NO: 527 - protein, 7 aa, synthetic construct
YTSRLRS

SEQ ID NO: 528 - protein, 9 aa, synthetic construct
QQGHTLPPT

SEQ ID NO: 529 - protein, 8 aa, synthetic construct
GYTFTDSY

SEQ ID NO: 530 - protein, 8 aa, synthetic construct
MYPDNGDS

SEQ ID NO: 531 - protein, 10 aa, synthetic construct
VLAPRWYFSV

SEQ ID NO: 532 - protein, 6 aa, synthetic construct
QDISNY

SEQ ID NO: 533 - protein, 9 aa, synthetic construct
QQGHTLPPT

SEQ ID NO: 534 - protein, 7 aa, synthetic construct
GYTFTDS

SEQ ID NO: 535 - protein, 6 aa, synthetic construct
YPDNGD

SEQ ID NO: 536 - protein, 8 aa, synthetic construct
APRWYFSV

SEQ ID NO: 537 - protein, 11 aa, synthetic construct
RASQDISNYLN

SEQ ID NO: 538 - protein, 7 aa, synthetic construct
YTSRLRS

SEQ ID NO: 539 - protein, 9 aa, synthetic construct
QQGHTLPPT

SEQ ID NO: 540 - protein, 118 aa, synthetic construct [sequence omitted]

SEQ ID NO: 541 - protein, 108 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 542 - protein, 5 aa, synthetic construct
DSWIH

SEQ ID NO: 543 - protein, 17 aa, synthetic construct
WISPYGGSTYYADSVKG

SEQ ID NO: 544 - protein, 9 aa, synthetic construct
RHWPGGFDY

SEQ ID NO: 545 - protein, 11 aa, synthetic construct
RASQDVSTAVA

SEQ ID NO: 546 - protein, 7 aa, synthetic construct
SASFLYS

SEQ ID NO: 547 - protein, 9 aa, synthetic construct
QQYLYHPAT

SEQ ID NO: 548 - protein, 8 aa, synthetic construct
GFTFSDSW

SEQ ID NO: 549 - protein, 8 aa, synthetic construct
ISPYGGST

SEQ ID NO: 550 - protein, 11 aa, synthetic construct
ARRHWPGGFDY

SEQ ID NO: 551 - protein, 6 aa, synthetic construct
QDVSTA

SEQ ID NO: 552 - protein, 9 aa, synthetic construct
QQYLYHPAT

SEQ ID NO: 553 - protein, 7 aa, synthetic construct
GFTFSDS

SEQ ID NO: 554 - protein, 6 aa, synthetic construct
SPYGGS

SEQ ID NO: 555 - protein, 9 aa, synthetic construct
RHWPGGFDY

SEQ ID NO: 556 - protein, 11 aa, synthetic construct
RASQDVSTAVA

SEQ ID NO: 557 - protein, 7 aa, synthetic construct
SASFLYS

SEQ ID NO: 558 - protein, 9 aa, synthetic construct
QQYLYHPAT

SEQ ID NO: 559 - protein, 120 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 560 - protein, 113 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQP

SEQ ID NO: 561 - protein, 5 aa, synthetic construct
SYIMM

SEQ ID NO: 562 - protein, 17 aa, synthetic construct
SIYPSGGITFYADTVKG

SEQ ID NO: 563 - protein, 11 aa, synthetic construct
IKLGTVTTVDY

SEQ ID NO: 564 - protein, 14 aa, synthetic construct
TGTSSDVGGYNYVS

SEQ ID NO: 565 - protein, 7 aa, synthetic construct
DVSNRPS

SEQ ID NO: 566 - protein, 10 aa, synthetic construct
SSYTSSSTRV

SEQ ID NO: 567 - protein, 8 aa, synthetic construct
GFTFSSYI

SEQ ID NO: 568 - protein, 8 aa, synthetic construct
IYPSGGIT

SEQ ID NO: 569 - protein, 13 aa, synthetic construct
ARIKLGTVTTVDY

SEQ ID NO: 570 - protein, 10 aa, synthetic construct
SSYTSSSTRV

SEQ ID NO: 571 - protein, 10 aa, synthetic construct
SSYTSSSTRV

SEQ ID NO: 572 - protein, 7 aa, synthetic construct
GFTFSSY

SEQ ID NO: 573 - protein, 6 aa, synthetic construct
YPSGGI

SEQ ID NO: 574 - protein, 11 aa, synthetic construct
IKLGTVTTVDY

SEQ ID NO: 575 - protein, 14 aa, synthetic construct
TGTSSDVGGYNYVS

SEQ ID NO: 576 - protein, 7 aa, synthetic construct
DVSNRPS

SEQ ID NO: 577 - protein, 10 aa, synthetic construct
SSYTSSSTRV

SEQ ID NO: 578 - protein, 121 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 579 - protein, 111 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTV

SEQ ID NO: 580 - protein, 5 aa, synthetic construct
RYWMS

SEQ ID NO: 581 - protein, 17 aa, synthetic construct
NIKQDGSEKYYVDSVKG

SEQ ID NO: 582 - protein, 12 aa, synthetic construct
EGGWFGELAFDY

SEQ ID NO: 583 - protein, 12 aa, synthetic construct
RASQRVSSSYLA

SEQ ID NO: 584 - protein, 7 aa, synthetic construct
DASSRAT

SEQ ID NO: 585 - protein, 9 aa, synthetic construct
QQYGSLPWT

SEQ ID NO: 586 - protein, 8 aa, synthetic construct
GFTFSRYW

SEQ ID NO: 587 - protein, 8 aa, synthetic construct
IKQDGSEK

SEQ ID NO: 588 - protein, 14 aa, synthetic construct
AREGGWFGELAFDY

SEQ ID NO: 589 - protein, 7 aa, synthetic construct
QRVSSSY

SEQ ID NO: 590 - protein, 9 aa, synthetic construct
QQYGSLPWT

SEQ ID NO: 591 - protein, 7 aa, synthetic construct
GFTFSRY

SEQ ID NO: 592 - protein, 6 aa, synthetic construct
KQDGSE

SEQ ID NO: 593 - protein, 12 aa, synthetic construct
EGGWFGELAFDY

SEQ ID NO: 594 - protein, 12 aa, synthetic construct
RASQRVSSSYLA

SEQ ID NO: 595 - protein, 7 aa, synthetic construct
DASSRAT

SEQ ID NO: 596 - protein, 9 aa, synthetic construct
QQYGSLPWT

SEQ ID NO: 597 - protein, 636 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 598 - protein, 636 aa, synthetic construct
DKTHTCPPCPAPEAARAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGSDKTHTCPPCPAPEAARAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 599 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 600 - protein, 330 aa, synthetic construct [sequence omitted]

SEQ ID NO: 601 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 602 - protein, 377 aa, synthetic construct
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 603 - protein, 107 aa, synthetic construct [sequence omitted]

SEQ ID NO: 604 - protein, 106 aa, synthetic construct
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 605 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 606 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 607 - protein, 106 aa, synthetic construct
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 608 - protein, 106 aa, synthetic construct
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS

(19) United States
(12) Patent Application Publication
(10) Pub. No.: US 2026/0209370 A1
(43) Pub. Date: Jul. 23, 2026

(54) TUMOR NECROSIS FACTOR RECEPTOR AGONISTIC MBS

(71) Applicant: Radiant Biotherapeutics Inc., Montreal (CA)

(72) Inventors: Joanne Hulme, Toronto (CA); Peter Bayliss, Toronto (CA); Abilasha Rao, Toronto (CA)

(21) Appl. No.: 19/140,336

(22) PCT Filed: Dec. 18, 2023
(86) PCT No.: PCT/CA2023/051692

§ 371 Date: Jun. 17, 2025

Related U.S. Application Data
(60) Provisional application No. 63/433,477, filed on Dec. 18, 2022.

(51) Int. Cl. C07K 16/28; A61K 39/00; A61P 35/00; C07K 14/47

(52) U.S. Cl. CPC C07K 16/2878; A61P 35/00; C07K 14/47; C07K 16/2827; C07K 16/2863; A61K 2039/505; C07K 2317/31; C07K 2317/55; C07K 2319/20

(57) ABSTRACT

Fusion polypeptides comprising: (1) a TNF receptor-binding moiety and (2) a nanocage monomer or subunit thereof; self-assembled polypeptide complexes comprising such fusion polypeptides. Self-assembled polypeptide complexes comprising Fc fusion polypeptides and fusion polypeptides disclosed herein and/or tumor-binding fusion polypeptides, and related compositions and methods.

### CROSS-REFERENCE TO RELATED APPLICATIONS

This application claims the benefit of and priority to U.S. Provisional Patent Application No. 63/433,477 (filed on Dec. 18, 2022), the disclosure of which is incorporated by reference herein in their entirety for all purposes.

### SEQUENCE LISTING

The present specification makes reference to a Sequence listing which has been submitted electronically in XML format and is hereby incorporated by reference in its entirety. Said XML file, created on Dec. 13, 2023, is named RBT-054WO_SL.xml, and is 530 kb in size.

### BACKGROUND

The tumor necrosis factor (TNF) receptor superfamily includes various receptors that become activated upon ligand binding and multimerization of the receptors at the cell surface is critical for the initiation of downstream signaling. Depending on the type of TNF receptor, activation may induce a downstream signal involved in a variety of cellular processes. When activated, certain members of the TNF receptor family can provide co-stimulatory signals for T-cells such as tumor-antigen specific T-cells. Thus, these TNF receptor family members have been attractive targets for immunotherapies. However, therapies using this strategy face a trade-off between efficacy and safety. For example, urelumab, an antibody that binds to the TNF receptor family member 4-1BB, exhibits clinical efficacy and strong agonistic activity at 4-1BB. However, this agonistic activity is dependent on Fc crosslinking and is associated with liver toxicity. By contrast, utomilumab, another 4-1BB antibody, exhibits a clean liver profile but much weaker agonism which is also dependent on Fc crosslinking.

Thus, there is a need for improved therapeutics that exhibit both therapeutic efficacy and a desirable safety profile.

### SUMMARY

The present invention addresses this need with the provision of self-assembled polypeptide complexes displaying TNF receptor-binding moieties. These complexes exhibit robust agonism of TNF receptors, independent of Fc crosslinking or Fc effector functions. Thus, these complexes may exhibit improve safety profiles without sacrificing efficacy. Additionally, these complexes may provide a therapeutic modality for treating immunologically cold tumors, which have low levels or no infiltration of Fcγ receptor-expressing cells. Also disclosed are related fusion proteins, complexes, compositions, and methods.

In one aspect, provided are fusion polypeptides comprising: (1) a tumor necrosis factor (TNF) receptor-binding moiety and (2) a nanocage monomer or subunit thereof, wherein the TNF receptor-binding moiety is not capable of binding to death receptor 4 (DR4) or death receptor 5 (DR5).

In some embodiments, the TNF receptor-binding moiety comprises an antibody or antigen-binding fragment thereof.

In some embodiments, the antibody or antigen-binding fragment thereof comprises an immunoglobulin heavy chain variable region (V_{H}) and an immunoglobulin light chain variable region (V_{L} or V_{K}).

In some embodiments, the antibody or antigen-binding fragment thereof comprises a Fab fragment.

In some embodiments, the Fab fragment is a single-chain Fab fragment (scFab).

In some embodiments, the TNF receptor-binding moiety is not capable of binding to a TNF receptor which comprises a cytoplasmic tail which comprises a death domain.

In some embodiments, the TNF-receptor-binding moiety is capable of binding to a TNF receptor that is expressed on a hematopoietic cell.

In some embodiments, the TNF receptor is selected from the group consisting of 4-1BB (also known as CD137), OX40 (also known as CD134), CD40, herpes-virus entry mediator (HVEM), CD30, CD27 (also known as TNFRSF7), GITR (also known as TNFRSF18), and TNFR2.

In some embodiments, the TNF receptor is 4-1BB or OX40.

In some embodiments, the TNF receptor-binding moiety comprises an antibody fragment comprising a heavy chain variable region (V_{H}) and light chain variable region (V_{L} or V_{K}), wherein the CDRs of the V_{H} and the V_{L} or V_{K} (CDR-H1, CDR-H2, CDR-H3, CDR-L1, CDR-L2, and CDR-L3) have sequences which each differ by at most two amino acids from the CDRs of a 4-1BB or OX40 antibody.

In some embodiments, the CDRs of the V_{H} and the V_{L} or V_{K} have sequences which are identical to those of the heavy and light chain CDRs of a 4-1BB or OX40 antibody, except for one or two amino acid substitutions total across all six CDRs.

In some embodiments, the CDRs of the V_{H} and the V_{L} or V_{K} are identical to those of the heavy and light chain CDRs of a 4-1BB or OX40 antibody.

In some embodiments, the TNF receptor-binding moiety comprises an antibody fragment comprising heavy and light chain variable regions with sequences that have at least 85% identity to those of the heavy and light chain variable regions of a 4-1BB or OX40 antibody.

In some embodiments, the nanocage monomer is a ferritin monomer or a subunit thereof.

In some embodiments, the ferritin monomer is a human ferritin monomer.

In some embodiments, the ferritin monomer is a ferritin light chain.

In some embodiments, the TNF receptor-binding moiety is linked via an amino acid linker to the nanocage monomer or subunit thereof.

In some embodiments, the TNF receptor-binding moiety is linked via the N-terminus of the nanocage monomer or subunit thereof.

In one aspect, provided are self-assembled polypeptide complexes comprising: (a) a plurality of TNF receptor-binding fusion polypeptides, each TNF receptor-binding fusion polypeptide being a fusion polypeptide of as disclosed herein; and (b) a plurality of Fc fusion polypeptides, each Fc fusion polypeptide comprising (1) an Fc polypeptide and (2) a nanocage monomer or subunit thereof.

In some embodiments, the self-assembled polypeptide complex further comprises (c) a plurality of tumor-binding fusion polypeptides, each tumor-binding fusion polypeptide comprising (1) tumor-binding moiety and (2) a nanocage monomer or subunit thereof.

In some embodiments, within each Fc fusion polypeptide, the Fc polypeptide is linked via an amino acid linker to the nanocage monomer or subunit thereof.

In some embodiments, the Fc polypeptide is linked via the N-terminus of the nanocage monomer or subunit thereof.

In some embodiments, the Fc polypeptide is linked via the C-terminus of the nanocage monomer or subunit thereof.

In some embodiments, with each tumor-binding fusion polypeptide, the tumor-binding moiety is linked via an amino acid linker to the nanocage monomer or subunit thereof.

In some embodiments, the tumor-binding moiety is linked via the N-terminus of the nanocage monomer or subunit thereof.

In some embodiments, the tumor-binding moiety comprises an antibody or antigen-binding fragment thereof.

In some embodiments, the antibody or antigen-binding fragment thereof comprises an immunoglobulin heavy chain variable region (V_{H}) and an immunoglobulin light chain variable region (V_{L} or V_{K}).

In some embodiments, the antibody or antigen-binding fragment thereof comprises a Fab fragment.

In some embodiments, the Fab fragment is a single-chain Fab fragment (scFab).

In some embodiments, the tumor-binding moiety is a PD-L1-binding moiety, an EGFR-binding moiety, or a B7-H3-binding moiety.

In some embodiments, the tumor-binding moiety comprises an antibody fragment comprising a heavy chain variable region (V_{H}) and light chain variable region (V_{L} or V_{K}), wherein the CDRs of the V_{H} and the V_{L} or V_{K} (CDR-H1, CDR-H2, CDR-H3, CDR-L1, CDR-L2, and CDR-L3) have sequences which each differ by at most two amino acids from the CDRs of a PD-L1 binding moiety, an EGFR-binding moiety, or a B7-H3-binding moiety antibody.

In some embodiments, the CDRs of the V_{H} and the V_{L} or V_{K} have sequences which are identical to those of the heavy and light chain CDRs of a PD-L1, an EGFR-binding moiety, or a B7-H3-binding moiety antibody, except for one or two amino acid substitutions total across all six CDRs.

In some embodiments, the CDRs of the V_{H} and the V_{L} or V_{K} are identical to those of the heavy and light chain CDRs of a PD-L1, EGFR, or B7-H3 antibody.

In some embodiments, the tumor-binding moiety comprises an antibody fragment comprising heavy and light chain variable regions with sequences that have at least 85% identity to those of the heavy and light chain variable regions of a PD-L1, EGFR, or B7-H3 antibody.

In some embodiments, the nanocage monomers within the Fc fusion polypeptide and within TNF receptor-binding fusion polypeptide and/or the tumor-binding fusion polypeptide are each a ferritin monomer or a subunit thereof.

In some embodiments, the ferritin monomer is a human ferritin monomer.

In some embodiments, the ferritin monomer is a ferritin light chain.

In some embodiments, the self-assembled polypeptide complex does not comprise any ferritin heavy chains or subunits of ferritin heavy chains.

In some embodiments, the self-assembled polypeptide complex does not comprise any iron-binding moieties.

In one aspect, provided are pharmaceutical compositions comprising a self-assembled polypeptide complex as disclosed herein and a pharmaceutically acceptable excipient.

In one aspect, provided are uses of a self-assembled polypeptide complex or a pharmaceutical composition as disclosed herein to treat, ameliorate, or prevent a disease or condition in a subject.

In one aspect, provided are methods of treating, ameliorating, or preventing a disease or condition, the method comprising administering to a subject a self-assembled polypeptide complex or a pharmaceutical composition as disclosed herein.

In some embodiments, the subject is a mammal, e.g., a human.

In some embodiments, the disease or condition is cancer.

In some embodiments, the subject has an immunologically cold tumor.

In some embodiments, the subject has a tumor, and said step of administering results in regression of the tumor. In some embodiments, the step of administering results in complete regression of the tumor.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a diagrammatic representation of human ferritin light chain (hFTL) and example N-half ferritin (N-hFTL) and C-half ferritin (C-hFTL) molecules.

FIGS. 1B and 1C are diagrammatic representations of sets of fusion polypeptides that together may form exemplary Multabodies (MBs) of the disclosure.

FIG. 2 is a schematic depicting the various extracellular cysteine-rich domains (CRDs) of 4-1BB and their respective distances from the cell membrane.

FIGS. 3A and 3B are schematics that depict an in vitro 4-1BB reporter assay. Luciferase expression is induced by downstream signals upon activation (clustering) of 4-1BB at the surface of effector cells. The assay can be performed without (FIG. 3A) or with (FIG. 3B) FcγRIIb CHO-K1 cells to assess Fc-independent or Fc-dependent crosslinking, respectively.

FIGS. 4A and 4B are graphs showing the fold induction (based on luciferase signal) of 4-1BB activation (y-axis) plotted against concentration of urelumab (x-axis). FIG. 4A depicts the results from assays conducted without FcγRIIb CHO-K1 cells (and therefore without Fc crosslinking), and FIG. 4B depicts the results from assays conducted in the presence of FcγRIIb CHO-K1 cells (and therefore with Fc crosslinking). Also shown are levels induced by human 4-1BB ligand (h-4-1BBL) and a class-matched IgG control (not capable of binding to 4-1BB).

FIGS. 4C and 4D are graphs showing the fold induction (based on luciferase signal) of 4-1BB activation (y-axis) plotted against concentration of utomilumab (IgG1 or IgG2) (x-axis). FIG. 4C depicts the results from assays conducted without FcγRIIb CHO-K1 cells (and therefore without Fc crosslinking), and FIG. 4D depicts the results from assays conducted in the presence of FcγRIIb CHO-K1 cells (and therefore with Fc crosslinking). Also shown are levels induced by human 4-1BB ligand (h-4-1BBL) and a class-matched IgG control (not capable of binding to 4-1BB).

FIGS. 5A and 5B are graphs showing the fold induction (based on luciferase signal) of 4-1BB activation (y-axis) plotted against concentration of effector silent α-4-1BB (AG10058; Fab #3) MBs (x-axis). FIG. 5A depicts the results from assays conducted without FcγRIIb CHO-K1 cells (and therefore without Fc crosslinking), and FIG. 5B depicts the results from assays conducted in the presence of FcγRIIb CHO-K1 cells (and therefore with Fc crosslinking). Also shown are levels induced by human 4-1BB ligand (h-41BBL), urelumab IgG, AG10058 IgG (IgG4), a MB negative control (which does not display any α-4-1BB Fabs), and a class-matched IgG negative control (not capable of binding to 4-1BB).

FIG. 6 is a schematic depicting the various extracellular cysteine-rich domains (CRDs) of 4-1BB and their respective distances from the cell membrane. Also shown are the epitope bins identified by competition assays performed with the listed α-4-1BB IgG antibodies, further categorized as “non ligand blocking” or “ligand blocking”.

FIGS. 7A and 7B are graphs showing the fold induction (based on luciferase signal) of 4-1BB activation (y-axis) plotted against concentration of urelumab MB and the parent IgG4 urelumab (x-axis). FIG. 7A depicts the results from assays conducted without FcγRIIb CHO-K1 cells (and therefore without Fc crosslinking), and FIG. 7B depicts the results from assays conducted in the presence of FcγRIIb CHO-K1 cells (and therefore with Fc crosslinking). Also shown are levels induced by human 4-1BB ligand (h-4-1BBL), and a class-matched IgG control and a corresponding negative control MB (not capable of binding to 4-1BB).

FIGS. 7C and 7D are graphs showing the fold induction (based on luciferase signal) of 4-1BB activation (y-axis) plotted against concentration of effector silent α-4-1BB (NM21; Fab #1) MB and the parent IgG1 (x-axis). FIG. 7C depicts the results from assays conducted without FcγRIIb CHO-K1 cells (and therefore without Fc crosslinking), and FIG. 7D depicts the results from assays conducted in the presence of FcγRIIb CHO-K1 cells (and therefore with Fc crosslinking). Also shown are levels induced by human 4-1BB ligand (h-4-1BBL), and a class-matched IgG control and a corresponding negative control MB (not capable of binding to 4-1BB).

FIGS. 7E and 7F are graphs showing the fold induction (based on luciferase signal) of 4-1BB activation (y-axis) plotted against concentration of effector silent α-4-1BB (A1; Fab #2) MB and the parent IgG1 (x-axis). FIG. 7E depicts the results from assays conducted without FcγRIIb CHO-K1 cells (and therefore without Fc crosslinking), and FIG. 7F depicts the results from assays conducted in the presence of FcγRIIb CHO-K1 cells (and therefore with Fc crosslinking). Also shown are levels induced by human 4-1BB ligand (h-4-1BBL), and a class-matched IgG control and a corresponding negative control MB (not capable of binding to 4-1BB).

FIGS. 7G and 7H are graphs showing the fold induction (based on luciferase signal) of 4-1BB activation (y-axis) plotted against concentration of effector silent α-4-1BB (AG10058; Fab #3) MB and the parent IgG4 (x-axis). FIG. 7G depicts the results from assays conducted without FcγRIIb CHO-K1 cells (and therefore without Fc crosslinking), and FIG. 7H depicts the results from assays conducted in the presence of FcγRIIb CHO-K1 cells (and therefore with Fc crosslinking). Also shown are levels induced by human 4-1BB ligand (h-4-1BBL), and a class-matched IgG control and a corresponding negative control MB (not capable of binding to 4-1BB).

FIGS. 7I and 7K are graphs showing the fold induction (based on luciferase signal) of 4-1BB activation (y-axis) plotted against concentration of effector silent α-4-1BB (ABLPNB.03; Fab #6) MB and the parent IgG1 (x-axis). FIG. 7I depicts the results from assays conducted without FcγRIIb CHO-K1 cells (and therefore without Fc crosslinking), and FIG. 7K depicts the results from assays conducted in the presence of FcγRIIb CHO-K1 cells (and therefore with Fc crosslinking). Also shown are levels induced by human 4-1BB ligand (h-4-1BBL), and a class-matched IgG control and a corresponding negative control MB (not capable of binding to 4-1BB).

FIGS. 8A-8D are graphs showing changes in tumor volume in mm³ (y-axis) plotted against days post-first dose of the 4-1BB MB, the parent 4-1BB IgG, or a class-matched IgG control (x-axis). FIG. 8A depicts the average tumor volume for the three treatment groups (control IgG, 4-1BB IgG, and 4-1BB MB). FIG. 8B, FIG. 8C, and FIG. 8D depict tumor volume changes for the individual mice in the control IgG, 4-1BB IgG, and 4-1BB MB treatment groups, respectively.

FIGS. 9A-9D are graphs showing changes in tumor volume in mm³ (y-axis) plotted against days post-first dose of the 4-1BB MB, the urelumab IgG4, or a class-matched IgG control (x-axis). FIG. 9A depicts the average tumor volume for the three treatment groups (control IgG, urelumab IgG4, and 4-1BB MB). FIG. 9B, FIG. 9C, and FIG. 9D depict tumor volume changes for the individual mice in the control IgG, urelumab IgG4, and 4-1BB MB treatment groups, respectively.

FIG. 10 is a graph showing tumor volumes in mm³ (y-axis) plotted against days post-first dose of urelumab IgG4 or a class-matched IgG control (x-axis).

FIGS. 11A and 11B are microscopy images of liver histology samples from mice treated with 4-1-BB agents, stained by hematoxylin and eosin (H&E). FIG. 11A shows histology images at 4× and 10× magnification from mice treated with 4-1-BB MB, urelumab, or a class-matched IgG control at a dose of 5 mg/kg. FIG. 11B shows histology images at 5× and 10× magnification from mice treated with urelumab or a class-matched IgG control at a dose of 20 mg/kg. Arrows indicate regions with increased immune cell infiltration.

FIG. 12 is a graph showing the average concentration of 4-1BB IgG and 4-1BB MB in μg/ml (y-axis) in isolated mouse serum against days post-first dose of the treatments (x-axis).

### DETAILED DESCRIPTION

Disclosed herein are fusion polypeptides each comprising (1) a nanocage monomer or subunit thereof and (2) an antibody fragment (such as a TNF receptor-binding moiety) or an Fc polypeptide. The nanocage monomers or subunits thereof drive self-assembly of the fusion polypeptides into complexes (“Multabodies” or “MBs”) which display one or more of the aforementioned antibody fragments and Fc polypeptides.

### Definitions

The terms “about” and “approximately,” when used herein in reference to a value, are used interchangeably and refer to a value that is similar to the referenced value. In general, those skilled in the art, familiar with the context, will appreciate the relevant degree of variation encompassed by “about” or “approximately” in that context. For example, in some embodiments, the terms “about” and “approximately” may encompass a range of values that fall within 25%, 20%, 19%, 18%, 17%, 16%%, 15%, 14%, 13%, %12%, 11%, 10%, 9%, 8%, 7%, 6%, 5%, 4%, 3%, 2%, 1%, or less of the referred value.

As used herein, the terms “alter,” “altered,” “decrease,” “decreased,” “increase,” “increased,” or “reduction,” “reduced,” (e.g., in reference to certain outcomes or effects) have meanings relative to a reference level. In some embodiments, in the context of discussing mutations in an Fc chain or Fc polypeptide, the reference level is a level known or as determined with an IgG that does not contain the referenced mutation(s) in the Fc region.

As used herein, the term “antigen binding fragment” of an antibody, refers to one or more fragments of an antibody that retain the ability to specifically bind to an antigen. Examples of binding fragments encompassed within the term “antigen binding fragment” of an antibody include a Fab fragment, a F(ab′)2 fragment, a Fd fragment, a Fv fragment, a scFv fragment, a dAb fragment (Ward et al., (1989) Nature 341:544-546), and an isolated complementarity determining region (CDR). In some embodiments, an “antigen binding fragment” comprises a heavy chain variable region and a light chain variable region. These antibody fragments can be obtained using conventional techniques known to those with skill in the art, and the fragments can be screened for utility in the same manner as are intact antibodies.

As used herein, the term “binding,” unless otherwise specified, refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell). As used herein, the phrases “non-binding” or “no binding,” or similar phrases, between two entities refers to 1) a lack of detectable binding or 2) binding below a set threshold that corresponds to no binding in an appropriate assay, e.g., an in vitro binding assay such as biolayer interferometry, surface plasmon resonance, a cell binding assay such as flow cytometry, or enzyme-linked immunosorbent assay (ELISA). For example, in some embodiments, in an in vitro biolayer interferometry assay, a maximal association binding response of less than 0.1 nm after 180 seconds to a biosensor loaded with 0.8 nm of target when the test article is present at a concentration of 20 nM is classified as “non-binding.”

As used herein, the phrases “cold” or “immunologically cold,” when used in reference to a tumor or a cancer, refers to a tumor that is not responsive to checkpoint inhibition (at least in the absence of a therapeutic agent other than a checkpoint inhibitor.) Typically, an immunologically cold tumor, in the absence of a therapeutic agent, is characterized by a lack or paucity of tumor T cell infiltration. Examples of immunologically cold tumors include, without limitation, glioblastomas, ovarian cancer, prostate cancer, pancreatic cancer, and breast cancer tumors that are characterized by a lack of T cell infiltration.

The terms “ferritin” and “apoferritin” are used interchangeably herein and generally refer to a polypeptide (e.g., a ferritin chain) that is capable of assembling into a ferritin complex which typically comprises 24 protein subunits. In some embodiments, the ferritin is a human ferritin, e.g., a human ferritin light chain, e.g., a human ferritin light chain having at least 85% sequence identity to SEQ ID NO:1 or UniProt P02792. In some embodiments, the ferritin is a wild-type ferritin. For example, the ferritin may be a wild-type human ferritin.

The term “ferritin monomer,” is used herein to refer to a single chain of a ferritin that, in the presence of other ferritin chains, is capable of self-assembling into a polypeptide complex comprising a plurality of ferritin chains, e.g., 24 or more ferritin chains.

As used herein, the term “linker” is used to refer to an entity that connects two or more elements to form a multi-element agent. For example, those of ordinary skill in the art appreciate that a polypeptide (e.g., fusion polypeptide) whose structure includes two or more functional or organizational domains often includes a stretch of amino acids between such domains that links them to one another. In some embodiments, a polypeptide comprising a linker element has an overall structure of the general form S1-L-S2, wherein S1 and S2 may be the same or different and represent two domains associated with one another by the linker (L). In some embodiments, the linker is an “amino acid linker,” that is, it comprises amino acid residues, e.g., an amino acid linker may comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acid residues. In some embodiments, a linker is characterized in that it tends not to adopt a rigid three-dimensional structure, but rather provides flexibility to the polypeptide.

The term “multispecific,” as used herein, refers to the characteristic of having at least two binding sites at which at least two different binding partners, e.g., an antigen or receptor (e.g., Fc receptor), can bind. For example, a polypeptide complex that comprises at least two Fab fragments, wherein each of the two Fab fragments is capable of binding (e.g., specifically binding) to a different antigen, is “multispecific.” As an additional example, a polypeptide complex that comprises an Fc fragment (which is capable of binding (e.g., specifically binding) to an Fc receptor) and a Fab fragment (which is capable of binding (e.g., specifically binding) to an antigen) is “multispecific.”

The term “multivalent,” as used herein, refers to the characteristic of having at least two binding sites at which a binding partner, e.g., an antigen or receptor (e.g., Fc receptor), can bind. The binding partners that can bind to at least two binding sites may be the same or different.

The term “nanocage monomer,” as used herein, refers to a single chain of a polypeptide that is capable of self-assembling with other nanocage monomers to form a self-assembled polypeptide complex comprising a plurality of nanocage monomers. In some embodiments, the nanocage monomer is selected from monomers of ferritin, apoferritin, encapsulin, sulfur oxygenase reductase (SOR), lumazine synthase, pyruvate dehydrogenase, carboxysome, vault proteins, GroEL, heat shock protein, E2P coat protein, MS2 coat protein, fragments thereof, and variants thereof.

The term “polypeptide,” as used herein, generally has its art-recognized meaning of a polymer of at least three amino acids, e.g., linked to each other by peptide bonds. Those of ordinary skill in the art will appreciate that the term “polypeptide” is intended to be sufficiently general as to encompass not only polypeptides having a complete sequence recited herein, but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Polypeptides may contain L-amino acids, D-amino acids, or both and may contain any of a variety of amino acid modifications or analogs known in the art. Useful modifications include, e.g., terminal acetylation, amidation, methylation, glycosylation etc. In some embodiments, proteins may comprise natural amino acids, non-natural amino acids, synthetic amino acids, and combinations thereof.

As used herein, the term “specifically binds, “specifically binding,” “binds specifically,” or similar terms, means that a binding moiety (e.g., an antibody or an antigen-binding fragment thereof) forms a complex with an antigen that is relatively stable under physiologic conditions. Specific binding can be characterized by an equilibrium dissociation constant of 1×10⁻⁶ M or less, 1×10⁻⁷ M or less, 1×10⁻⁸ M or less, or 1×10⁻⁹ M or less (e.g., a smaller K_{D} denotes a tighter binding). Methods for determining whether two molecules specifically bind are well known in the art and include, for example, equilibrium dialysis, surface plasmon resonance, enzyme-linked immunosorbent assay, or biolayer interferometry measurements, etc. In some embodiments, “specifically binds” and similar terms refers to a characteristic of the binding moiety in that the binding moiety is capable of binding to a target antigen but does is not capable of binding to other antigens such as distantly related family members of the antigen.

The term “self-assembled,” when used in reference to a macromolecular complex (e.g., a polypeptide complex), refers to the spontaneous formation of that complex when sufficient constituents of the complex (e.g., fusion polypeptides) to be formed are present. In some embodiments, complexes self-assemble in physiological conditions, or in a buffer (e.g., a solution) that corresponds to physiological conditions.

As used herein, the term “subject” to an organism, typically a mammal (e.g., a human). In some embodiments, a subject is suffering from or susceptible to a relevant disease, disorder or condition. In some embodiments, a subject displays one or more symptoms or characteristics of a disease, disorder or condition. In some embodiments, a subject is someone with one or more features characteristic of susceptibility to or risk of a disease, disorder, or condition. In some embodiments, a subject is a patient. In some embodiments, a subject is a subject to whom diagnosis and/or therapy is and/or has been administered.

As used herein, the term “treatment” (also “treat” or “treating”) refers to any administration of a therapy that partially or completely alleviates, ameliorates, relieves, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively, or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

### A. Fusion Polypeptides

In many embodiments, fusion polypeptides compatible with compositions and methods disclosed herein generally comprise (1) a nanocage monomer (or subunit thereof) as described herein and (2) tumor necrosis factor (TNF) receptor-binding moiety or an Fc polypeptide, which may be linked via a linker, such as a linker described herein.

### 1. Nanocage Monomers and Subunits Thereof

In some embodiments, the nanocage monomer is a ferritin monomer.

The term “ferritin monomer,” is used herein to refer to a single chain of a ferritin that, in the presence of other ferritin chains, is capable of self-assembling into a polypeptide complex comprising a plurality of ferritin chains, e.g., 24 or more ferritin chains. In some embodiments, the ferritin monomer is a ferritin light chain. In some embodiments, the ferritin monomer does not include a ferritin heavy chain or other ferritin components capable of binding to iron or capable of ferroxidase activity.

In some embodiments, each fusion polypeptide within the self-assembled polypeptide complex comprises a ferritin light chain or a subunit of a ferritin light chain. In these embodiments, the self-assembled polypeptide complex does not comprise any ferritin heavy chains or subunits of ferritin heavy chains. In such embodiments, the self-assembled polypeptide complex does not comprise and is not associated with Fe³⁺, an iron ion normally carried by ferritin complexes which comprise the ferritin heavy chain.

In some embodiments, the ferritin monomer is a human ferritin chain, e.g., a human ferritin light chain, e.g., a human ferritin light chain having the sequence of at least residues 2-175 of SEQ ID NO:1.

In some embodiments, the ferritin monomer is a full-length human ferritin light chain. As used herein, a “full-length ferritin light chain” refers to a ferritin polypeptide that is capable of forming a four-helix bundle of two couples of anti-parallel α-helices connected by a loop and further comprising a C-terminal α-helix. In some embodiments, a full-length ferritin light chain comprises at least 85%, at least 90%, at least 95%, or 100% of the length of an amino acid sequence of SEQ ID NO: 1. In some embodiments, a full-length ferritin light chain has an amino acid sequence which has at least 85%, at least 90%, or at least 95% sequence identity to the SEQ ID NO: 1. In some embodiments, a full-length ferritin light chain has an amino acid sequence of SEQ ID NO:1. In some embodiments, the full-length ferritin light chain is a single contiguous ferritin polypeptide.

A “subunit” of a ferritin monomer refers to a portion of a ferritin monomer that is capable of spontaneously associating with another, distinct subunit of a ferritin monomer, so that the subunits together form a ferritin monomer, which ferritin monomer, in turn, is capable of self-assembling with other ferritin monomers to form a polypeptide complex.

In some embodiments, the ferritin monomer subunit comprises approximately half of a ferritin monomer. As used herein, the term “N-half ferritin” refers to approximately half of a ferritin chain, which half comprises the N-terminus of the ferritin chain. As used herein, the term “C-half ferritin” refers to approximately half a ferritin chain, which half comprises the C-terminus of the ferritin chain. The exact point at which a ferritin chain may be divided to form the N-half ferritin and the C-half ferritin may vary depending on the embodiment. In the context of ferritin monomer subunits based on human ferritin light chain, for example, the halves may be divided at a point that corresponds to a position between about position 75 to about position 100 of SEQ ID NO:1 (or a substantial portion thereof). For example, in some embodiments, an N-half ferritin based on a human ferritin light chain has an amino acid sequence corresponding to residues 1-95 of SEQ ID NO:1 (or a substantial portion thereof, e.g., residues 2-95 of SEQ ID NO:1), and a C-half ferritin based on a human ferritin light chain has an amino acid sequence corresponding to residues 96-175 of SEQ ID NO:1 (or a substantial portion thereof).

In some embodiments, the halves are divided at a point that corresponds to a position between about position 85 to about position 92 of SEQ ID NO: 1. For example, in some embodiments, an N-half ferritin based on a human ferritin light chain has an amino acid sequence corresponding to residues 1-90 of SEQ ID NO:1 (or a substantial portion thereof, e.g., residues 2-90 of SEQ ID NO:1), and a C-half ferritin based on a human ferritin light chain has an amino acid sequence corresponding to residues 91-175 of SEQ ID NO:1 (or a substantial portion thereof.

### 2. Binding Moieties

Binding moieties (e.g., TNF receptor-binding moieties) typically comprise an antibody fragment.

In some embodiments, the antibody fragment is a Fab. In some embodiments, the antibody fragment is a single-chain Fab (scFab); for example, a fusion polypeptide comprising both the heavy and light chains of a Fab, optionally linked by a linker (e.g., amino acid linker as disclosed herein) is used.

In certain embodiments, the antibody fragment comprises a heavy chain variable region (e.g., a V_{H}). In certain embodiments, the antibody fragment comprises a heavy chain variable domain (e.g., V_{H}) and a light chain variable domain (e.g., a V_{L} or V_{K}). In certain embodiments, the antibody fragment comprises a Fab which comprises a heavy chain variable domain (e.g., V_{H}) and a light chain variable domain (e.g., a V_{L} or V_{K}).

In certain embodiments, the antibody fragment does not comprise any domains from the Fc region, e.g., does not comprise any constant heavy (CH2 or CH3) domains. In some embodiments, the antibody fragment is an antibody fragment of, or derived from, a fully human or humanized antibody. In some embodiments, the antibody fragment is an antibody fragment of, or derived from, a chimeric antibody. The antibody from which the antibody fragment is obtained or derived can be of any of a variety of antibody classes, including, e.g., an IgG1 antibody, an IgG2 antibody, or an IgG4 antibody. In some embodiments, the antibody fragment is obtained or derived from an agonistic antibody, e.g., an agonistic humanized antibody.

In embodiments where multiple types of fusion polypeptides having antibody fragments are used, the antibody fragments in the various types of fusion polypeptides may be capable of binding to the same epitope on a given antigen (e.g., an antigen on a TNF receptor or a tumor-associated antigen), capable of binding to epitopes that are distinct and non-overlapping on an antigen, or capable of binding to epitopes that are distinct but overlapping on the same antigen.

### A. Tumor Necrosis Factor (TNF) Receptor-Binding Moieties

Tumor necrosis factor (TNF) receptor-binding moieties are generally capable of binding (e.g., specifically binding) to a member of the TNF receptor superfamily. In some embodiments, the TNF-receptor-binding moiety is capable of binding (e.g., specifically binding) to a TNF receptor which comprises a cytoplasmic tail which does not comprise a death domain. In some embodiments, the TNF-receptor-binding moiety is capable of binding (e.g., specifically binding) to a TNF receptor that is not DR4 (death receptor 4) or DR5 (death receptor 5).

In some embodiments, the TNF-receptor-binding moiety is capable of binding (e.g., specifically binding) to a TNF receptor that is expressed on a hematopoietic cell (such as a lymphoid cell (e.g., T cell, B cell, or plasmacytoid dendritic cell) or a myeloid cell (e.g., macrophage, microglial cell, or interstitial dendritic cell)). For example, the TNF receptor may be selected from the group consisting of 4-1BB (also known as CD137), OX40 (also known as CD134), CD40, herpes-virus entry mediator (HVEM), CD30, CD27 (also known as TNFRSF7), GITR (also known as TNFRSF18), and TNFR2.

In some embodiments, the TNF receptor is expressed on a T cell.

In some embodiments, the TNF-receptor-binding moiety is capable of binding (e.g., specifically binding) to 4-1BB or OX40.

In some embodiments, the TNF-receptor-binding moiety comprises an antibody fragment comprising a heavy chain variable region (V_{H}) and light chain variable region (V_{L} or V_{K}), wherein the CDRs of the V_{H} and the V_{L} or V_{K} (CDR-H1, CDR-H2, CDR-H3, CDR-L1, CDR-L2, and CDR-L3) have sequences which are similar to (e.g., each having at most one or two amino acids differing from) those of the heavy and light chain CDRs of a 4-1BB or OX40 antibody (e.g., a human or humanized 4-1BB or OX40 antibody).

In some embodiments, the TNF-receptor-binding moiety is an antibody fragment comprising a heavy chain variable region (V_{H}) and light chain variable region (V_{L} or V_{K}), wherein the CDRs of the V_{H} and the V_{L} or V_{K} have sequences which are identical to those of the heavy and light chain CDRs of a 4-1BB or OX40 antibody (e.g., a human or humanized 4-1BB or OX40 antibody), except for one or two amino acid substitutions total across all six CDRs.

In some embodiments, the TNF-receptor-binding moiety is an antibody fragment comprising a heavy chain variable region (V_{H}) and light chain variable region (V_{L} or V_{K}), wherein the CDRs of the V_{H} and the V_{L} or V_{K} are identical to those of the heavy and light chain CDRs of a 4-1BB or OX40 antibody (e.g., a human or humanized 4-1BB or OX40 antibody).

In some embodiments, the TNF-receptor-binding moiety is an antibody fragment comprising heavy and light chain variable regions (e.g., V_{H} and V_{L} or V_{K}) with sequences that have at least 85%, at least 87.5%, at least 90%, at least 91%, at least 92%, at least 93%, at least 94%, at least 95%, at least 96%, at least 97%, at least 98% or at least 99% identity to those of the heavy and light chain variable regions of a 4-1BB or OX40 antibody (e.g., a human or humanized 4-1BB or OX40 antibody). In some embodiments, the TNF-receptor-binding moiety is an antibody fragment comprising heavy and light chain variable regions (e.g., V_{H} and V_{L} or V_{K}) with sequences identical to those of the heavy and light chain variable regions of a 4-1BB or OX40 antibody (e.g., a human or humanized 4-1BB or OX40 antibody).

Non-limiting examples of 4-1BB antibodies include urelumab and utomilumab, and additional antibodies whose heavy chain variable region, light chain variable region, and CDR sequences are depicted in Tables TA-1Q. (The sequences for the 4-1BB-binding arm of acasunlimab, a bispecific antibody, is also included.)

Non-limiting examples of OX40 antibodies include cudarolumab, ivuxolimab, revdofilimab, tavolimab, vonlerolizumab (pogalizumab), and additional antibodies whose heavy chain variable region, light chain variable region, and CDR sequences are depicted in Tables 2A-2K.

TABLE 1A: 4-1BB Antibody Sequences (NM21)

| Heavy chain variable region | | Light chain variable region | |
|---|---|---|---|
| EVQLVESGGGLVQPGGSLRLSCAASGFSFSAN | | DIQMTQSPSSLSASVGDRVTITCQASQSIS | |
| YYPCWVRQAPGKGLEWIGCIYGGSSDITYDAN | | NRLAWYQQKPGKAPKLLIYSASTLASGVPS | |
| WTKGRFTISRDNSKNTVYLQMNSLRAEDTAVY | | RFSGSGSGTDFTLTISSLQPEDFATYYCQS | |
| YCARSAWYSGWGGDLWGQGTLVTVSS | | TYYGNDGNAFGTGTKVTVLG | |
| (SEQ ID NO: 8) | | (SEQ ID NO: 9) | |
| CDR Sequences (Kabat) | | CDR Sequences (Kabat) | |
| CDR-H1 | ANYYPC | CDR-L1 | QASQSISNRLA |
| | (SEQ ID NO: 10) | | (SEQ ID NO: 13) |
| CDR-H2 | CIYGGSSDITYDANWTKG | CDR-L2 | SASTLAS |
| | (SEQ ID NO: 11) | | (SEQ ID NO: 14) |
| CDR-H3 | SAWYSGWGGDL | CDR-L3 | QSTYYGNDGNA |
| | (SEQ ID NO: 12) | | (SEQ ID NO: 15) |
| CDR Sequences (IMGT) | | CDR Sequences (IMGT) | |
| CDR-H1 | GFSFSANYY | CDR-L1 | QSISNR |
| | (SEQ ID NO: 16) | | (SEQ ID NO: 19) |
| CDR-H2 | IYGGSSDIT | CDR-L2 | SA |
| | (SEQ ID NO: 17) | CDR-L3 | QSTYYGNDGNA |
| CDR-H3 | ARSAWYSGWGGDL | | (SEQ ID NO: 20) |
| | (SEQ ID NO: 18) | | |
| CDR Sequences (Chothia) | | CDR Sequences (Chothia) | |
| CDR-H1 | GFSFSANY | CDR-L1 | QASQSISNRLA |
| | (SEQ ID NO: 21) | | (SEQ ID NO: 24) |
| CDR-H2 | YGGSSDI | CDR-L2 | SASTLAS |
| | (SEQ ID NO: 22) | | (SEQ ID NO: 25) |
| CDR-H3 | SAWYSGWGGDL | CDR-L3 | QSTYYGNDGNA |
| | (SEQ ID NO: 23) | | (SEQ ID NO: 26) |

1B: 4-1BB Antibody Sequences (clone A1)

| Heavy chain variable region | | Light chain variable region | |
|---|---|---|---|
| QVQLVQSGAEVKKPGSSVKVSCKASGGTFSSY | | AIRMTQSPPSLSASVGDRVTITCQASQDIG | |
| AISWVRQAPGQGLEWMGGIIPIFGTANYAQKF | | NSLGWYQQKPGKAPKLVIFDASDLETGVPS | |
| QGRVTITADESTSTAYMELSSLRSEDTAVYYC | | RFSGSGSGTDFSLTISSLQPEDFATYYCQQ | |
| ARDLMTTAPGTYFDLWGRGTLVTVSS | | GNSFPLTFGQGTRLEIK | |
| (SEQ ID NO: 27) | | (SEQ ID NO: 28) | |
| CDR Sequences (Kabat) | | CDR Sequences (Kabat) | |
| CDR-H1 | SYAIS | CDR-L1 | QASQDIGNSLG |
| | (SEQ ID NO: 29) | | (SEQ ID NO: 32) |
| CDR-H2 | GIIPIFGTANYAQKFQG | CDR-L2 | DASDLET |
| | (SEQ ID NO: 30) | | (SEQ ID NO: 33) |
| CDR-H3 | DLMTTAPGTYFDL | CDR-L3 | QQGNSFPLT |
| | (SEQ ID NO: 31) | | (SEQ ID NO: 34) |
| CDR Sequences (IMGT) | | CDR Sequences (IMGT) | |
| CDR-H1 | GGTFSSYA | CDR-L1 | QDIGNS |
| | (SEQ ID NO: 35) | | (SEQ ID NO: 38) |
| CDR-H2 | IIPIFGTA | CDR-L2 | DA |
| | (SEQ ID NO: 36) | CDR-L3 | QQGNSFPLT |
| CDR-H3 | ARDLMTTAPGTYFDL | | (SEQ ID NO: 39) |
| | (SEQ ID NO: 37) | | |
| CDR Sequences (Chothia) | | CDR Sequences (Chothia) | |
| CDR-H1 | GGTFSSY | CDR-L1 | QASQDIGNSLG |
| | (SEQ ID NO: 40) | | (SEQ ID NO: 43) |
| CDR-H2 | IPIFGT | CDR-L2 | DASDLET |
| | (SEQ ID NO: 41) | | (SEQ ID NO: 44) |
| CDR-H3 | DLMTTAPGTYFDL | CDR-L3 | QQGNSFPLT |
| | (SEQ ID NO: 42) | | (SEQ ID NO: 45) |

TABLE 1C
4-1BB Antibody Sequences (clone ABLPNB.01)

| Heavy chain variable region | | Light chain variable region | |
|---|---|---|---|
| EVQLLESGGGLVQPGGSLRLSCAASGFTFSSY | | QSVLTQPPSASGTPGRRVTISCSGSSSNIG | |
| DMSWVRQAPGKCLEWVSWISYSGGSIYYADSV | | NNYVTWYQQLPGTAPKLLIYADSHRPSGVP | |
| KGRFTISRDNSKNTLYLQMNSLRAEDTAVYYC | | DRFSGSKSGTSASLAISGLRSEDEADYYCA | |
| ARDGQRNSMREFDYWGQGTLVTVSS | | TWDYSLSGYVFGCGTKLTVL | |
| (SEQ ID NO: 46) | | (SEQ ID NO: 47) | |
| CDR Sequences (Kabat) | | CDR Sequences (Kabat) | |
| CDR-H1 | SYDMS | CDR-L1 | SGSSSNIGNNYVT |
| | (SEQ ID NO: 48) | | (SEQ ID NO: 51) |
| CDR-H2 | WISYSGGSIYYADSVKG | CDR-L2 | ADSHRPS |
| | (SEQ ID NO: 49) | | (SEQ ID NO: 52) |
| CDR-H3 | DGQRNSMREFDY | CDR-L3 | ATWDYSLSGYV |
| | (SEQ ID NO: 50) | | (SEQ ID NO: 53) |
| CDR Sequences (IMGT) | | CDR Sequences (IMGT) | |
| CDR-H1 | GFTFSSYD | CDR-L1 | SSNIGNNY |
| | (SEQ ID NO: 54) | | (SEQ ID NO: 57) |
| CDR-H2 | ISYSGGSI | CDR-L2 | AD |
| | (SEQ ID NO: 55) | CDR-L3 | ATWDYSLSGYV |
| CDR-H3 | ARDGQRNSMREFDY | | (SEQ ID NO: 58) |
| | (SEQ ID NO: 56) | | |
| CDR Sequences (Chothia) | | CDR Sequences (Chothia) | |
| CDR-H1 | GFTFSSY | CDR-L1 | SGSSSNIGNNYVT |
| | (SEQ ID NO: 59) | | (SEQ ID NO: 62) |
| CDR-H2 | SYSGGS | CDR-L2 | ADSHRPS |
| | (SEQ ID NO: 60) | | (SEQ ID NO: 63) |
| CDR-H3 | DGQRNSMREFDY | CDR-L3 | ATWDYSLSGYV |
| | (SEQ ID NO: 61) | | (SEQ ID NO: 64) |

TABLE 1D
4-1BB Antibody Sequences (clone ABLPNB.03)

| Heavy chain variable region | | Light chain variable region | |
|---|---|---|---|
| EVQLLESGGGLVQPGGSLRLSCAASGFTFSGY | | QSVLTQPPSASGTPGRRVTISCSGSSSNIG | |
| DMSWVRQAPGKCLEWVSVIYPDDGNTYYADSV | | NNYVTWYQQLPGTAPKLLIYADSHRPSGVP | |
| KGRFTISRDNSKNTLYLQMNSLRAEDAAVYYC | | DRFSGSKSGTSASLAISGLRSEDEADYYCA | |
| AKHGGQKPTTKSSSAYGMDGWGQGTLVTVSS | | TWDYSLSGYVFGCGTKLTVL | |
| (SEQ ID NO: 65) | | (SEQ ID NO: 66) | |
| CDR Sequences (Kabat) | | CDR Sequences (Kabat) | |
| CDR-H1 | GYDMS | CDR-L1 | SGSSSNIGNNYVT |
| | (SEQ ID NO: 67) | | (SEQ ID NO: 70) |
| CDR-H2 | VIYPDDGNTYYADSVKG | CDR-L2 | ADSHRPS |
| | (SEQ ID NO: 68) | | (SEQ ID NO: 71) |
| CDR-H3 | HGGQKPTTKSSSAYGMDG | CDR-L3 | ATWDYSLSGYV |
| | (SEQ ID NO: 69) | | (SEQ ID NO: 72) |
| CDR Sequences (IMGT) | | CDR Sequences (IMGT) | |
| CDR-H1 | GFTFSGYD | CDR-L1 | SSNIGNNY |
| | (SEQ ID NO: 73) | | (SEQ ID NO: 76) |
| CDR-H2 | IYPDDGNT | CDR-L2 | AD |
| | (SEQ ID NO: 74) | CDR-L3 | ATWDYSLSGYV |
| CDR-H3 | AKHGGQKPTTKSSSAYGMDG | | (SEQ ID NO: 77) |
| | (SEQ ID NO: 75) | | |
| CDR Sequences (Chothia) | | CDR Sequences (Chothia) | |
| CDR-H1 | GFTFSGY | CDR-L1 | SGSSSNIGNNYVT |
| | (SEQ ID NO: 78) | | (SEQ ID NO: 81) |
| CDR-H2 | YPDDGN | CDR-L2 | ADSHRPS |
| | (SEQ ID NO: 79) | | (SEQ ID NO: 82) |
| CDR-H3 | HGGQKPTTKSSSAYGMDG | CDR-L3 | ATWDYSLSGYV |
| | (SEQ ID NO: 80) | | (SEQ ID NO: 83) |

TABLE 1E
4-1BB Antibody Sequences (clone ABLPNB.07)

| Heavy chain variable region | | Light chain variable region | |
|---|---|---|---|
| EVQLLESGGGLVQPGGSLRLSCAASGFTFSSY | | QSVLTQPPSASGTPGQRVTISCSGSSSNIG | |
| DMSWVRQAPGKCLEWVSWISYSGGSIYYADSV | | NNYVTWYQQLPGTAPKLLIYADSHRPSGVP | |
| KGRFTISRDNSKNTLYLQMNSLRAEDTAVYYC | | DRFSGSKSGTSASLAISGLRSEDEADYYCA | |
| ARDAQRNSMREFDYWGQGTLVTVSS | | TWDYSLSGYVFGCGTKLTVL | |
| (SEQ ID NO: 84) | | (SEQ ID NO: 85) | |
| CDR Sequences (Kabat) | | CDR Sequences (Kabat) | |
| CDR-H1 | SYDMS | CDR-L1 | SGSSSNIGNNYVT |
| | (SEQ ID NO: 86) | | (SEQ ID NO: 89) |
| CDR-H2 | WISYSGGSIYYADSVKG | CDR-L2 | ADSHRPS |
| | (SEQ ID NO: 87) | | (SEQ ID NO: 90) |
| CDR-H3 | DAQRNSMREFDY | CDR-L3 | ATWDYSLSGYV |
| | (SEQ ID NO: 88) | | (SEQ ID NO: 91) |
| CDR Sequences (IMGT) | | CDR Sequences (IMGT) | |
| CDR-H1 | GFTFSSYD | CDR-L1 | SSNIGNNY |
| | (SEQ ID NO: 92) | | (SEQ ID NO: 95) |
| CDR-H2 | ISYSGGSI | CDR-L2 | AD |
| | (SEQ ID NO: 93) | CDR-L3 | ATWDYSLSGYV |
| CDR-H3 | ARDAQRNSMREFDY | | (SEQ ID NO: 96) |
| | (SEQ ID NO: 94) | | |
| CDR Sequences (Chothia) | | CDR Sequences (Chothia) | |
| CDR-H1 | GFTFSSY | CDR-L1 | SGSSSNIGNNYVT |
| | (SEQ ID NO: 97) | | (SEQ ID NO: 100) |
| CDR-H2 | SYSGGS | CDR-L2 | ADSHRPS |
| | (SEQ ID NO: 98) | | (SEQ ID NO: 101) |
| CDR-H3 | DAQRNSMREFDY | CDR-L3 | ATWDYSLSGYV |
| | (SEQ ID NO: 99) | | (SEQ ID NO: 102) |

TABLE 1F
4-1BB Antibody Sequences (acasunlimab (4-1BB-binding arm))

| Heavy chain variable region | | Light chain variable region | |
|---|---|---|---|
| EVQLVESGGGLVQPGRSLRLSCTASGFSLNDY | | DIVMTQSPSSLSASVGDRVTITCQASEDIS | |
| WMSWVRQAPGKGLEWVGYIDVGGSLYYAASVK | | SYLAWYQQKPGKAPKRLIYGASDLASGVPS | |
| GRFTISRDDSKSIAYLQMNSLKTEDTAVYYCA | | RFSASGSGTDYTFTISSLQPEDIATYYCHY | |
| RGGLTYGFDLWGQGTLVTVSS | | YATISGLGVAFGGGTKVEIK | |
| (SEQ ID NO: 103) | | (SEQ ID NO: 104)ADG | |
| CDR Sequences (Kabat) | | CDR Sequences (Kabat) | |
| CDR-H1 | DYWMS | CDR-L1 | QASEDISSYLA |
| | (SEQ ID NO: 105) | | (SEQ ID NO: 108) |
| CDR-H2 | YIDVGGSLYYAASVKG | CDR-L2 | GASDLAS |
| | (SEQ ID NO: 106) | | (SEQ ID NO: 109) |
| CDR-H3 | GGLTYGFDL | CDR-L3 | HYYATISGLGVA |
| | (SEQ ID NO: 107) | | (SEQ ID NO: 110) |
| CDR Sequences (IMGT) | | CDR Sequences (IMGT) | |
| CDR-H1 | GFSLNDYW | CDR-L1 | EDISSY |
| | (SEQ ID NO: 111) | | (SEQ ID NO: 114) |
| CDR-H2 | IDVGGSL | CDR-L2 | GA |
| | (SEQ ID NO: 112) | CDR-L3 | HYYATISGLGVA |
| CDR-L3 | ARGGLTYGFDL | | (SEQ ID NO: 115) |
| | (SEQ ID NO: 113) | | |
| CDR Sequences (Chothia) | | CDR Sequences (Chothia) | |
| CDR-H1 | GFSLNDY | CDR-L1 | QASEDISSYLA |
| | (SEQ ID NO: 116) | | (SEQ ID NO: 119) |
| CDR-H2 | DVGGS | CDR-L2 | GASDLAS |
| | (SEQ ID NO: 117) | | (SEQ ID NO: 120) |
| CDR-H3 | GGLTYGFDL | CDR-L3 | HYYATISGLGVA |
| | (SEQ ID NO: 118) | | (SEQ ID NO: 121) |

TABLE 1G
4-1BB Antibody Sequences (clone AG10058)

| Heavy chain variable region | | Light chain variable region | |
|---|---|---|---|
| EVQLVESGGGLVQPGGSLRLSCAASGFSLSTS | | DIQLTQSPSSLSASVGDRVTITCRASQSVS | |
| GVGVGWIRQAPGKGLEWLALIDWDDDKYYSPS | | PYLAWYQQKPGKAPKLLIYDASSLESGVPS | |
| LKSRLTISRDNSKNTLYLQLNSLRAEDTAVYY | | RFSGSGSGTDFTLTISSLQPEDFATYYCQQ | |
| CARGGSDTVLGDWFAYWGQGTLVTVSS | | GYSLWTFGQGTKVEIK | |
| (SEQ ID NO: 122) | | (SEQ ID NO: 123) | |
| CDR Sequences (Kabat) | | CDR Sequences (Kabat) | |
| CDR-H1 | TSGVGVG | CDR-L1 | RASQSVSPYLA |
| | (SEQ ID NO: 124) | | (SEQ ID NO: 127) |
| CDR-H2 | LIDWDDDKYYSPSLKS | CDR-L2 | DASSLES |
| | (SEQ ID NO: 125) | | (SEQ ID NO: 128) |
| CDR-H3 | GGSDTVLGDWFAY | CDR-L3 | QQGYSLWT |
| | (SEQ ID NO: 126) | | (SEQ ID NO: 129) |
| CDR Sequences (IMGT) | | CDR Sequences (IMGT) | |
| CDR-H1 | GFSLSTSGVG | CDR-L1 | QSVSPY |
| | (SEQ ID NO: 130) | | (SEQ ID NO: 133) |
| CDR-H2 | IDWDDDK | CDR-L2 | DA |
| | (SEQ ID NO: 131) | CDR-L3 | QQGYSLWT |
| CDR-H3 | ARGGSDTVLGDWFAY | | (SEQ ID NO: 134) |
| | (SEQ ID NO: 132) | | |
| CDR Sequences (Chothia) | | CDR Sequences (Chothia) | |
| CDR-H1 | GFSLSTSGV | CDR-L1 | RASQSVSPYLA |
| | (SEQ ID NO: 135) | | (SEQ ID NO: 138) |
| CDR-H2 | DWDDD | CDR-L2 | DASSLES |
| | (SEQ ID NO: 136) | | (SEQ ID NO: 139) |
| CDR-H3 | GGSDTVLGDWFAY | CDR-L3 | QQGYSLWT |
| | (SEQ ID NO: 137) | | (SEQ ID NO: 140) |

TABLE 1H
4-1BB Antibody Sequences (clone AG10131)

| Heavy chain variable region | | Light chain variable region | |
|---|---|---|---|
| EVQLVESGGGLVQPGGSLRLSCAASGFSLSTG | | DIQLTQSPSSLSASVGDRVTITCRASQSIG | |
| GVGVGWIRQAPGKGLEWLALIDWADDKYYSPS | | SYLAWYQQKPGKAPKLLIYDASNLETGVPS | |
| LKSRLTISRDNSKNTLYLQLNSLRAEDTAVYY | | RFSGSGSGTDFTLTISSLQPEDFATYYCQQ | |
| CARGGSDTVIGDWFAYWGQGTLVTVSS (SEQ | | GYYLWTFGQGTKVEIK | |
| ID NO: 141) | | (SEQ ID NO: 142) | |
| CDR Sequences (Kabat) | | CDR Sequences (Kabat) | |
| CDR-H1 | TGGVGVG | CDR-L1 | RASQSIGSYLA |
| | (SEQ ID NO: 143) | | (SEQ ID NO: 146) |
| CDR-H2 | LIDWADDKYYSPSLKS | CDR-L2 | DASNLET |
| | (SEQ ID NO: 144) | | (SEQ ID NO: 147) |
| CDR-H3 | GGSDTVIGDWFAY | CDR-L3 | QQGYYLWT |
| | (SEQ ID NO: 145) | | (SEQ ID NO: 148) |
| CDR Sequences (IMGT) | | CDR Sequences (IMGT) | |
| CDR-H1 | GFSLSTGGVG | CDR-L1 | QSIGSY |
| | (SEQ ID NO: 149) | | (SEQ ID NO: 152) |
| CDR-H2 | IDWADDK | CDR-L2 | DA |
| | (SEQ ID NO: 150) | CDR-L3 | QQGYYLWT |
| CDR-H3 | ARGGSDTVIGDWFAY | | (SEQ ID NO: 153) |
| | (SEQ ID NO: 151) | | |
| CDR Sequences (Chothia) | | CDR Sequences (Chothia) | |
| CDR-H1 | GFSLSTGGV | CDR-L1 | RASQSIGSYLA |
| | (SEQ ID NO: 154) | | (SEQ ID NO: 157) |
| CDR-H2 | DWADD | CDR-L2 | DASNLET |
| | (SEQ ID NO: 155) | | (SEQ ID NO: 158) |
| CDR-H3 | GGSDTVIGDWFAY | CDR-L3 | QQGYYLWT |
| | (SEQ ID NO: 156) | | (SEQ ID NO: 159) |

TABLE 1I
4-1BB Antibody Sequences (clone BA001)

| Heavy chain variable region | | Light chain variable region | |
|---|---|---|---|
| QVQLVQSGAEVKKPGASVKVSCKASGYTFTGY | | SYVLTQPPSVSVAPGETARITCGGDDIGDK | |
| YMHWVRQAPGQGLEWMGWINPNSGGTNYAQKF | | RVHWYQKKPDQAPVLVVYEDRYRPSGIPER | |
| QGRVTMTRDTSISTAYMELSRLRSDDTAVYYC | | ISGSNSGNTATLTLSRVEAGDEADYYCQVW | |
| AREPGYYGSGLDYWGQGTLVTVSS (SEQ ID | | DSSSDHPGVEGGGTQLIIL (SEQ ID | |
| NO: 160) | | NO: 161) | |
| CDR Sequences (Kabat) | | CDR Sequences (Kabat) | |
| CDR-H1 | GYYMH | CDR-L1 | GGDDIGDKRVH |
| | (SEQ ID NO: 162) | | (SEQ ID NO: 165) |
| CDR-H2 | WINPNSGGTNYAQKFQG | CDR-L2 | EDRYRPS |
| | (SEQ ID NO: 163) | | (SEQ ID NO: 166) |
| CDR-H3 | EPGYYGSGLDY | CDR-L3 | QVWDSSSDHPGV |
| | (SEQ ID NO: 164) | | (SEQ ID NO: 167) |
| CDR Sequences (IMGT) | | CDR Sequences (IMGT) | |
| CDR-H1 | GYTFTGYY | CDR-L1 | DIGDKR |
| | (SEQ ID NO: 168) | | (SEQ ID NO: 171) |
| CDR-H2 | INPNSGGT | CDR-L2 | ED |
| | (SEQ ID NO: 169) | CDR-L3 | QVWDSSSDHPGV |
| CDR-H3 | AREPGYYGSGLDY | | (SEQ ID NO: 172) |
| | (SEQ ID NO: 170) | | |
| CDR Sequences (Chothia) | | CDR Sequences (Chothia) | |
| CDR-H1 | GYTFTGY | CDR-L1 | GGDDIGDKRVH |
| | (SEQ ID NO: 173) | | (SEQ ID NO: 176) |
| CDR-H2 | NPNSGG | CDR-L2 | EDRYRPS |
| | (SEQ ID NO: 174) | | (SEQ ID NO: 177) |
| CDR-H3 | EPGYYGSGLDY | CDR-L3 | QVWDSSSDHPGV |
| | (SEQ ID NO: 175) | | (SEQ ID NO: 178) |

TABLE 1J
4-1BB Antibody Sequences (clone 1630/1631)

| Heavy chain variable region | | Light chain variable region | |
|---|---|---|---|
| EVQLLESGGGLVQPGGSLRLSCAASGFTFGYS | | DIQMTQSPSSLSASVGDRVTITCRASQSIS | |
| YMSWVRQAPGKGLEWVSSIGSGSSYTYYADSV | | SYLNWYQQKPGKAPKLLIYAASSLQSGVPS | |
| KGRFTISRDNSKNTLYLQMNSLRAEDTAVYYC | | RFSGSGSGTDFTLTISSLQPEDFATYYCQQ | |
| ARVYSSPGIDYWGQGTLVTVSS | | YYTWVPFTFGQGTKLEIK | |
| (SEQ ID NO: 179) | | (SEQ ID NO: 180) | |
| CDR Sequences (Kabat) | | CDR Sequences (Kabat) | |
| CDR-H1 | YSYMS | CDR-L1 | RASQSISSYLN |
| | (SEQ ID NO: 181) | | (SEQ ID NO: 184) |
| CDR-H2 | SIGSGSSYTYYADSVKG | CDR-L2 | AASSLQS |
| | (SEQ ID NO: 182) | | (SEQ ID NO: 185) |
| CDR-H3 | VYSSPGIDY | CDR-L3 | QQYYTWVPFT |
| | (SEQ ID NO: 183) | | (SEQ ID NO: 186) |
| CDR Sequences (IMGT) | | CDR Sequences (IMGT) | |
| CDR-H1 | GFTFGYSY | CDR-L1 | QSISSY |
| | (SEQ ID NO: 187) | | (SEQ ID NO: 190) |
| CDR-H2 | IGSGSSYT | CDR-L2 | AA |
| | (SEQ ID NO: 188) | CDR-L3 | QQYYTWVPFT |
| CDR-H3 | ARVYSSPGIDY | | (SEQ ID NO: 191) |
| | (SEQ ID NO: 189) | | |
| CDR Sequences (Chothia) | | CDR Sequences (Chothia) | |
| CDR-H1 | GFTFGYS | CDR-L1 | RASQSISSYLN |
| | (SEQ ID NO: 192) | | (SEQ ID NO: 195) |
| CDR-H2 | GSGSSY | CDR-L2 | AASSLQS |
| | (SEQ ID NO: 193) | | (SEQ ID NO: 196) |
| CDR-H3 | VYSSPGIDY | CDR-L3 | QQYYTWVPFT |
| | (SEQ ID NO: 194) | | (SEQ ID NO: 197) |

TABLE 1K
4-1BB Antibody Sequences (clone 2674/2675)

| Heavy chain variable region | | Light chain variable region | |
|---|---|---|---|
| EVQLLESGGGLVQPGGSLRLSCAASGFNFGYS | | DIQMTQSPSSLSASVGDRVTITCRASQSIG | |
| YMSWVRQAPGKGLEWVSSIGSTSSHTYYADSV | | STLNWYQQKPGKAPKLLIYGASSLQSGVPS | |
| KGRFTISRDNSKNTLYLQMNSLRAEDTAVYYC | | RFSGSGSGTDFTLTISSLQPEDFATYYCQQ | |
| ARVYSSPGIDYWGQGTLVTVSS | | YYTWVPFTFGQGTKLEIK | |
| (SEQ ID NO: 198) | | (SEQ ID NO: 199) | |
| CDR Sequences (Kabat) | | CDR Sequences (Kabat) | |
| CDR-H1 | YSYMS | CDR-L1 | RASQSIGSTLN |
| | (SEQ ID NO: 200) | | (SEQ ID NO: 203) |
| CDR-H2 | SIGSTSSHTYYADSVKG | CDR-L2 | GASSLQS |
| | (SEQ ID NO: 201) | | (SEQ ID NO: 204) |
| CDR-H3 | VYSSPGIDY | CDR-L3 | QQYYTWVPFT |
| | (SEQ ID NO: 202) | | (SEQ ID NO: 205) |
| CDR Sequences (IMGT) | | CDR Sequences (IMGT) | |
| CDR-H1 | GFNFGYSY | CDR-L1 | QSIGST |
| | (SEQ ID NO: 206) | | (SEQ ID NO: 209) |
| CDR-H2 | IGSTSSHT | CDR-L2 | GA |
| | (SEQ ID NO: 207) | CDR-L3 | QQYYTWVPFT |
| CDR-H3 | ARVYSSPGIDY | | (SEQ ID NO: 210) |
| | (SEQ ID NO: 208) | | |
| CDR Sequences (Chothia) | | CDR Sequences (Chothia) | |
| CDR-H1 | GFNFGYS | CDR-L1 | RASQSIGSTLN |
| | (SEQ ID NO: 211) | | (SEQ ID NO: 214) |
| CDR-H2 | GSTSSH | CDR-L2 | GASSLQS |
| | (SEQ ID NO: 212) | | (SEQ ID NO: 215) |
| CDR-H3 | VYSSPGIDY | CDR-L3 | QQYYTWVPFT |
| | (SEQ ID NO: 213) | | (SEQ ID NO: 216) |

TABLE 1L
4-1BB Antibody Sequences (clone mAb1)

| Heavy chain variable region | | Light chain variable region | |
|---|---|---|---|
| EVQLLESGGGLVQPGGSLRLSCAASGFTFSSY | | DIQMTQSPSSVSASVGDRVTITCRASQGIS | |
| AMSWVRQAPGKGLEWVSAISGSGGSTYYADSV | | SWLAWYQQKPGKAPKLLIYAASSLQSGVPS | |
| KGRFTISRDNSKNTLYLQMNSLRAEDTAVYYC | | RFSGSGSGTDFTLTISSLQPEDFATYYCQQ | |
| AKDSPFLLDDYYYYYYMDVWGKGTTVTVSS | | GHLFPITFGGGTKVEIK | |
| (SEQ ID NO: 217) | | (SEQ ID NO: 218) | |
| CDR Sequences (Kabat) | | CDR Sequences (Kabat) | |
| CDR-H1 | SYAMS | CDR-L1 | RASQGISSWLA |
| | (SEQ ID NO: 219) | | (SEQ ID NO: 222) |
| CDR-H2 | AISGSGGSTYYADSVKG | CDR-L2 | AASSLQS |
| | (SEQ ID NO: 220) | | (SEQ ID NO: 223) |
| CDR-H3 | DSPFLLDDYYYYYYMDV | CDR-L3 | QQGHLFPIT |
| | (SEQ ID NO: 221) | | (SEQ ID NO: 224) |
| CDR Sequences (IMGT) | | CDR Sequences (IMGT) | |
| CDR-H1 | GFTFSSYA | CDR-L1 | QGISSW |
| | (SEQ ID NO: 225) | | (SEQ ID NO: 228) |
| CDR-H2 | ISGSGGST | CDR-L2 | AA |
| | (SEQ ID NO: 226) | CDR-L3 | QQGHLFPIT |
| CDR-H3 | AKDSPFLLDDYYYYYYMDV | | (SEQ ID NO: 229) |
| | (SEQ ID NO: 227) | | |
| CDR Sequences (Chothia) | | CDR Sequences (Chothia) | |
| CDR-H1 | GFTFSSY | CDR-L1 | RASQGISSWLA |
| | (SEQ ID NO: 230) | | (SEQ ID NO: 233) |
| CDR-H2 | SGSGGS | CDR-L2 | AASSLQS |
| | (SEQ ID NO: 231) | | (SEQ ID NO: 234) |
| CDR-H3 | DSPFLLDDYYYYYYMDV | CDR-L3 | QQGHLFPIT |
| | (SEQ ID NO: 232) | | (SEQ ID NO: 235) |

TABLE 1M
4-1BB Antibody Sequences (clone mAb10)

| Heavy chain variable region | | Light chain variable region | |
|---|---|---|---|
| EVQLLESGGGLVQPGGSLRLSCAASGFTFYGY | | DIQMTQSPSSVSASVGDRVTITCRASQGIS | |
| AMSWVRQAPGKGLEWVAAISGSGDSTYYADSV | | SWLAWYQQKPGKAPKLLIYAASSLQSGVPS | |
| KGRFTISRDNSKNTLYLQMNSLRAEDTAVYYC | | RFSGSGSGTDFTLTISSLQPEDFATYYCQQ | |
| AKDSPFLLDDYYYYYYMDVWGKGTTVTVSS | | GHLFPITFGGGTKVEIK | |
| (SEQ ID NO: 236) | | (SEQ ID NO: 237) | |
| CDR Sequences (Kabat) | | CDR Sequences (Kabat) | |
| CDR-H1 | GYAMS | CDR-L1 | RASQGISSWLA |
| | (SEQ ID NO: 238) | | (SEQ ID NO: 241) |
| CDR-H2 | AISGSGDSTYYADSVKG | CDR-L2 | AASSLQS |
| | (SEQ ID NO: 239) | | (SEQ ID NO: 242) |
| CDR-H3 | DSPFLLDDYYYYYYMDV | CDR-L3 | QQGHLFPIT |
| | (SEQ ID NO: 240) | | (SEQ ID NO: 243) |
| CDR Sequences (IMGT) | | CDR Sequences (IMGT) | |
| CDR-H1 | GFTFYGYA | CDR-L1 | QGISSW |
| | (SEQ ID NO: 244) | | (SEQ ID NO: 247) |
| CDR-H2 | ISGSGDST | CDR-L2 | AA |
| | (SEQ ID NO: 245) | CDR-L3 | QQGHLFPIT |
| CDR-H3 | AKDSPFLLDDYYYYYYMDV | | (SEQ ID NO: 248) |
| | (SEQ ID NO: 246) | | |
| CDR Sequences (Chothia) | | CDR Sequences (Chothia) | |
| CDR-H1 | GFTFYGY | CDR-L1 | RASQGISSWLA |
| | (SEQ ID NO: 249) | | (SEQ ID NO: 252) |
| CDR-H2 | SGSGDS | CDR-L2 | AASSLQS |
| | (SEQ ID NO: 250) | | (SEQ ID NO: 253) |
| CDR-H3 | DSPFLLDDYYYYYYMDV | CDR-L3 | QQGHLFPIT |
| | (SEQ ID NO: 251) | | (SEQ ID NO: 254) |

TABLE 1N
4-1BB Antibody Sequences (clone mAb8)

| Heavy chain variable region | | Light chain variable region | |
|---|---|---|---|
| EVQLLESGGGLVQPGGSLRLSCAASGFTFRNY | | DIQMTQSPSSVSASVGDRVTITCRASQGIS | |
| AMSWVRQAPGKGLEWVSAISGSGDTTYYADSV | | SWLAWYQQKPGKAPKLLIYAASSLQSGVPS | |
| KGRFTISRDNSKNTLYLQMNSLRAEDTAVYYC | | RFSGSGSGTDFTLTISSLQPEDFATYYCQQ | |
| AKDSPFLLDDYYYYYYMDVWGKGTTVTVSS | | GHLFPITFGGGTKVEIK | |
| (SEQ ID NO: 255) | | (SEQ ID NO: 256) | |
| CDR Sequences (Kabat) | | CDR Sequences (Kabat) | |
| CDR-H1 | NYAMS | CDR-L1 | RASQGISSWLA |
| | (SEQ ID NO: 257) | | (SEQ ID NO: 260) |
| CDR-H2 | AISGSGDTTYYADSVKG | CDR-L2 | AASSLQS |
| | (SEQ ID NO: 258) | | (SEQ ID NO: 261) |
| CDR-H3 | DSPFLLDDYYYYYYMDV | CDR-L3 | QQGHLFPIT |
| | (SEQ ID NO: 259) | | (SEQ ID NO: 262) |
| CDR Sequences (IMGT) | | CDR Sequences (IMGT) | |
| CDR-H1 | GFTFRNYA | CDR-L1 | QGISSW |
| | (SEQ ID NO: 263) | | (SEQ ID NO: 266) |
| CDR-H2 | ISGSGDTT | CDR-L2 | AA |
| | (SEQ ID NO: 264) | CDR-L3 | QQGHLFPIT |
| CDR-H3 | AKDSPFLLDDYYYYYYMDV | | (SEQ ID NO: 267) |
| | (SEQ ID NO: 265) | | |
| CDR Sequences (Chothia) | | CDR Sequences (Chothia) | |
| CDR-H1 | GFTFRNY | CDR-L1 | RASQGISSWLA |
| | (SEQ ID NO: 268) | | (SEQ ID NO: 271) |
| CDR-H2 | SGSGDT | CDR-L2 | AASSLQS |
| | (SEQ ID NO: 269) | | (SEQ ID NO: 272) |
| CDR-H3 | DSPFLLDDYYYYYYMDV | CDR-L3 | QQGHLFPIT |
| | (SEQ ID NO: 270) | | (SEQ ID NO: 273) |

TABLE 1O
4-1BB Antibody Sequences (clone 3712)

| HCVR | | LCVR | |
|---|---|---|---|
| QVQLVQSGAEVKKPGASVKVSCKASGYTFAGF | | DIQMTQSPSSLSASVGDRVTITCRASQDIR | |
| EMHWVRQAPGQGLEWMGAIDPKTGGTDYNQKF | | SNLNWYQQKPGGAVKLLIYYTSRLHSGVPS | |
| KDRVTMTRDTSISTAYMELSRLRSDDTAVYYC | | RFSGSGSGTDYTLTISSLQPEDFATYFCQQ | |
| ARDLGYFDVWGQGTLVTVSS | | SEKLPR | |
| (SEQ ID NO: 274) | | (SEQ ID NO: 275) | |
| CDR Sequences (Kabat) | | CDR Sequences (Kabat) | |
| CDR-H1 | GFEMH | CDR-L1 | RASQDIRSNLN |
| | (SEQ ID NO: 276) | | (SEQ ID NO: 279) |
| CDR-H2 | AIDPKTGGTDYNQKFKD | CDR-L2 | YTSRLHS |
| | (SEQ ID NO: 277) | | (SEQ ID NO: 280) |
| CDR-H3 | DLGYFDV | CDR-L3 | QQSEKLPR |
| | (SEQ ID NO: 278) | | (SEQ ID NO: 281) |
| CDR Sequences (IMGT) | | CDR Sequences (IMGT) | |
| CDR-H1 | GYTFAGFE | CDR-L1 | QDIRSN |
| | (SEQ ID NO: 282) | | (SEQ ID NO: 285) |
| CDR-H2 | IDPKTGGT | CDR-L2 | YT |
| | (SEQ ID NO: 283) | CDR-L3 | QQSEKLPR |
| CDR-H3 | ARDLGYFDV | | (SEQ ID NO: 286) |
| | (SEQ ID NO: 284) | | |
| CDR Sequences (Chothia) | | CDR Sequences (Chothia) | |
| CDR-H1 | GYTFAGF | CDR-L1 | RASQDIRSNLN |
| | (SEQ ID NO: 287) | | (SEQ ID NO: 290) |
| CDR-H2 | DPKTGG | CDR-L2 | YTSRLHS |
| | (SEQ ID NO: 288) | | (SEQ ID NO: 291) |
| CDR-H3 | DLGYFDV | CDR-L3 | QQSEKLPR |
| | (SEQ ID NO: 289) | | (SEQ ID NO: 292) |

TABLE 1P
4-1BB Antibody Sequences (urelumab)

| Heavy chain variable region | | Light chain variable region | |
|---|---|---|---|
| QVQLQQWGAGLLKPSETLSLTCAVYGGSFSGY | | EIVLTQSPATLSLSPGERATLSCRASQSVS | |
| YWSWIRQSPEKGLEWIGEINHGGYVTYNPSLE | | SYLAWYQQKPGQAPRLLIYDASNRATGIPA | |
| SRVTISVDTSKNQFSLKLSSVTAADTAVYYCA | | RFSGSGSGTDFTLTISSLEPEDFAVYYCQQ | |
| RDYGPGNYDWYFDLWGRGTLVTVSS | | RSNWPPALTFGGGTKVEIK | |
| (SEQ ID NO: 293) | | (SEQ ID NO: 294) | |
| CDR Sequences (Kabat) | | CDR Sequences (Kabat) | |
| CDR-H1 | GYYWS | CDR-L1 | RASQSVSSYLA |
| | (SEQ ID NO: 295) | | (SEQ ID NO: 298) |
| CDR-H2 | EINHGGYVTYNPSLES | CDR-L2 | DASNRAT |
| | (SEQ ID NO: 296) | | (SEQ ID NO: 299) |
| CDR-H3 | DYGPGNYDWYFDL | CDR-L3 | QQRSNWPPALT |
| | (SEQ ID NO: 297) | | (SEQ ID NO: 300) |
| CDR Sequences (IMGT) | | CDR Sequences (IMGT) | |
| CDR-H1 | GGSFSGYY | CDR-L1 | QSVSSY |
| | (SEQ ID NO: 301) | | (SEQ ID NO: 304) |
| CDR-H2 | INHGGYV | CDR-L2 | DA |
| | (SEQ ID NO: 302) | CDR-L3 | QQRSNWPPALT |
| CDR-H3 | ARDYGPGNYDWYFDL | | (SEQ ID NO: 305) |
| | (SEQ ID NO: 303) | | |
| CDR Sequences (Chothia) | | CDR Sequences (Chothia) | |
| CDR-H1 | GGSFSGY | CDR-L1 | RASQSVSSYLA |
| | (SEQ ID NO: 306) | | (SEQ ID NO: 309) |
| CDR-H2 | NHGGY | CDR-L2 | DASNRAT |
| | (SEQ ID NO: 307) | | (SEQ ID NO: 310) |
| CDR-H3 | DYGPGNYDWYFDL | CDR-L3 | QQRSNWPPALT |
| | (SEQ ID NO: 308) | | (SEQ ID NO: 311) |

TABLE 1Q
4-1BB Antibody Sequences (Utomilumab)

| Heavy chain variable region | | Light chain variable region | |
|---|---|---|---|
| EVQLVQSGAEVKKPGESLRISCKGSGYSFSTY | | SYELTQPPSVSVSPGQTASITCSGDNIGDQ | |
| WISWVRQMPGKGLEWMGKIYPGDSYTNYSPSF | | YAHWYQQKPGQSPVLVIYQDKNRPSGIPER | |
| QGQVTISADKSISTAYLQWSSLKASDTAMYYC | | FSGSNSGNTATLTISGTQAMDEADYYCATY | |
| ARGYGIFDYWGQGTLVTVSS | | TGFGSLAVFGGGTKLTVL | |
| (SEQ ID NO: 312) | | (SEQ ID NO: 313) | |
| CDR Sequences (Kabat) | | CDR Sequences (Kabat) | |
| CDR-H1 | TYWIS | CDR-L1 | SGDNIGDQYAH |
| | (SEQ ID NO: 314) | | (SEQ ID NO: 317) |
| CDR-H2 | KIYPGDSYTNYSPSFQG | CDR-L2 | QDKNRPS |
| | (SEQ ID NO: 315) | | (SEQ ID NO: 318) |
| CDR-H3 | GYGIFDY | CDR-L3 | ATYTGFGSLAV |
| | (SEQ ID NO: 316) | | (SEQ ID NO: 319) |
| CDR Sequences (IMGT) | | CDR Sequences (IMGT) | |
| CDR-H1 | GYSFSTYW | CDR-L1 | NIGDQY |
| | (SEQ ID NO: 320) | | (SEQ ID NO: 323) |
| CDR-H2 | IYPGDSYT | CDR-L2 | QD |
| | (SEQ ID NO: 321) | | |
| CDR-H3 | ARGYGIFDY | CDR-L3 | ATYTGFGSLAV |
| | (SEQ ID NO: 322) | | (SEQ ID NO: 324) |
| CDR Sequences (Chothia) | | CDR Sequences (Chothia) | |
| CDR-H1 | GYSFSTY | CDR-L1 | SGDNIGDQYAH |
| | (SEQ ID NO: 325) | | (SEQ ID NO: 328) |
| CDR-H2 | YPGDSY | CDR-L2 | QDKNRPS |
| | (SEQ ID NO: 326) | | (SEQ ID NO: 329) |
| CDR-H3 | GYGIFDY | CDR-L3 | ATYTGFGSLAV |
| | (SEQ ID NO: 327) | | (SEQ ID NO: 330) |

TABLE 2A
OX40 Antibody Sequences (BMS-986178)

| Heavy chain variable region | | Light chain variable region | |
|---|---|---|---|
| EVQLVESGGGLVQPGGSLRLSCAASGFTFSSY | | DIQMTQSPSSLSASVGDRVTITCRASQGIS | |
| SMNWVRQAPGKGLEWVSYISSSSSTIDYADSV | | SWLAWYQQKPEKAPKSLIYAASSLQSGVPS | |
| KGRFTISRDNAKNSLYLQMNSLRDEDTAVYYC | | RFSGSGSGTDFTLTISSLQPEDFATYYCQQ | |
| ARESGWYLFDYWGQGTLVTVSS | | YNSYPPTFGGGTKVEIK | |
| (SEQ ID NO: 331) | | (SEQ ID NO: 332) | |
| CDR Sequences (Kabat) | | CDR Sequences (Kabat) | |
| CDR-H1 | SYSMN | CDR-L1 | RASQGISSWLA |
| | (SEQ ID NO: 333) | | (SEQ ID NO: 336) |
| CDR-H2 | YISSSSSTIDYADSVKG | CDR-L2 | AASSLQS |
| | (SEQ ID NO: 334) | | (SEQ ID NO: 337) |
| CDR-H3 | ESGWYLFDY | CDR-L3 | QQYNSYPPT |
| | (SEQ ID NO: 335) | | (SEQ ID NO: 338) |
| CDR Sequences (IMGT) | | CDR Sequences (IMGT) | |
| CDR-H1 | GFTFSSYS | CDR-L1 | QGISSW |
| | (SEQ ID NO: 339) | | (SEQ ID NO: 342) |
| CDR-H2 | ISSSSSTI | CDR-L2 | AA |
| | (SEQ ID NO: 340) | | |
| CDR-H3 | ARESGWYLFDY | CDR-L3 | QQYNSYPPT |
| | (SEQ ID NO: 341) | | (SEQ ID NO: 343) |
| CDR Sequences (Chothia) | | CDR Sequences (Chothia) | |
| CDR-H1 | GFTFSSY | CDR-L1 | RASQGISSWLA |
| | (SEQ ID NO: 344) | | (SEQ ID NO: 347) |
| CDR-H2 | SSSSST | CDR-L2 | AASSLQS |
| | (SEQ ID NO: 345) | | (SEQ ID NO: 348) |
| CDR-H3 | ESGWYLFDY | CDR-L3 | QQYNSYPPT |
| | (SEQ ID NO: 346) | | (SEQ ID NO: 349) |

TABLE 2B
OX40 Antibody Sequences (Cudarolimab (IBI101))

| Heavy chain variable region | | Light chain variable region | |
|---|---|---|---|
| QVQLVESGGGVVQPGRSLRLSCAASGFTFSSY | | DIQMTQSPSSLSASVGDRVTITCQASQDIS | |
| GMHWVRQAPGKGLEWVAVISYDGSNKYYADSV | | NYLNWYQQKPGKAPKLLIYDASNLETGVPS | |
| KGRFTISRDNSKNTLYLQMNSLRAEDTAVYYC | | RFSGSGSGTDFTFTISSLQPEDIATYYCQQ | |
| ARGRPWYSETGTSAFDIWGQGTMVTVSS | | SDHYPTFGGGTKVEIKR | |
| (SEQ ID NO: 350) | | (SEQ ID NO: 351) | |
| CDR Sequences (Kabat) | | CDR Sequences (Kabat) | |
| CDR-H1 | SYGMH | CDR-L1 | QASQDISNYLN |
| | (SEQ ID NO: 352) | | (SEQ ID NO: 355) |
| CDR-H2 | VISYDGSNKYYADSVKG | CDR-L2 | DASNLET |
| | (SEQ ID NO: 353) | | (SEQ ID NO: 356) |
| CDR-H3 | GRPWYSETGTSAFDI | CDR-L3 | QQSDHYPT |
| | (SEQ ID NO: 354) | | (SEQ ID NO: 357) |
| CDR Sequences (IMGT) | | CDR Sequences (IMGT) | |
| CDR-H1 | GFTFSSYG | CDR-L1 | QDISNY |
| | (SEQ ID NO: 358) | | (SEQ ID NO: 361) |
| CDR-H2 | ISYDGSNK | CDR-L2 | DA |
| | (SEQ ID NO: 359) | | |
| CDR-H3 | ARGRPWYSETGTSAFDI | CDR-L3 | QQSDHYPT |
| | (SEQ ID NO: 360) | | (SEQ ID NO: 362) |
| CDR Sequences (Chothia) | | CDR Sequences (Chothia) | |
| CDR-H1 | GFTFSSY | CDR-L1 | QASQDISNYLN |
| | (SEQ ID NO: 363) | | (SEQ ID NO: 366) |
| CDR-H2 | SYDGSN | CDR-L2 | DASNLET |
| | (SEQ ID NO: 364) | | (SEQ ID NO: 367) |
| CDR-H3 | GRPWYSETGTSAFDI | CDR-L3 | QQSDHYPT |
| | (SEQ ID NO: 365) | | (SEQ ID NO: 368) |

TABLE 2C
OX40 Antibody Sequences (Hu106-222)

| Heavy chain variable region | | Light chain variable region | |
|---|---|---|---|
| QVQLVQSGSELKKPGASVKVSCKASGYTFTDY | | DIQMTQSPSSLSASVGDRVTITCKASQDVS | |
| SMHWVRQAPGQGLKWMGWINTETGEPTYADDF | | TAVAWYQQKPGKAPKLLIYSASYLYTGVPS | |
| KGRFVFSLDTSVSTAYLQISSLKAEDTAVYYC | | RFSGSGSGTDFTFTISSLQPEDIATYYCQQ | |
| ANPYYDYVSYYAMDYWGQGTLVTVSS | | HYSTPRTFGQGTKLEIK | |
| (SEQ ID NO: 369) | | (SEQ ID NO: 370) | |
| CDR Sequences (Kabat) | | CDR Sequences (Kabat) | |
| CDR-H1 | DYSMH | CDR-L1 | KASQDVSTAVA |
| | (SEQ ID NO: 371) | | (SEQ ID NO: 374) |
| CDR-H2 | WINTETGEPTYADDFKG | CDR-L2 | SASYLYT |
| | (SEQ ID NO: 372) | | (SEQ ID NO: 375) |
| CDR-H3 | PYYDYVSYYAMDY | CDR-L3 | QQHYSTPRT |
| | (SEQ ID NO: 373) | | (SEQ ID NO: 376) |
| CDR Sequences (IMGT) | | CDR Sequences (IMGT) | |
| CDR-H1 | GYTFTDYS | CDR-L1 | QDVSTA |
| | (SEQ ID NO: 377) | | (SEQ ID NO: 380) |
| CDR-H2 | INTETGEP | CDR-L2 | SA |
| | (SEQ ID NO: 378) | | |
| CDR-H3 | ANPYYDYVSYYAMDY | CDR-L3 | QQHYSTPRT |
| | (SEQ ID NO: 379) | | (SEQ ID NO: 381) |
| CDR Sequences (Chothia) | | CDR Sequences (Chothia) | |
| CDR-H1 | GYTFTDY | CDR-L1 | KASQDVSTAVA |
| | (SEQ ID NO: 382) | | (SEQ ID NO: 385) |
| CDR-H2 | NTETGE | CDR-L2 | SASYLYT |
| | (SEQ ID NO: 383) | | (SEQ ID NO: 386) |
| CDR-H3 | PYYDYVSYYAMDY | CDR-L3 | QQHYSTPRT |
| | (SEQ ID NO: 384) | | (SEQ ID NO: 387) |

TABLE 2D
OX40 Antibody Sequences (Hu119-122)

| Heavy chain variable region | | Light chain variable region | |
|---|---|---|---|
| EVQLVESGGGLVQPGGSLRLSCAASEYEFPSH | | EIVLTQSPATLSLSPGERATLSCRASKSVS | |
| DMSWVRQAPGKGLELVAAINSDGGSTYYPDTM | | TSGYSYMHWYQQKPGQAPRLLIYLASNLES | |
| ERRFTISRDNAKNSLYLQMNSLRAEDTAVYYC | | GVPARFSGSGSGTDFTLTISSLEPEDFAVY | |
| ARHYDDYYAWFAYWGQGTLVTVSS | | YCQHSRELPLTFGGGTKVEIK | |
| (SEQ ID NO: 388) | | (SEQ ID NO: 389) | |
| CDR Sequences (Kabat) | | CDR Sequences (Kabat) | |
| CDR-H1 | SHDMS | CDR-L1 | RASKSVSTSGYSYMH |
| | (SEQ ID NO: 390) | | (SEQ ID NO: 393) |
| CDR-H2 | AINSDGGSTYYPDTMER | CDR-L2 | LASNLES |
| | (SEQ ID NO: 391) | | (SEQ ID NO: 394) |
| CDR-H3 | HYDDYYAWFAY | CDR-L3 | QHSRELPLT |
| | (SEQ ID NO: 392) | | (SEQ ID NO: 395) |
| CDR Sequences (IMGT) | | CDR Sequences (IMGT) | |
| CDR-H1 | EYEFPSHD | CDR-L1 | KSVSTSGYSY |
| | (SEQ ID NO: 396) | | (SEQ ID NO: 399) |
| CDR-H2 | INSDGGST | CDR-L2 | LA |
| | (SEQ ID NO: 397) | | |
| CDR-H3 | ARHYDDYYAWFAY | CDR-L3 | QHSRELPLT |
| | (SEQ ID NO: 398) | | (SEQ ID NO: 400) |
| CDR Sequences (Chothia) | | CDR Sequences (Chothia) | |
| CDR-H1 | EYEFPSH | CDR-L1 | RASKSVSTSGYSYMH |
| | (SEQ ID NO: 401) | | (SEQ ID NO: 404) |
| CDR-H2 | NSDGGS | CDR-L2 | LASNLES |
| | (SEQ ID NO: 402) | | (SEQ ID NO: 405) |
| CDR-H3 | HYDDYYAWFAY | CDR-L3 | QHSRELPLT |
| | (SEQ ID NO: 403) | | (SEQ ID NO: 406) |

TABLE 2E
OX40 Antibody Sequences (INCAGN1949)

| Heavy chain variable region | | Light chain variable region | |
|---|---|---|---|
| EVQLVESGGGLVQPGGSLKLSCAASGFTFSGS | | DIVMTQSPLSLPVTPGEPASISCRSSQSLL | |
| AMHWVRQASGKGLEWVGRIRSKANSYATAYAA | | HSNGYNYLDWYLQKPGQSPQLLIYLGSNRA | |
| SVKGRFTISRDDSKNTAYLQMNSLKTEDTAVY | | SGVPDRFSGSGSGTDFTLKISRVEAEDVGV | |
| YCTSGIYDSSGYDYWGQGTLVTVSS | | YYCMQALQTPLTFGGGTKVEIK | |
| (SEQ ID NO: 407) | | (SEQ ID NO: 408) | |
| CDR Sequences (Kabat) | | CDR Sequences (Kabat) | |
| CDR-H1 | GSAMH | CDR-L1 | RSSQSLLHSNGYNYLD |
| | (SEQ ID NO: 409) | | (SEQ ID NO: 412) |
| CDR-H2 | RIRSKANSYATAYAASVKG | CDR-L2 | LGSNRAS |
| | (SEQ ID NO: 410) | | (SEQ ID NO: 413) |
| CDR-H3 | GIYDSSGYDY | CDR-L3 | MQALQTPLT |
| | (SEQ ID NO: 411) | | (SEQ ID NO: 414) |
| CDR Sequences (IMGT) | | CDR Sequences (IMGT) | |
| CDR-H1 | GFTFSGSA | CDR-L1 | QSLLHSNGYNY |
| | (SEQ ID NO: 415) | | (SEQ ID NO: 418) |
| CDR-H2 | IRSKANSYAT | CDR-L2 | LG |
| | (SEQ ID NO: 416) | | |
| CDR-H3 | TSGIYDSSGYDY | CDR-L3 | MQALQTPLT |
| | (SEQ ID NO: 417) | | (SEQ ID NO: 419) |
| CDR Sequences (Chothia) | | CDR Sequences (Chothia) | |
| CDR-H1 | GFTFSGS | CDR-L1 | RSSQSLLHSNGYNYLD |
| | (SEQ ID NO: 420) | | (SEQ ID NO: 423) |
| CDR-H2 | RSKANSYA | CDR-L2 | LGSNRAS |
| | (SEQ ID NO: 421) | | (SEQ ID NO: 424) |
| CDR-H3 | GIYDSSGYDY | CDR-L3 | MQALQTPLT |
| | (SEQ ID NO: 422) | | (SEQ ID NO: 425) |

TABLE 2F
OX40 Antibody Sequences (Ivuxolimab (PF-04518600))

| Heavy chain variable region | | Light chain variable region | |
|---|---|---|---|
| EVQLVESGGGLVQPGGSLRLSCAASGFTFSSY | | DIQMTQSPSSLSASVGDRVTITCRASQGIS | |
| SMNWVRQAPGKGLEWVSYISSSSSTIDYADSV | | SWLAWYQQKPEKAPKSLIYAASSLQSGVPS | |
| KGRFTISRDNAKNSLYLQMNSLRDEDTAVYYC | | RFSGSGSGTDFTLTISSLQPEDFATYYCQQ | |
| ARESGWYLFDYWGQGTLVTVSS | | YNSYPPTFGGGTKVEIKR | |
| (SEQ ID NO: 426) | | (SEQ ID NO: 427) | |
| CDR Sequences (Kabat) | | CDR Sequences (Kabat) | |
| CDR-H1 | SYSMN | CDR-L1 | RASQGISSWLA |
| | (SEQ ID NO: 428) | | (SEQ ID NO: 431) |
| CDR-H2 | YISSSSSTIDYADSVKG | CDR-L2 | AASSLQS |
| | (SEQ ID NO: 429) | | (SEQ ID NO: 432) |
| CDR-H3 | ESGWYLFDY | CDR-L3 | QQYNSYPPT |
| | (SEQ ID NO: 430) | | (SEQ ID NO: 433) |
| CDR Sequences (IMGT) | | CDR Sequences (IMGT) | |
| CDR-H1 | GFTFSSYS | CDR-L1 | QGISSW |
| | (SEQ ID NO: 434) | | (SEQ ID NO: 437) |
| CDR-H2 | ISSSSSTI | CDR-L2 | AA |
| | (SEQ ID NO: 435) | | |
| CDR-H3 | ARESGWYLFDY | CDR-L3 | QQYNSYPPT |
| | (SEQ ID NO: 436) | | (SEQ ID NO: 438) |
| CDR Sequences (Chothia) | | CDR Sequences (Chothia) | |
| CDR-H1 | GFTFSSY | CDR-L1 | RASQGISSWLA |
| | (SEQ ID NO: 439) | | (SEQ ID NO: 442) |
| CDR-H2 | SSSSST | CDR-L2 | AASSLQS |
| | (SEQ ID NO: 440) | | (SEQ ID NO: 443) |
| CDR-H3 | ESGWYLFDY | CDR-L3 | QQYNSYPPT |
| | (SEQ ID NO: 441) | | (SEQ ID NO: 444) |

TABLE 2G
OX40 Antibody Sequences (MEDI6469)

| Heavy chain variable region | | Light chain variable region | |
|---|---|---|---|
| EVQLQESGPSLVKPSQTLSLTCSVTGDSFTSG | | DIQMTQTTSSLSASLGDRVTISCRASQDIS | |
| YWNWIRKFPGNRLEYMGYISYNGITYHNPSLK | | NYLNWYQQKPDGTVKLLIYYTSKLHSGVPS | |
| SRISITRDTSKNHYYLQLNSVTTEDTATYFCA | | RFSGSGSRTDYSLTITDLDQEDIATYFCQQ | |
| RYRYDYDGGHAMDYWGQGTLVTVSS | | GSALPWTFGQGTKVEIK | |
| (SEQ ID NO: 445) | | (SEQ ID NO: 446) | |
| CDR Sequences (Kabat) | | CDR Sequences (Kabat) | |
| CDR-H1 | SGYWN | CDR-L1 | RASQDISNYLN |
| | (SEQ ID NO: 447) | | (SEQ ID NO: 450) |
| CDR-H2 | YISYNGITYHNPSLKS | CDR-L2 | YTSKLHS |
| | (SEQ ID NO: 448) | | (SEQ ID NO: 451) |
| CDR-H3 | YRYDYDGGHAMDY | CDR-L3 | QQGSALPWT |
| | (SEQ ID NO: 449) | | (SEQ ID NO: 452) |
| CDR Sequences (IMGT) | | CDR Sequences (IMGT) | |
| CDR-H1 | GDSFTSGY | CDR-L1 | QDISNY |
| | (SEQ ID NO: 453) | | (SEQ ID NO: 456) |
| CDR-H2 | ISYNGIT | CDR-L2 | YT |
| | (SEQ ID NO: 454) | | |
| CDR-H3 | ARYRYDYDGGHAMDY | CDR-L3 | QQGSALPWT |
| | (SEQ ID NO: 455) | | (SEQ ID NO: 457) |
| CDR Sequences (Chothia) | | CDR Sequences (Chothia) | |
| CDR-H1 | GDSFTSG | CDR-L1 | RASQDISNYLN |
| | (SEQ ID NO: 458) | | (SEQ ID NO: 461) |
| CDR-H2 | SYNGI | CDR-L2 | YTSKLHS |
| | (SEQ ID NO: 459) | | (SEQ ID NO: 462) |
| CDR-H3 | YRYDYDGGHAMDY | CDR-L3 | QQGSALPWT |
| | (SEQ ID NO: 460) | | (SEQ ID NO: 463) |

TABLE 2H
OX40 Antibody Sequences (MOXR0916 (RG7888))

| Heavy chain variable region | | Light chain variable region | |
|---|---|---|---|
| EVQLVQSGAEVKKPGASVKVSCKASGYTFTDS | | DIQMTQSPSSLSASVGDRVTITCRASQDIS | |
| YMSWVRQAPGQGLEWIGDMYPDNGDSSYNQKF | | NYLNWYQQKPGKAPKLLIYYTSRLRSGVPS | |
| RERVTITRDTSTSTAYLELSSLRSEDTAVYYC | | RFSGSGSGTDFTLTISSLQPEDFATYYCQQ | |
| VLAPRWYFSVWGQGTLVTVSS | | GHTLPPTFGQGTKVEIK | |
| (SEQ ID NO: 464) | | (SEQ ID NO: 465) | |
| CDR Sequences (Kabat) | | CDR Sequences (Kabat) | |
| CDR-H1 | DSYMS | CDR-L1 | RASQDISNYLN |
| | (SEQ ID NO: 466) | | (SEQ ID NO: 469) |
| CDR-H2 | DMYPDNGDSSYNQKFRE | CDR-L2 | YTSRLRS |
| | (SEQ ID NO: 467) | | (SEQ ID NO: 470) |
| CDR-H3 | APRWYFSV | CDR-L3 | QQGHTLPPT |
| | (SEQ ID NO: 468) | | (SEQ ID NO: 471) |
| CDR Sequences (IMGT) | | CDR Sequences (IMGT) | |
| CDR-H1 | GYTFTDSY | CDR-L1 | QDISNY |
| | (SEQ ID NO: 472) | | (SEQ ID NO: 475) |
| CDR-H2 | MYPDNGDS | CDR-L2 | YT |
| | (SEQ ID NO: 473) | | |
| CDR-H3 | VLAPRWYFSV | CDR-L3 | QQGHTLPPT |
| | (SEQ ID NO: 474) | | (SEQ ID NO: 476) |
| CDR Sequences (Chothia) | | CDR Sequences (Chothia) | |
| CDR-H1 | GYTFTDS | CDR-L1 | RASQDISNYLN |
| | (SEQ ID NO: 477) | | (SEQ ID NO: 480) |
| CDR-H2 | YPDNGD | CDR-L2 | YTSRLRS |
| | (SEQ ID NO: 478) | | (SEQ ID NO: 481) |
| CDR-H3 | APRWYFSV | CDR-L3 | QQGHTLPPT |
| | (SEQ ID NO: 479) | | (SEQ ID NO: 482) |

TABLE 2I
OX40 Antibody Sequences (Revdofilimab (ABBV368))

| Heavy chain variable region | | Light chain variable region | |
|---|---|---|---|
| EVQLVESGGGLVQPGGSLRLSCAASGFTFSRY | | DIVMTQSPDSLAVSLGERATINCKASQSVD | |
| GMSWVRQAPGKGLELVATINSNGGRTYYPDSV | | YDGDSYMHWYQQKPGQPPKLLIYAASILES | |
| KGRFTISRDNAKNSLYLQMNSLRAEDTAVYYC | | GVPDRFSGSGSGTDFTLTISSLQAEDVAVY | |
| AREGITTAYAMDYWGQGTTVTVSS | | YCQQSNEDPRTFGGGTKVEIKR | |
| (SEQ ID NO: 483) | | (SEQ ID NO: 484) | |
| CDR Sequences (Kabat) | | CDR Sequences (Kabat) | |
| CDR-H1 | RYGMS | CDR-L1 | KASQSVDYDGDSYMH |
| | (SEQ ID NO: 485) | | (SEQ ID NO: 488) |
| CDR-H2 | TINSNGGRTYYPDSVKG | CDR-L2 | AASILES |
| | (SEQ ID NO: 486) | | (SEQ ID NO: 489) |
| CDR-H3 | EGITTAYAMDY | CDR-L3 | QQSNEDPRT |
| | (SEQ ID NO: 487) | | (SEQ ID NO: 490) |
| CDR Sequences (IMGT) | | CDR Sequences (IMGT) | |
| CDR-H1 | GFTFSRYG | CDR-L1 | QSVDYDGDSY |
| | (SEQ ID NO: 491) | | (SEQ ID NO: 494) |
| CDR-H2 | INSNGGRT | CDR-L2 | AA |
| | (SEQ ID NO: 492) | | |
| CDR-H3 | AREGITTAYAMDY | CDR-L3 | QQSNEDPRT |
| | (SEQ ID NO: 493) | | (SEQ ID NO: 495) |
| CDR Sequences (Chothia) | | CDR Sequences (Chothia) | |
| CDR-H1 | GFTFSRY | CDR-L1 | KASQSVDYDGDSYMH |
| | (SEQ ID NO: 496) | | (SEQ ID NO: 499) |
| CDR-H2 | NSNGGR | CDR-L2 | AASILES |
| | (SEQ ID NO: 497) | | (SEQ ID NO: 500) |
| CDR-H3 | EGITTAYAMDY | CDR-L3 | QQSNEDPRT |
| | (SEQ ID NO: 498) | | (SEQ ID NO: 501) |

TABLE 2J
OX40 Antibody Sequences (Tavolimab)

| Heavy chain variable region | | Light chain variable region | |
|---|---|---|---|
| QVQLQESGPGLVKPSQTLSLTCAVYGGSFSSG | | DIQMTQSPSSLSASVGDRVTITCRASQDIS | |
| YWNWIRKHPGKGLEYIGYISYNGITYHNPSLK | | NYLNWYQQKPGKAPKLLIYYTSKLHSGVPS | |
| SRITINRDTSKNQYSLQLNSVTPEDTAVYYCA | | RFSGSGSGTDYTLTISSLQPEDFATYYCQQ | |
| RYKYDYDGGHAMDYWGQGTLVTVSS | | GSALPWTFGQGTKVEIKR | |
| (SEQ ID NO: 502) | | (SEQ ID NO: 503) | |
| CDR Sequences (Kabat) | | CDR Sequences (Kabat) | |
| CDR-H1 | SGYWN | CDR-L1 | RASQDISNYLN |
| | (SEQ ID NO: 504) | | (SEQ ID NO: 507) |
| CDR-H2 | YISYNGITYHNPSLKS | CDR-L2 | YTSKLHS |
| | (SEQ ID NO: 505) | | (SEQ ID NO: 508) |
| CDR-H3 | YKYDYDGGHAMDY | CDR-L3 | QQGSALPWT |
| | (SEQ ID NO: 506) | | (SEQ ID NO: 509) |
| CDR Sequences (IMGT) | | CDR Sequences (IMGT) | |
| CDR-H1 | GGSFSSGY | CDR-L1 | QDISNY |
| | (SEQ ID NO: 510) | | (SEQ ID NO: 513) |
| CDR-H2 | ISYNGIT | CDR-L2 | YT |
| | (SEQ ID NO: 511) | | |
| CDR-H3 | ARYKYDYDGGHAMDY | CDR-L3 | QQGSALPWT |
| | (SEQ ID NO: 512) | | (SEQ ID NO: 514) |
| CDR Sequences (Chothia) | | CDR Sequences (Chothia) | |
| CDR-H1 | GGSFSSG | CDR-L1 | RASQDISNYLN |
| | (SEQ ID NO: 515) | | (SEQ ID NO: 518) |
| CDR-H2 | SYNGI | CDR-L2 | YTSKLHS |
| | (SEQ ID NO: 516) | | (SEQ ID NO: 519) |
| CDR-H3 | YKYDYDGGHAMDY | CDR-L3 | QQGSALPWT |
| | (SEQ ID NO: 517) | | (SEQ ID NO: 520) |

TABLE 2K
OX40 Antibody Sequences (Vonlerolizumab (Pogalizumab))

| Heavy chain variable region | | Light chain variable region | |
|---|---|---|---|
| EVQLVQSGAEVKKPGASVKVSCKASGYTFTDS | | DIQMTQSPSSLSASVGDRVTITCRASQDIS | |
| YMSWVRQAPGQGLEWIGDMYPDNGDSSYNQKF | | NYLNWYQQKPGKAPKLLIYYTSRLRSGVPS | |
| RERVTITRDTSTSTAYLELSSLRSEDTAVYYC | | RFSGSGSGTDFTLTISSLQPEDFATYYCQQ | |
| VLAPRWYFSVWGQGTLVTVSS | | GHTLPPTFGQGTKVEIKR | |
| (SEQ ID NO: 521) | | (SEQ ID NO: 522) | |
| CDR Sequences (Kabat) | | CDR Sequences (Kabat) | |
| CDR-H1 | DSYMS | CDR-L1 | RASQDISNYLN |
| | (SEQ ID NO: 523) | | (SEQ ID NO: 526) |
| CDR-H2 | DMYPDNGDSSYNQKFRE | CDR-L2 | YTSRLRS |
| | (SEQ ID NO: 524) | | (SEQ ID NO: 527) |
| CDR-H3 | APRWYFSV | CDR-L3 | QQGHTLPPT |
| | (SEQ ID NO: 525) | | (SEQ ID NO: 528) |
| CDR Sequences (IMGT) | | CDR Sequences (IMGT) | |
| CDR-H1 | GYTFTDSY | CDR-L1 | QDISNY |
| | (SEQ ID NO: 529) | | (SEQ ID NO: 532) |
| CDR-H2 | MYPDNGDS | CDR-L2 | YT |
| | (SEQ ID NO: 530) | | |
| CDR-H3 | VLAPRWYFSV | CDR-L3 | QQGHTLPPT |
| | (SEQ ID NO: 531) | | (SEQ ID NO: 533) |
| CDR Sequences (Chothia) | | CDR Sequences (Chothia) | |
| CDR-H1 | GYTFTDS | CDR-L1 | RASQDISNYLN |
| | (SEQ ID NO: 534) | | (SEQ ID NO: 537) |
| CDR-H2 | YPDNGD | CDR-L2 | YTSRLRS |
| | (SEQ ID NO: 535) | | (SEQ ID NO: 538) |
| CDR-H3 | APRWYFSV | CDR-L3 | QQGHTLPPT |
| | (SEQ ID NO: 536) | | (SEQ ID NO: 539) |

### B. Tumor-Binding Moieties

Tumor-binding moieties are generally capable of binding (e.g., specifically binding) to a tumor-associated antigen (e.g., a tumor-specific antigen) expressed on a tumor cell or a cell that supports a tumor (e.g., a stromal cell.) In some embodiments, the tumor-binding moiety is capable of binding (e.g., specifically binding) to PD-L1, EGFR, or B7-H3.

In some embodiments, the tumor-binding moiety comprises an antibody fragment comprising a heavy chain variable region (V_{H}) and light chain variable region (V_{L} or V_{K}), wherein the CDRs of the V_{H} and the V_{L} or V_{K} (CDR-H1, CDR-H2, CDR-H3, CDR-L1, CDR-L2, and CDR-L3) have sequences which are similar to (e.g., each having at most one or two amino acids differing from) the CDRs of a PD-L1, EGFR, or B7-H3 antibody (e.g., a human or humanized PD-L1, EGFR, or B7-H3 antibody).

In some embodiments, the tumor-binding moiety comprises an antibody fragment comprising a heavy chain variable region (V_{H}) and light chain variable region (V_{L} or V_{K}), wherein the CDRs of the V_{H} and the V_{L} or V_{K} have sequences which are identical to those of the heavy and light chain CDRs of a PD-L1, EGFR, or B7-H3 antibody (e.g., a human or humanized PD-L1, EGFR, or B7-H3 antibody), except for one or two amino acid substitutions total across all six CDRs.

In some embodiments, the tumor-binding moiety comprises an antibody fragment comprising a heavy chain variable region (V_{H}) and light chain variable region (V_{L} or V_{K}), wherein the CDRs of the V_{H} and the V_{L} or V_{K} are identical to those of the heavy and light chain CDRs of a PD-L1, EGFR, or B7-H3 antibody (e.g., a human or humanized PD-L1, EGFR, or B7-H3 antibody).

In some embodiments, the tumor-binding moiety comprises an antibody fragment comprising heavy and light chain variable regions (e.g., V_{H} and V_{L} or V_{K}) with sequences that have at least 85%, at least 87.5%, at least 90%, at least 91%, at least 92%, at least 93%, at least 94%, at least 95%, at least 96%, at least 97%, at least 98% or at least 99% identity to those of the heavy and light chain variable regions of a PD-L1, EGFR, or B7-H3 antibody, e.g., a human or humanized PD-L1, EGFR, or B7-H3 antibody. In some embodiments, the tumor-binding moiety is an antibody fragment comprising heavy and light chain variable regions (e.g., V_{H} and V_{L} or V_{K}) with sequences identical to those of the heavy and light chain variable regions of a PD-L1, EGFR, or B7-H3 antibody (e.g., a human or humanized PD-L1, EGFR, or B7-H3 antibody).

Non-limiting examples of PD-L1 antibodies include atezolizumab, avelumab, and durvalumab, whose heavy chain variable region, light chain variable region, and CDR sequences are depicted in Tables 3A-3C.

TABLE 3A
PD-L1 Antibody Sequences (Atezolizumab)

| Heavy chain variable region | | Light chain variable region | |
|---|---|---|---|
| EVQLVESGGGLVQPGGSLRLSCAASGFTFS | | DIQMTQSPSSLSASVGDRVTITCRASQD | |
| DSWIHWVRQAPGKGLEWVAWISPYGGSTYY | | VSTAVAWYQQKPGKAPKLLIYSASFLYS | |
| ADSVKGRFTISADTSKNTAYLQMNSLRAED | | GVPSRFSGSGSGTDFTLTISSLQPEDFA | |
| TAVYYCARRHWPGGFDYWGQGTLVTVSS | | TYYCQQYLYHPATFGQGTKVEIKR | |
| (SEQ ID NO: 540) | | (SEQ ID NO: 541) | |
| CDR Sequences (Kabat) | | CDR Sequences (Kabat) | |
| CDR-H1 | DSWIH | CDR-L1 | RASQDVSTAVA |
| | (SEQ ID NO: 542) | | (SEQ ID NO: 545) |
| CDR-H2 | WISPYGGSTYYADSVKG | CDR-L2 | SASFLYS |
| | (SEQ ID NO: 543) | | (SEQ ID NO: 546) |
| CDR-H3 | RHWPGGFDY | CDR-L3 | QQYLYHPAT |
| | (SEQ ID NO: 544) | | (SEQ ID NO: 547) |
| CDR Sequences (IMGT) | | CDR Sequences (IMGT) | |
| CDR-H1 | GFTFSDSW | CDR-L1 | QDVSTA |
| | (SEQ ID NO: 548) | | (SEQ ID NO: 551) |
| CDR-H2 | ISPYGGST | CDR-L2 | SA |
| | (SEQ ID NO: 549) | | |
| CDR-H3 | ARRHWPGGFDY | CDR-L3 | QQYLYHPAT |
| | (SEQ ID NO: 550) | | (SEQ ID NO: 552) |
| CDR Sequences (Chothia) | | CDR Sequences (Chothia) | |
| CDR-H1 | GFTFSDS | CDR-L1 | RASQDVSTAVA |
| | (SEQ ID NO: 553) | | (SEQ ID NO: 556) |
| CDR-H2 | SPYGGS | CDR-L2 | SASFLYS |
| | (SEQ ID NO: 554) | | (SEQ ID NO: 557) |
| CDR-H3 | RHWPGGFDY | CDR-L3 | QQYLYHPAT |
| | (SEQ ID NO: 555) | | (SEQ ID NO: 558) |

TABLE 3B
PD-L1 Antibody Sequences (Avelumab)

| Heavy chain variable region | | Light chain variable region | |
|---|---|---|---|
| EVQLLESGGGLVQPGGSLRLSCAASGFTFS | | QSALTQPASVSGSPGQSITISCTGTSSD | |
| SYIMMWVRQAPGKGLEWVSSIYPSGGITFY | | VGGYNYVSWYQQHPGKAPKLMIYDVSNR | |
| ADTVKGRFTISRDNSKNTLYLQMNSLRAED | | PSGVSNRFSGSKSGNTASLTISGLQAED | |
| TAVYYCARIKLGTVTTVDYWGQGTLVTVSS | | EADYYCSSYTSSSTRVFGTGTKVTVLGQ | |
| (SEQ ID NO: 559) | | P (SEQ ID NO: 560) | |
| CDR Sequences (Kabat) | | CDR Sequences (Kabat) | |
| CDR-H1 | SYIMM | CDR-L1 | TGTSSDVGGYNYVS |
| | (SEQ ID NO: 561) | | (SEQ ID NO: 564) |
| CDR-H2 | SIYPSGGITFYADTVKG | CDR-L2 | DVSNRPS |
| | (SEQ ID NO: 562) | | (SEQ ID NO: 565) |
| CDR-H3 | IKLGTVTTVDY | CDR-L3 | SSYTSSSTRV |
| | (SEQ ID NO: 563) | | (SEQ ID NO: 566) |
| CDR Sequences (IMGT) | | CDR Sequences (IMGT) | |
| CDR-H1 | GFTFSSYI | CDR-L1 | SSDVGGYNY |
| | (SEQ ID NO: 567) | | (SEQ ID NO: 570) |
| CDR-H2 | IYPSGGIT | CDR-L2 | DV |
| | (SEQ ID NO: 568) | | |
| CDR-H3 | ARIKLGTVTTVDY | CDR-L3 | SSYTSSSTRV |
| | (SEQ ID NO: 569) | | (SEQ ID NO: 571) |
| CDR Sequences (Chothia) | | CDR Sequences (Chothia) | |
| CDR-H1 | GFTFSSY | CDR-L1 | TGTSSDVGGYNYVS |
| | (SEQ ID NO: 572) | | (SEQ ID NO: 575) |
| CDR-H2 | YPSGGI | CDR-L2 | DVSNRPS |
| | (SEQ ID NO: 573) | | (SEQ ID NO: 576) |
| CDR-H3 | IKLGTVTTVDY | CDR-L3 | SSYTSSSTRV |
| | (SEQ ID NO: 574) | | (SEQ ID NO: 577) |

TABLE 3C
PD-L1 Antibody Sequences (Durvalumab)

| Heavy chain variable region | | Light chain variable region | |
|---|---|---|---|
| EVQLVESGGGLVQPGGSLRLSCAASGFTFS | | EIVLTQSPGTLSLSPGERATLSCRASQR | |
| RYWMSWVRQAPGKGLEWVANIKQDGSEKYY | | VSSSYLAWYQQKPGQAPRLLIYDASSRA | |
| VDSVKGRFTISRDNAKNSLYLQMNSLRAED | | TGIPDRFSGSGSGTDFTLTISRLEPEDF | |
| TAVYYCAREGGWFGELAFDYWGQGTLVTVS | | AVYYCQQYGSLPWTFGQGTKVEIKRTV | |
| S (SEQ ID NO: 578) | | (SEQ ID NO: 579) | |
| CDR Sequences (Kabat) | | CDR Sequences (Kabat) | |
| CDR-H1 | RYWMS | CDR-L1 | RASQRVSSSYLA |
| | (SEQ ID NO: 580) | | (SEQ ID NO: 583) |
| CDR-H2 | NIKQDGSEKYYVDSVKG | CDR-L2 | DASSRAT |
| | (SEQ ID NO: 581) | | (SEQ ID NO: 584) |
| CDR-H3 | EGGWFGELAFDY | CDR-L3 | QQYGSLPWT |
| | (SEQ ID NO: 582) | | (SEQ ID NO: 585) |
| CDR Sequences (IMGT) | | CDR Sequences (IMGT) | |
| CDR-H1 | GFTFSRYW | CDR-L1 | QRVSSSY |
| | (SEQ ID NO: 586) | | (SEQ ID NO: 589) |
| CDR-H2 | IKQDGSEK | CDR-L2 | DA |
| | (SEQ ID NO: 587) | | |
| CDR-H3 | AREGGWFGELAFDY | CDR-L3 | QQYGSLPWT |
| | (SEQ ID NO: 588) | | (SEQ ID NO: 590) |
| CDR Sequences (Chothia) | | CDR Sequences (Chothia) | |
| CDR-H1 | GFTFSRY | CDR-L1 | RASQRVSSSYLA |
| | (SEQ ID NO: 591) | | (SEQ ID NO: 594) |
| CDR-H2 | KQDGSE | CDR-L2 | DASSRAT |
| | (SEQ ID NO: 592) | | (SEQ ID NO: 595) |
| CDR-H3 | EGGWFGELAFDY | CDR-L3 | QQYGSLPWT |
| | (SEQ ID NO: 593) | | (SEQ ID NO: 596) |

### 3. Fc Polypeptides

In some embodiments, the Fc polypeptide is a single chain Fc (scFc), which comprises two Fc chains linked together by a covalent linker, e.g., via an amino acid linker.

An IgG Fc chain (e.g., IgG1 Fc chain) typically contains two constant heavy domains (CH2 and CH3) and a hinge region connected to the CH2 domain.

In some embodiments, the Fc polypeptide comprises one or more IgG1 Fc chains (e.g., human IgG1 Fc chains or a human Fc chains), that is, the Fc polypeptide comprises an Fc chain that has an amino acid sequence that is substantially similar or identical to that of the chains within a wild type IgG1 Fc.

In some embodiments, the Fc polypeptide comprises one or more human IgG1 Fc chains; that is, the Fc polypeptide comprises an Fc chain that is substantially similar or identical to that of the Fc chains within a wild type human IgG1. In some embodiments, the wild type human IgG1 Fc has an amino acid sequence of SEQ ID NO:4.

In certain embodiments, fragment crystallizable (Fc) polypeptides comprise Fc chains that each have one or more mutations relative to a reference Fc chain of the same Ig class. As explained further herein below, the reference Fc chain may be of, e.g., the IgG1 class.

Unless otherwise noted, numbering of residues within an antibody fragment, e.g., an Fc chain, throughout this disclosure is according to the EU numbering.

In some embodiments, the Fc polypeptide comprises one or more human IgG1 Fc chains that is, except for mutations noted herein, the Fc polypeptide comprises an Fc chain that is substantially similar to that of the Fc chains within a wild type human IgG1.

In some embodiments, the Fc polypeptide comprises one or more IgG1 Fc chains (e.g., human IgG1 Fc chains or a human Fc chains), that is, except for having particular residue(s) (which may be different than the residue(s) in the corresponding wild type Fc chains) at certain positions as noted herein, the Fc polypeptide comprises an Fc chain that has an amino acid sequence that is substantially similar to that of the chains within a wild type IgG1 Fc. In some embodiments, the wild type IgG1 Fc is a human IgG1 Fc, in which each Fc chain has an amino acid sequence of SEQ ID NO:4.

For example, an Fc polypeptide may comprise an Fc chain with an amino acid sequence that is at least 85%, at least 87.5%, at least 90%, at least 91%, at least 92%, at least 93%, at least 94%, at least 95%, at least 96%, at least 97%, at least 98%, or at least 99% identical to that of an Fc chain within a wild-type IgG1 Fc. In some embodiments, an Fc polypeptide comprises an Fc chain that comprises the particular residue(s) at certain position(s) specifically described for that Fc chain, but has an amino acid sequence that is otherwise 100% identical to a corresponding Fc chain within a wild type Fc chain, e.g., wild type IgG1 Fc chain. In some embodiments, the Fc polypeptide comprises an Fc chain that has an amino acid sequence that differs by at least one, at least two, at least three, or at least four amino acid residues from the sequence of SEQ ID NO:4. In some embodiments, the Fc polypeptide comprises an Fc chain that has an amino acid sequence that differs by no more than ten, no more than nine, no more than eight, no more than seven, no more than six, no more than five, or no more than four amino acid residues from the sequence of SEQ ID NO:4.

In some embodiments, the Fc chain comprise one or more IgG1, IgG2, IgG3, or IgG4 heavy chain constant regions, e.g., one or more heavy chain constant region having at least 93%, at least 94%, at least 95%, at least 96%, at least 97%, at least 98%, at least 99%, or identical to that of SEQ ID NO:599, 600, 601, or 602.

In some embodiments, the Fc polypeptide comprises an “effector silent” Fc chain that is not capable of binding, or only binds at substantially decreased levels, to one or more Fcγ receptors (e.g., human Fcγ receptors hFcγRI, hFcγRIIa, hFcγRIIb, hFcγRIIIa, hFcγRIIIb). In some embodiments, the Fc polypeptide comprises an Fc chain with a half-life extending mutation or set of mutations.

For example, in some embodiments, the Fc polypeptide comprises an IgG1 Fc with an “LLRAL” mutation: L234A, L235A, G236R, G237A, and A330L, with the amino acid residue at position P329 being proline (same as in the wild type)).

### 4. Linkers

In certain embodiments, linkers are used within fusion polypeptides and/or within single-chain molecules such as scFcs. In some embodiments, the linker is an amino acid linker. For example, a linker as employed herein may comprise from about 1 to about 100 amino acid residues, e.g., about 1 to about 70, about 2 to about 70, about 1 to about 30, or about 2 to about 30 amino acid residues. In some embodiments, the linker comprises at least 2, at least 3, at least 4, at least 5, at least 6, at least 7, at least 8, at least 9, or at least 10 amino acid residues.

In certain embodiments, the linker comprises a glycine-serine sequence, e.g., a (GₙS)ₘ sequence (e.g., GGS, GGGS (SEQ ID NO: 6), and/or GGGGS (SEQ ID NO: 7) sequence) that is present in at least 1, at least 2, at least 3, at least 4, at least 5, at least 6, at least 7, at least 8, at least 9, at least 10, at least 11, at least 12, at least 13, or at least 14 copies within the linker.

### B. Self-Assembled Polypeptide Complexes

In one aspect, provided are self-assembled polypeptide complexes comprising a plurality of fusion polypeptides as disclosed herein. Generally, provided self-assembled polypeptide complexes comprise (a) a plurality of TNF receptor-binding fusion polypeptides, each TNF receptor-binding fusion polypeptide comprising (1) a TNF-receptor binding moiety linked to (2) a nanocage monomer or subunit thereof, and (b) a plurality of Fc fusion polypeptides, each Fc fusion polypeptide comprising (1) an Fc polypeptide linked to (2) a nanocage monomer or subunit thereof, wherein the Fc polypeptide comprises an Fc chain.

In some embodiments, the nanocage monomer is a ferritin monomer, and each fusion polypeptide within the self-assembled polypeptide complex comprises a ferritin light chain or a subunit of a ferritin light chain. In these embodiments, the self-assembled polypeptide complex does not comprise any ferritin heavy chains, subunits of ferritin heavy chains, or other ferritin components capable of binding to iron or capable of ferroxidase activity.

In some embodiments, the nanocage monomer or subunit thereof is a ferritin monomer subunit, and (a) each TNF receptor-binding fusion polypeptide comprises a ferritin monomer subunit which is C-half-ferritin and each Fc fusion polypeptide comprises a ferritin monomer subunit which is N-half-ferritin; or (b) each TNF receptor-binding fusion polypeptide comprises a ferritin monomer subunit which is N-half ferritin and each Fc fusion polypeptide comprises a ferritin monomer subunit which is C-half-ferritin.

In some embodiments, the self-assembled polypeptide complex comprises between 24 and 48 fusion polypeptides in total. In some embodiments, the self-assembled polypeptide complex comprises 24 fusion polypeptides in total. In some embodiments, the self-assembled polypeptide complex comprises more than 24 fusion polypeptides, e.g., at least 26, at least 28, at least 30, at least 32 fusion polypeptides, at least 34 fusion polypeptides, at least 36 fusion polypeptides, at least 38 fusion polypeptides, at least 40 fusion polypeptides, at least 42 fusion polypeptides, at least 44 fusion polypeptides, at least 46 fusion polypeptides, or at least 48 fusion polypeptides in total. In some embodiments, the self-assembled polypeptide complex comprises about 32 fusion polypeptides.

In some embodiments, the self-assembled polypeptide complex comprises at least two, at least three, at least four, at least five, least six, at least seven, or at least eight TNF receptor-binding fusion polypeptides.

In some embodiments, the self-assembled polypeptide complex comprises at least two, at least three, at least four, at least five, least six, at least seven, or at least eight Fc fusion polypeptides.

In some embodiments, the self-assembled polypeptide complex further comprises at least two, at least three, at least four, at least five, least six, at least seven, at least eight, at least nine, at least ten, least 11, at least 12, at least 13, at least 14, at least 15, or at least 16 fusion polypeptides distinct from the TNF receptor-binding fusion polypeptide and the Fc fusion polypeptide, e.g., a tumor-binding fusion polypeptide (e.g., a fusion polypeptide comprising a tumor-binding moiety linked to nanocage monomer or a subunit thereof).

In some embodiments, the self-assembled polypeptide complex comprises a ratio of approximately 1:1, 11:13, 3:5, 1:2, 7:17, 1:3, 2:7, 5:19, 1:4, 1:5, 1:6, 1:7, 1:8, 1:12, 1:24 of TNF receptor-binding fusion polypeptides to all other fusion polypeptides.

In some embodiments, the self-assembled polypeptide complex comprises a ratio of approximately 1:1, 11:13, 3:5, 1:2, 7:17, 1:3, 2:7, 5:19, 1:4, 1:5, 1:6, 1:7, 1:8, 1:12, 1:24 of TNF receptor-binding fusion polypeptides to Fc fusion polypeptides.

### Pharmacokinetic Characteristics

In certain embodiments, when administered to a subject in need thereof, a provided self-assembled polypeptide complex, has one or more pharmacokinetic features similar to that of a reference IgG molecule (e.g., an IgG molecule whose class matches the class of an Fc chain within an Fc polypeptide of an Fc fusion polypeptide within the self-assembled polypeptide complex).

### Effects

In certain embodiments, a provided self-assembled polypeptide complex is capable of activating a TNF receptor, e.g., inducing clustering of the TNF receptor and/or inducing downstream signals from the TNF receptor. In some embodiments, a provided self-assembled polypeptide complex exhibits agonistic activity at a TNF receptor which is independent of Fc crosslinking and/or Fc effector functions.

### C. Methods of Treatment

In one aspect, provided are methods that may be useful for treating, ameliorating, or preventing a disease or a condition, generally comprising a step of administering a self-assembled polypeptide complex of the present disclosure (or a composition thereof) to a subject.

In some embodiments, the subject is a mammal, e.g., a human.

Compositions for administration to subjects generally comprise a self-assembled polypeptide complex as disclosed herein. In some embodiments, such compositions further comprise a pharmaceutically acceptable excipient.

Compositions may be formulated for administration for any of a variety of routes of administration, including systemic routes (e.g., oral, inhalation, intranasal, intravenous, intraperitoneal, subcutaneous, or intramuscular administration).

In some embodiments, the step of administering results in improvement in one or more clinical outcomes or metrics in the subject.

For example, in some embodiments, the subject has cancer, e.g., a cancer characterized by an immunologically cold tumor. Examples of cold tumors include, without limitation, glioblastomas, ovarian cancer, prostate cancer, pancreatic cancer, and breast cancer tumors that are characterized by a lack of T cell infiltration.

In some embodiments, the step of administering results in slowing or inhibiting progression of the tumor, e.g., regression of the tumor. In some embodiments, the step of administering results in complete regression of the tumor.

### EXAMPLES

### Example 1. Construction and Expression of α-4-1BB Multabodies (MBs)

This Example describes the generation of α-4-1BB Multabodies (MBs) comprising a combination of fusion proteins comprising a (1) human ferritin light chain or subunit thereof and (2) a single-chain Fab (scFab) or a single chain Fc-dimer (scFc), fused via a linker, such as a (Glyₙ-Ser)ₘ amino acid linker as described herein.

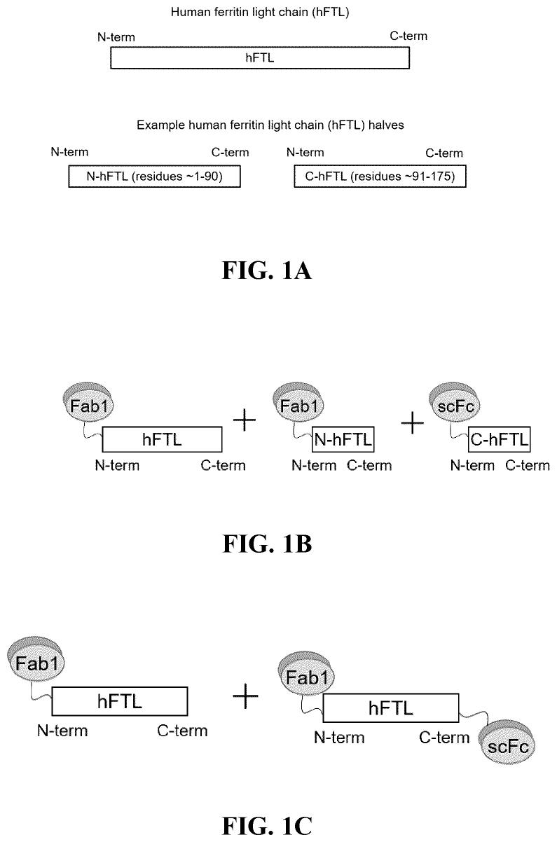

Genes encoding fusion proteins (1) scFab of an α-4-1BB fused to the N-terminus of full-length ferritin, (2) scFab of an α-4-1BB fused to the N-terminus of an N-half ferritin, and (3) scFc fused to the N-terminus of a C-half ferritin were prepared, mixed at a molar ratio of 2:1:1 and transiently transfected into ExpiCHO-S cells for the production and formation of the 4-1BB-targeting MBs (FIG. 1B).

Eight different MBs were made, each using an scFab of a different 4-1BB IgG. Three MBs were made using scFabs based on urelumab, NM21, and clone A1, which bind to the distal-most CRD domain (CRD1) of 4-1BB or an epitope between CRD1 and CRD2 of 4-1BB; one MB was made using scFab based on AG10058, which binds to CRD2 of 4-1BB; and four MBs were made using scFabs based on ABLPNB.01, ABLPNB.03, and ABLPNB.07, which bind to CRD4 or an epitope between CRD3 and CRD4 of 4-1BB. (ABLPNB.01, ABLPNB.03, and ABLPNB.07 are sequences that correspond to a putative monoclonal antibody ABL503.

The scFc-ferritin fusion proteins comprised engineered IgG1 Fc chains which contained the “LLRAL” mutation (L234A, L235A, G236R, G237A, and A330L; with the amino acid residue at position P329 being proline (same as in the wild type)), as shown in Table 1 below. Numbering in Table 5 is according to the EU numbering scheme. This “LLRAL” combination of mutations render the IgG1 Fc “silent” with respect to effector activity, consistent with significantly diminished or no binding to Fcγ receptors (data not shown).

TABLE 5
Residues at certain positions within IgG1 Fc chains used in
MBs and corresponding SEQ ID NOs of scFc-C hFTL constructs
In rows corresponding to sets of engineered Fc chains, residues
are only shown at positions that differ from the wild type residue.

| | Amino acid position → | | | | | | |
|---|---|---|---|---|---|---|---|
| | | | | | | | SEQ ID NO: of |
| | | | | | | | corresponding |
| | | | | | | | scFc-C_hFTL |
| | 234 | 235 | 236 | 237 | 329 | 330 | construct |
| WT | L | L | G | G | P | A | SEQ ID |
| (wild type) | | | | | | | NO: 597 |
| LLRAL | A | A | R | A | | L | SEQ ID |
| | | | | | | | NO: 598 |

### Example 2. Target Binding of MBs Determined by Biolayer Interferometry

The binding kinetics and affinity of exemplary α-4-1BB MBs generated as described in Example 1 to 4-1BB can be determined by biolayer interferometry (BLI). For example, the binding characteristics of IgG antibodies (from which the scFabs within the 4-1BB MBs are derived) can also be determined for comparison.

For example, Ni-NTA biosensors can be coated with His-tagged antigens (e.g., 4-1BB). Coated biosensors can be dipped into wells containing serial dilutions of the MBs (or of the antibody controls) in a buffer such as a phosphate-buffered saline (PBS) buffer optionally comprising Tween 20 and optionally comprising bovine serum albumin (BSA) during an association phase and during a dissociation phase.

Biosensors can be regenerated between experiments, for example, by applying a glycine solution several times and by recharging in NiSO₄.

Target binding can be evaluated based on the maximal association binding response at the end of the association phase, the dissociation rate (k_{off}), and/or the equilibrium dissociation constant (K_{D}) calculated using a 1:1 fitting model. A maximal association binding response of less than 0.1 nm for a concentration of 20 nM MB can be classified as “non-binding.”

### Example 3. Binding of MBs to Fc Receptors Determined by Biolayer Interferometry

The binding kinetics and affinities of various MBs generated as described in Example 1 to various Fc receptors can be determined by biolayer interferometry.

MBs tested in this Example contain polypeptides comprising one or more Fc chains. As controls for comparison, MBs without Fc-containing polypeptides and/or IgG antibodies of the same class as the Fc chains used in the MBs can be used.

Binding to various Fc receptors can be determined, for example, to human Fc receptors, such as: human Fc gamma receptor type I (hFcγRI), hFcγRIIa, hFcγRIIb, hFcγRIIIa, hFcγRIIIb, and human neonatal Fc receptor (hFcRn). To assess the possibility and relevance of testing the MBs in animal models, binding to non-human Fc receptors can also be assessed, such as to cynomolgus Fc receptors (e.g., cynomolgus FcγRI (cFcγRI), cFcγRIIa, cFcγRIIb, cFcγRIII, and cFcRn) and/or to mouse Fc receptors (e.g., mouse FcγRI (mFcγRI), mFcγRIIb, mFcγRIII, mFcγRIV, and mFcRn).

Experiments can be performed similarly as described in Example 2, except that His-tagged Fc receptors are coated onto Ni-NTA biosensors and titrated with test MBs at various concentrations. To assess the potential of the MBs to undergo endosomal recycling, binding to FcRn can be measured at pH 6.0 for association and at pH 7.4 for dissociation.

### Example 4. Assessment of 4-1BB Agonism by I2G Antibodies

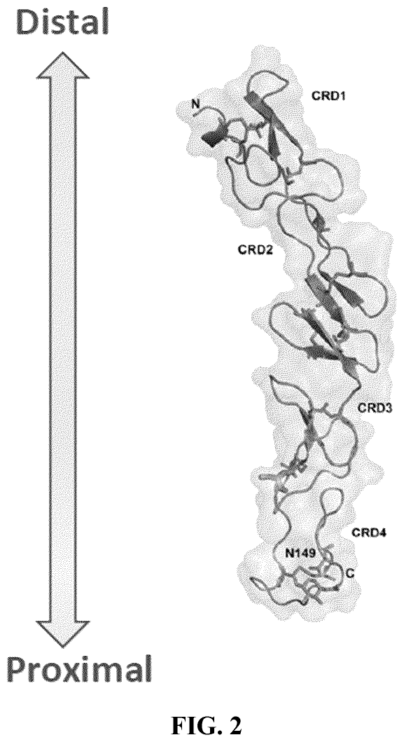

This Example describes experiments conducted to characterize 4-1BB agonism activity by urelumab and utomilumab, two antibodies that bind to different domains of 4-1BB. Urelumab, an IgG4 antibody, binds to the CRD-1 domain, the domain of 4-1BB most distal to the membrane. Utomilumab is an IgG2 antibody that binds to a 4-1BB epitope within CRD-3 and CRD-4, which are located more proximally to the cell membrane (FIG. 2.) An IgG1 version of utomilumab was also used for experiments described in this Example.

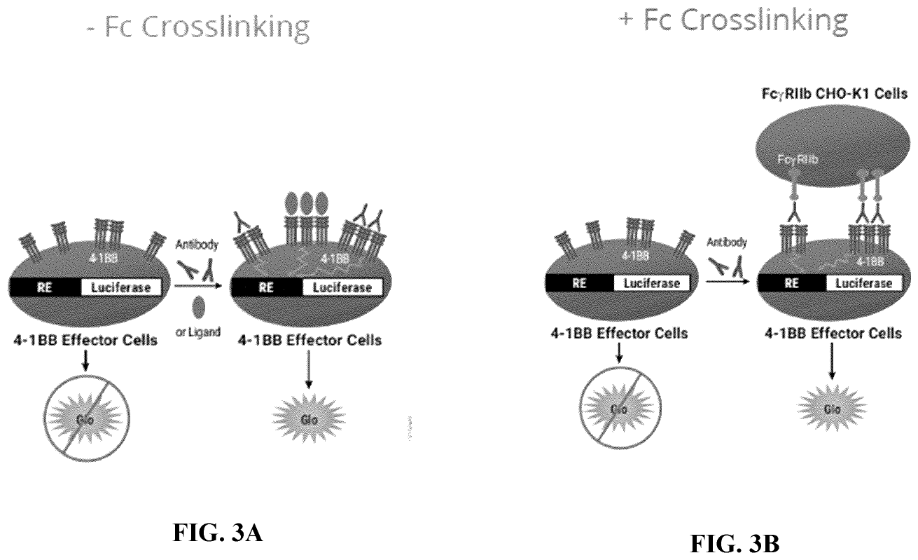

4-1BB antibodies were tested in in vitro 4-1BB reporter assays, alongside human 4-1BBL and a class-matched IgG control which does not bind to 4-1BB. The reporter assay uses engineered 4-1BB-expressing effector cells containing a luciferase transgene driven by a promoter responsive to downstream signals resulting from 4-1BB multimerization. These cells can be used without additional cells to assess agonism in the absence of Fc crosslinking. (FIG. 3A). To assess Fc crosslinking-dependent agonism, engineered FcγRIIb CHO-K1 cells are used together with the 4-1BB effector cells and the test agent (FIG. 3B.)

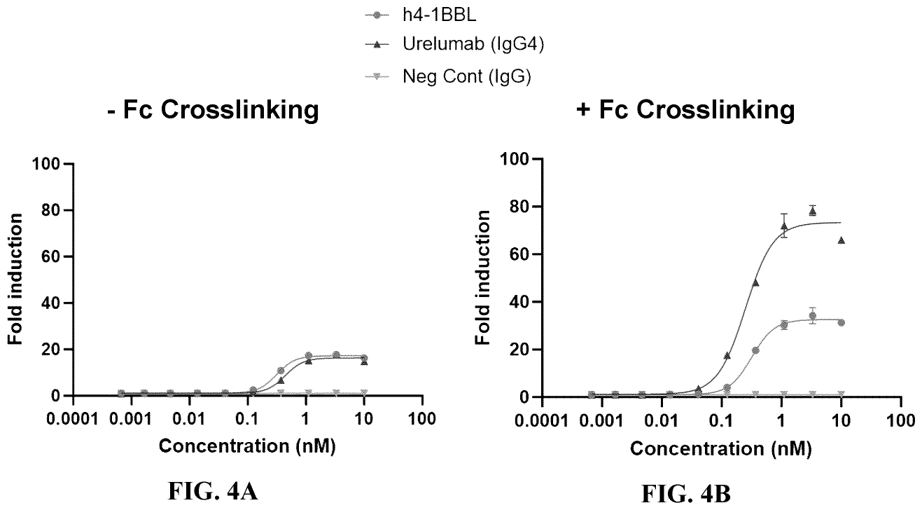

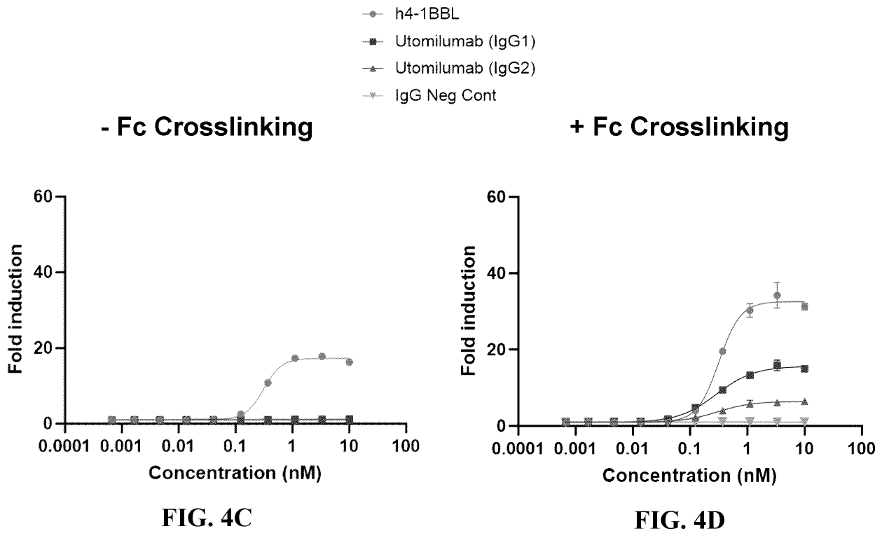

FIGS. 4A and 4B show the results for urelumab in the absence or presence, respectively, of Fc crosslinking by FcγRIIb CHO-K1 cells. FIGS. 4C and 4D show the results for utomilumab in the absence or presence, respectively, of Fc crosslinking.

Urelumab exhibited strong 4-1BB agonism activity in this assay, but its agonism was largely dependent on Fc crosslinking. Urelumab exhibited low but detectable levels of 4-1BB agonism in the absence of Fc crosslinking (FIG. 4A) which was greatly enhanced in the presence of Fc crosslinking (FIG. 4B). By contrast, utomilumab was a much weaker agonist, exhibiting no detectable agonism in the absence of Fc crosslinking (FIG. 4C) and some level of agonism in the presence of Fc crosslinking (FIG. 4D), though at much lower levels than that exhibited by urelumab.

### Example 5. Fc-Independent Agonism by α-4-1BB MBs

This Example describes experiments conducted to characterize the 4-1BB agonism activity of the α-4-1BB MBs which were generated as described in Example 1. While these α-4-1BB MBs display IgG1 Fc, the displayed Fc contains “LLRAL” mutations (as explained in Example 1), rendering the MBs effector silent as a result of their inability (or significantly diminished ability) to bind to Fcγ receptors.

Effector-silent α-4-1BB MBs which display scFabs based on AG10058 were tested using the same kind of in vitro 4-1BB luciferase reporter assay described in Example 4. For comparison, human 4-1BBL, urelumab (IgG), AG10058 IgG, a negative control MB (not comprising α-4-1BB Fabs), and a class-matched IgG control which does not bind to 4-1BB were also tested.

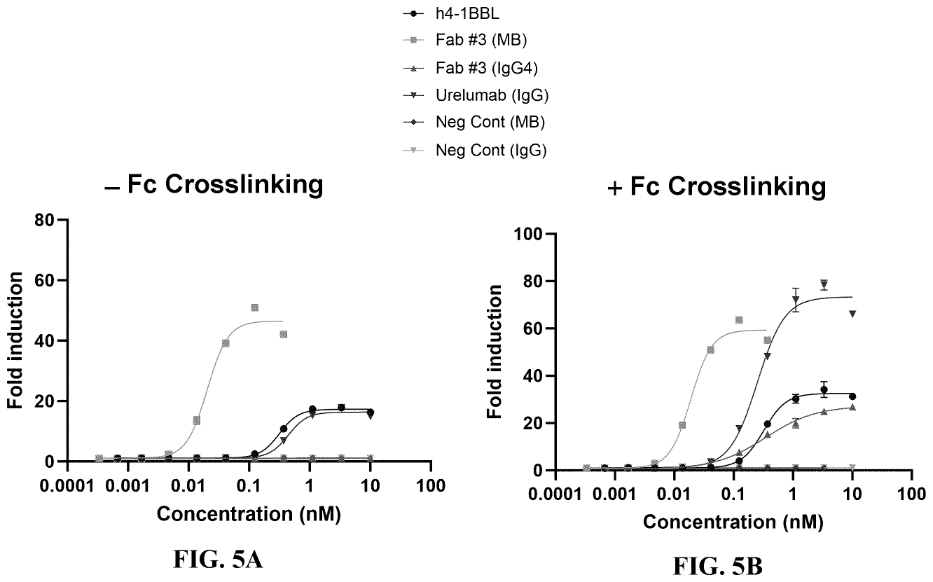

FIGS. 5A and 5B show the results for these experiments in the absence or presence, respectively, of Fc crosslinking by FcγRIIb CHO-K1 cells. While the human 4-1BBL, urelumab IgG, and AG10058 IgG exhibited Fc crosslinking-dependent 4-1BB agonism (compare the low or undetectable amount of induction in FIG. 5A to the significantly higher levels of induction in FIG. 5B), effector silent 4-1BB MBs exhibited strong induction in the absence of Fc crosslinking (FIG. 5A).

Thus, effector silent 4-1BB MBs were able to agonize 4-1BB and induce a strong response even in the absence of Fc crosslinking such as that provided by cells expressing Fcγ receptors. These results demonstrate that an IgG antibody which does not exhibit Fc-independent 4-1BB agonism can be “transformed” into an Fc-independent 4-1BB agonist by displaying a Fab based on the same antibody as part of a MB of the present disclosure. Moreover, the Fc-independent 4-1BB agonism exhibited by α-4-1BB MBs of the present disclosure suggest that α-41BB MBs may potentially treat immunologically cold tumors which are very sparsely infiltrated with FcγR-expressing cells.

### Example 6. Pharmacokinetics of MBs in a Non-Human Animal Models

The pharmacokinetics (PK) of exemplary α-4-1BB MBs generated as described in Example 1 can be analyzed in one or more non-human animal models, such as mice and/or non-human primates (e.g., cynomolgus monkeys).

Animals receive a single bolus injection of the MB or a control molecule (e.g., an IgG) by systemic route of administration (e.g., intravenous). Blood samples are collected at various timepoints, e.g., at around of 3 h, 24 h, 48 h, 72 h, 5 days, 7 days, 14 days, 21 days, and 28 days after injection.

Multi-dose studies can also be performed, with the first and second doses spaced apart, e.g., by a number of days or weeks.

Plasma or serum samples may be obtained from blood samples and stored frozen until use.

To measure levels of MBs or control molecules in plasma or serum by enzyme-linked immunosorbent assay (ELISA), recombinant 4-1BB is coated onto plates and left overnight at 4° C. After washing twice (e.g., with a solution such as phosphate-buffered saline (PBS)-Tween-20, the plate is blocked with a bovine serum albumin (BSA) solution in PBS for 1 hour at room temperature and subsequently washed twice. Plasma or serum samples are diluted in PBS/Tween-20/BSA solution, added to wells, and incubated for 1 h at room temperature on a shaker. After another wash step, bound molecules are detected by incubation with diluted goat polyclonal α-human Fc-HRP secondary antibody. After a further wash step, a substrate used for detection is added, and the absorbances at 450 nm were read using a microplate reader. A calibration curve is prepared using dilutions of MBs in PBS/Tween-20/BSA solution.

Half-lives of MBs in animals can be calculated based on these measurements, and the half-lives of MBs can be compared to those of IgG molecules.

### Example 7. Safety and Therapeutic Efficacy of α-4-1BB MBs in a Xenograft Mouse Model

The safety and therapeutic effect of exemplary MBs can be evaluated in a colorectal cancer (CRC) Xenograft model.

Mouse models. A number of humanized CRC tumor-grafted mouse models which can be used to assess in vivo safety and therapeutic efficacy of α-4-1BB MBs are disclosed herein. For example, in one model, C57BL/6 mice with a human 4-1BB/4-1BBL double knock-in are used. 2×10⁶ colorectal cancer cells (e.g., MC38 or CT26 cells) can be mixed with Corning Matrigel in a 1:1 volume ratio and then inoculated subcutaneously into 8-week old 4-1BB/4-1BBL double knock-in mice.

In another model, an immunodeficient B-NDG B2m knockout plus mouse, transplanted human peripheral blood mononuclear cells (PBMCs), is used. 2×10⁶ Colo-205 colorectal cancer cells mixed with 1:1 Matrigel can be inoculated subcutaneously into B-NDG B2m KO plus mice.

After palpable tumors are established, the mice can be randomized on the basis of tumor volume and body weight. Tumor volume can be estimated by measuring with calipers and using the following equation: volume=0.5×length×width×width.

Treatment: Mice can be administered an α-4-1BB MB of the present disclosure or a control (e.g., an α-4-1BB IgG), an IgG isotype control, and/or a control MB that does not bind to α-4-1BB), for example, by intraperitoneal injection, according to a dosing schedule to be determined by the investigator.

Assessment of therapeutic effect: Tumor growth, survival curves and weight loss can be compared across treatment groups. Reduction in tumor growth and/or improved survival in animals treated with MBs (as compared with those treated with control molecules) would suggest improved efficacy of α-4-1BB MBs.

Toxicology study: Toxicity studies can be conducted in humanized mouse model (as described above) and/or in cynomolgus monkeys. Humanized 4-1BB mice can be intraperitoneally injected with different dose amounts of α-4-1BB MBs. Cynomolgus monkeys can be administered different dose amounts of α-4-1BB MBs by intravenous or intraperitoneal injection. Mouse necropsies can be performed, and the major organs collected for histological evaluation. All the tissues can be fixed (for example, in 10% neutral-buffered formalin), processed, embedded in paraffin, sectioned, stained with hematoxylin and eosin (H&E), and analyzed by a pathologist. In addition, blood and serum can also be collected for clinical hematological and chemical analyses, for example, including biomarkers of liver function.

Reduced toxicity (such as liver toxicity) in animals treated with α-4-1BB MBs would indicate that α-4-1BB MBs of the present disclosure have an improved safety profile.

### Example 8. Safety and Therapeutic Efficacy of α-4-1BB MBs in a Xenograft Mouse Model of a Cold Tumor

Experiments similar to those described in Example 7 can be performed using an immunologically cold tumor model, such as 4T1 or B16F10 tumor models.

### Example 9. Epitope Binning by Competition Assay

The epitopes of the α-4-1BB IgGs were categorized (or binned) using a competition assay.

Using the in-tandem competition assay format on an Octet RED96 Biolayer Interferometer, IgGs and 4-1BBL were prepared at 200 nM (P #1) and 100 nM (P #2). NiNTA biosensors were loaded with His-tagged 4-1BB protein and dipped into saturating conditions of P #1 to allow binding, followed by P #2 to assess competition. Competing pairs were identified when binding of P #2 was blocked. Non-competing pairs were identified when binding of P #2 was permitted in the presence of bound P #1. All IgGs and 4-1BBL were tested in both binding orientations (P #1 and P #2) and a subsequent matrix of competing versus non-competing pairs was generated.

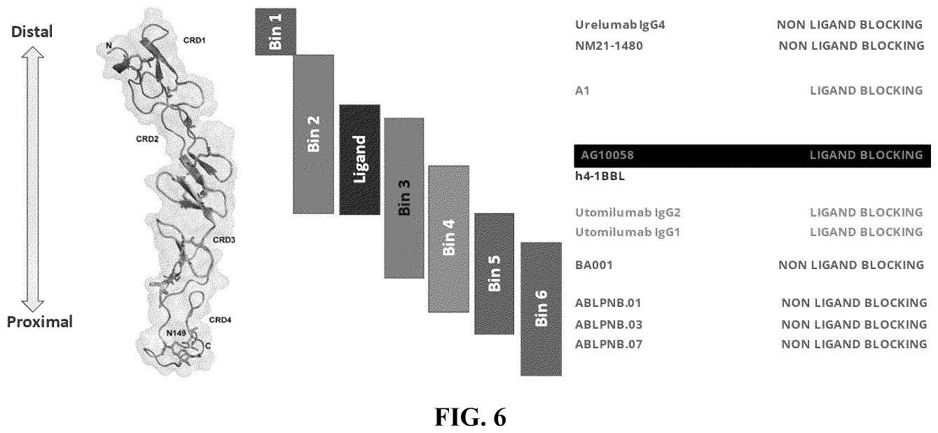

Distinct epitope bins were identified and mapped to 4-1BB as shown in FIG. 6.

### Example 10. Assessment of 4-1BB Agonism by α-4-1BB MBs

This Example describes experiments conducted to characterize the 4-1BB agonism activity of the α-4-1BB MBs which were generated as described in Example 1. While these α-4-1BB MBs display IgG1 Fc, the displayed Fc contains “LLRAL” mutations (as explained in Example 1), rendering the MBs effector silent as a result of their inability (or significantly diminished ability) to bind to Fcγ receptors.

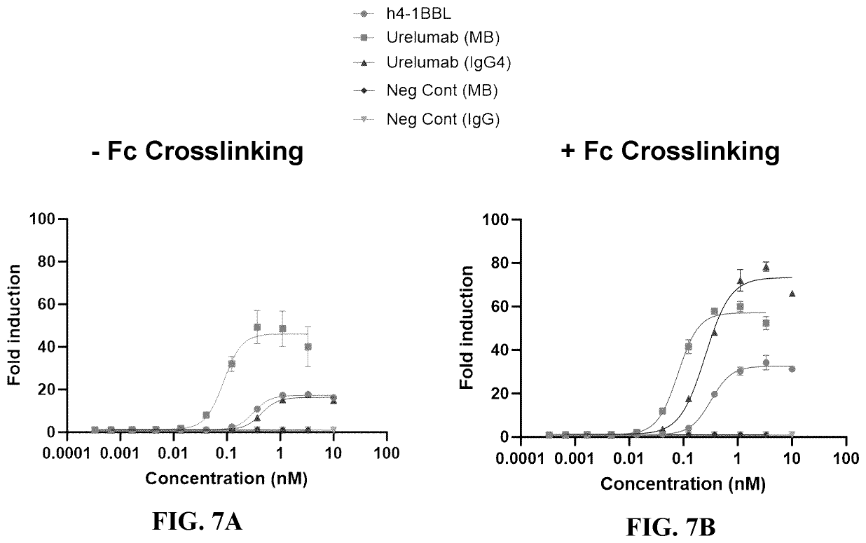

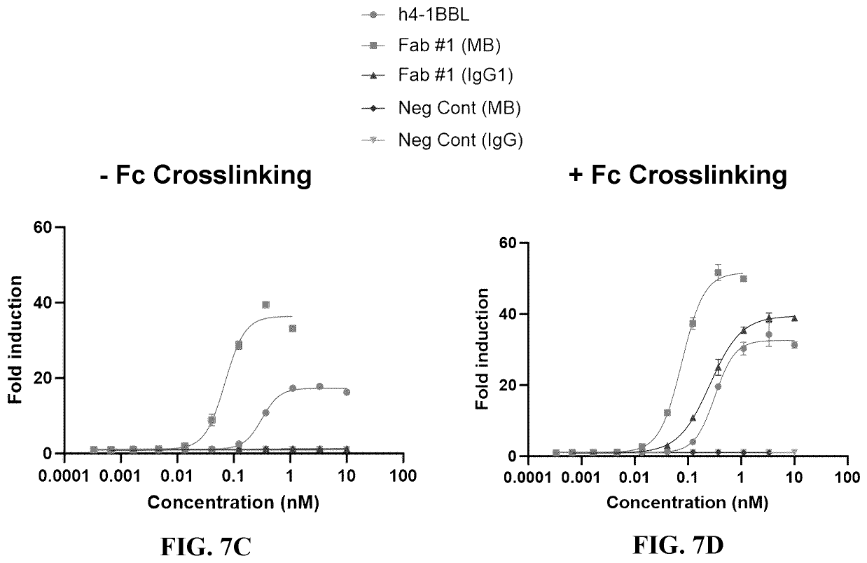

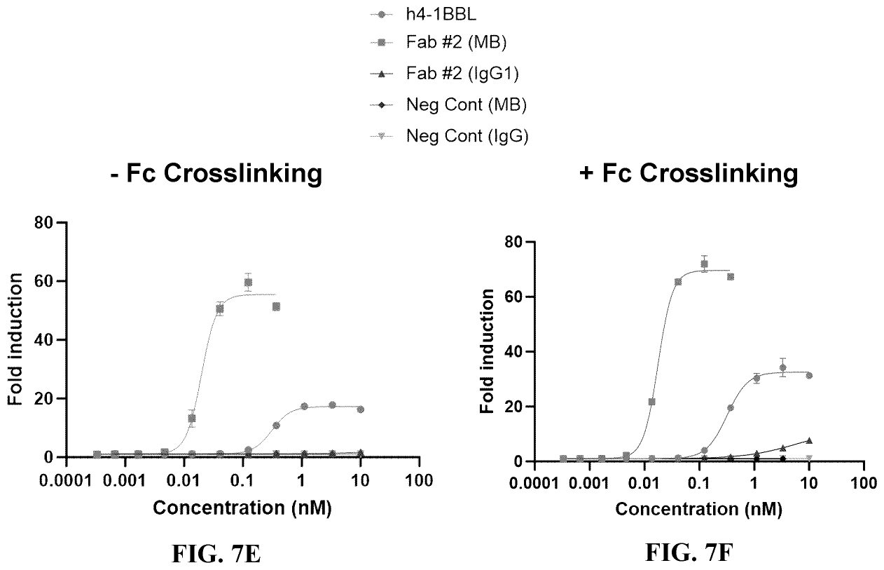

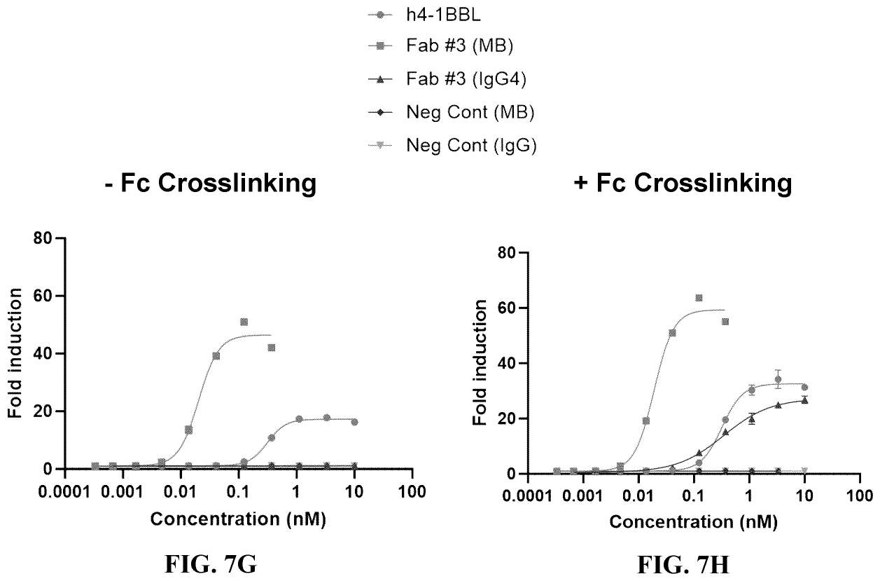

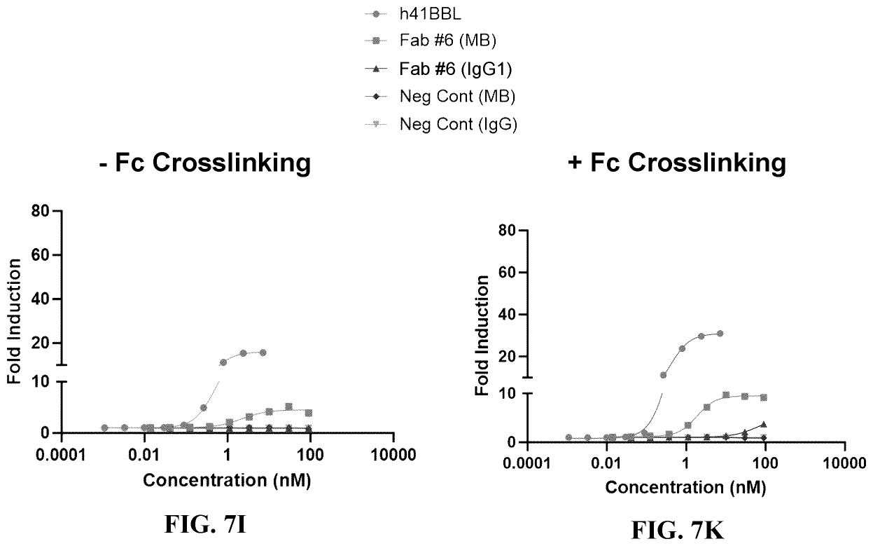

Effector-silent α-4-1BB MBs which display scFabs based on different scFabs (as described in Example 1) were tested using the same kind of in vitro 4-1BB luciferase reporter assay described in Example 4. FIGS. 7A and 7B show the results for urelumab in the absence or presence, respectively, of Fc crosslinking by FcγRIIb CHO-K1 cells. FIGS. 7C and 7D show results for NM21 (Fab #1) in the absence or presence, respectively, of Fc crosslinking. FIGS. 7E and 7F show results for A1 (Fab #2) in the absence or presence, respectively, of Fc crosslinking. FIGS. 7G and 7H show results for AG10058 (Fab #3) in the absence or presence, respectively, of Fc crosslinking. FIGS. 71 and 7K show results for ABLPNB.03 (Fab #6) in the absence or presence, respectively, of Fc crosslinking.

While urelumab, NM21, and AG10058 IgGs exhibited Fc crosslinking-dependent 4-1BB agonism (compare the low or undetectable amount of induction in FIGS. 7A, 7C, and 7G to the significantly higher levels of induction in FIGS. 7B, 7D, and 7H), effector silent α-4-1BB MBs of NM21 (Fab #1; FIG. 7C), A1 (Fab #2; FIG. 7E), and AG10058 (Fab #3; FIG. 7G) exhibited strong 4-1BB agonism in the absence of Fc crosslinking. The 4-1BB agonism of A1 (Fab #2) and ABLPNB.03 (Fab #6) IgGs did not significantly improve with Fc crosslinking, as observed by the comparable amounts of induction (compare IgG1 in FIGS. 7E and 7F for A1, and IgG in FIGS. 7I and 7K for ABLPNB.03). In addition, effector silent α-4-1BB MBs of ABLPNB.03 (Fab #6) exhibited weak 4-1BB agonism both in the absence or presence of Fc crosslinking (FIGS. 7I and 7K, respectively).

Table 6 depicts the half maximal effective concentrations (EC₅₀) for the antibodies which were tested in this Example.

TABLE 6
EC₅₀ values of 4-1BB agonism induced by antibodies
and MBs exemplified in FIGs. 7A-7K, in the
absence or presence of Fc crosslinking.

| ID | EC₅₀ (nM) − Fc | EC₅₀ (nM) + Fc |
|---|---|---|
| 4-1BBL | 0.315 | 0.320 |
| Urelumab IgG4 | 0.442 | 0.250 |
| Urelumab MB | 0.087 | 0.079 |
| Utomilumab IgG1 | — | 0.287 |
| Fab #1 MB | 0.070 | 0.077 |
| Fab #1 IgG | — | 0.258 |
| Fab #2 MB | 0.020 | 0.018 |
| Fab #2 IgG | — | — |
| Fab #3 MB | 0.020 | 0.019 |
| Fab #3 IgG | — | 0.346 |
| Fab #6 MB | 2.220 | 1.879 |
| Fab #6 IgG | — | — |
| Neg cont MB | — | — |
| Neg cont IgG | — | — |

“—” denotes EC₅₀ values that were not measurable.

### Example 11. Assessment α-4-1BB IgGs and α-4-1BB MBs for Cross-Reactivity to Human, Cynomolgus Monkey, and Mouse 4-1BB

The capacity of the α-4-1BB IgGs and α-4-1BB MBs to cross-react to human, cynomolgus monkey, and mouse 4-1BB was evaluated.

Cross-reactivity was assessed using ELISA. Briefly, plates were coated with recombinant 4-1BB proteins derived from either human, cynomolgus monkey, or mouse. Plates were then washed, blocked, and incubated with bovine serum albumin (BSA), along with a series of known concentrations of α-4-1BB IgGs/MBs. The plate was then washed and incubated with a Goat α-human Fcy-HRP secondary antibody to detect bound IgG/MB. The plates were then washed again and developed with TMB substrate to detect HRP and read at OD 450 nm. Cross-reactivity to human, cynomolgus monkey, and mouse 4-1BB proteins was evaluated for all of the antibodies described in FIG. 6 and MBs based thereon. All α-4-1BB MBs and corresponding IgGs evaluated exhibited cross-reactivity to human and cynomolgus monkey 4-1BB proteins. AG10058 IgG and MB also exhibited cross-reactivity to mouse 4-1BB.

### Example 12. Therapeutic Efficacy of α-4-1BB MBs in a XenogRaft Mouse Model

The therapeutic efficacy of exemplary MBs was evaluated in a colorectal cancer (CRC) Xenograft model.

Mouse models: C57BL/6 mice with a human 4-1BB/4-1BBL double knock-in (“Hu4-1BB KI mice”) were intradermally injected with 1.0×10⁶ colorectal cancer cells (MC38 cells). After 5 days, the mice were randomized on the basis of tumor volume. Treatment: Five days after inoculation with tumor cells, α-4-1BB treatment was administered by intraperitoneal (IP) injections once per week for 2 weeks.

Assessment of therapeutic effect: Throughout the study, non-terminal blood was sampled, and tumor size/body weight was measured 3 times per week. Tumor volume was estimated by measuring with calipers and using the following equation: volume=0.5×length×width×width.

Humane endpoint: Mice reached humane endpoint when tumors grew to a size of >15 mm in length or width.

Histopathology: Liver tissue was harvested at endpoint and subjected to histopathology (Formalin-Fixed, Paraffin-Embedded (FFPE) preparation).

### Results

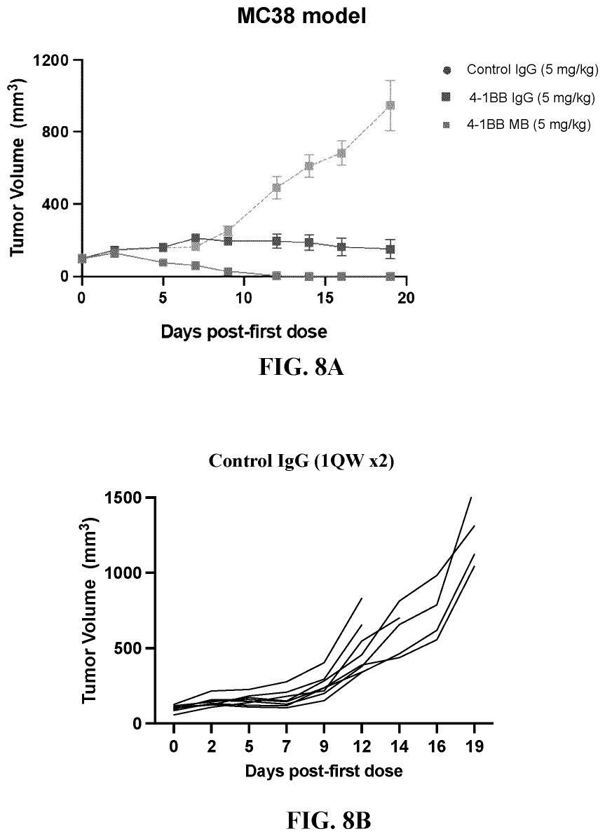

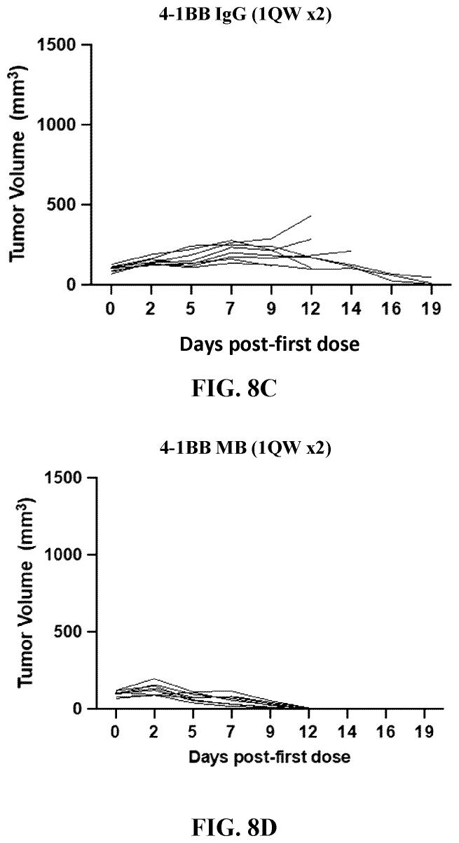

While both α-4-1BB IgG and α-4-1BB MB demonstrated in vivo efficacy compared to the control group, treatment with α-4-1BB MB resulted in superior efficacy compared to the parent α-4-1BB IgG. 10% (1 in 10) of the mice treated with α-4-1BB IgG showed complete tumor regression (FIG. 8C), while 100% (8 out of 8) of the mice treated with α-4-1BB MB showed complete tumor regression (FIG. 8D).

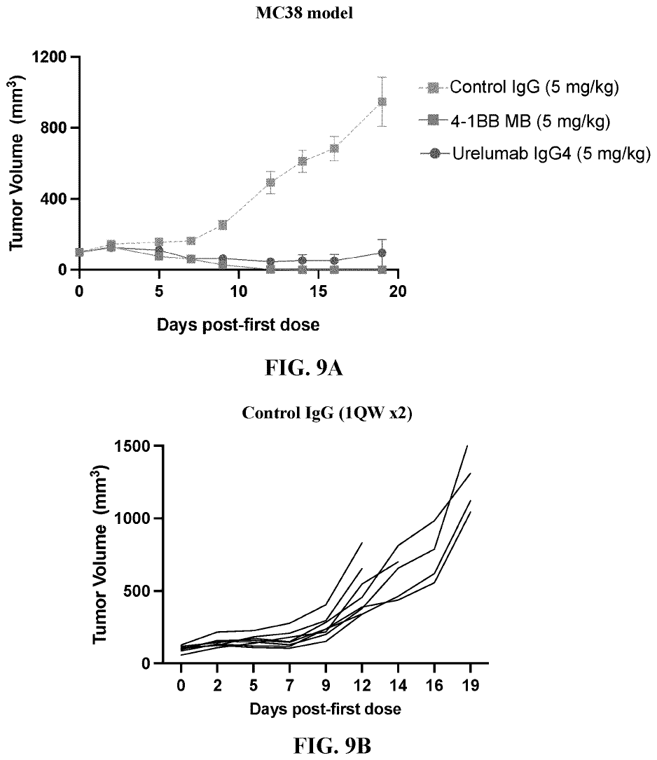

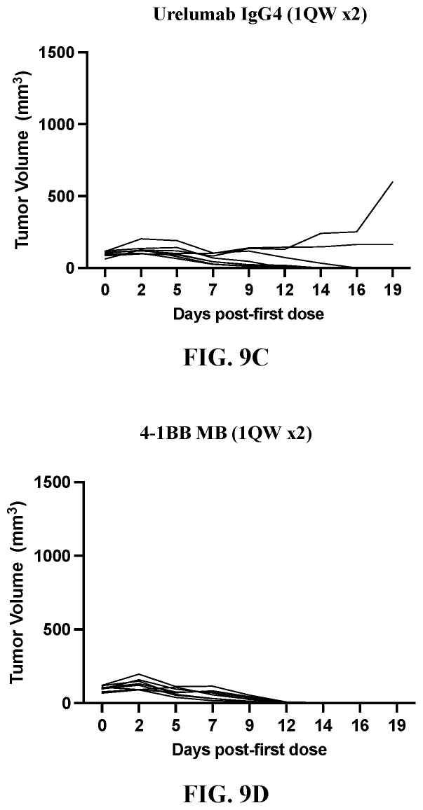

α-4-1BB MBs demonstrated superior efficacy compared to urelumab. Both the urelumab IgG4 and the α-4-1BB MB treatment groups exhibited reductions in tumor volume compared to the Control IgG group (FIG. 9A). The α-4-1BB MB demonstrated superior in vivo efficacy as compared to urelumab IgG4. 100% (8 out of 8) of the mice treated with α-4-1BB MB showed complete tumor regression, whereas only 75% (6 out of 8) of the mice treated with urelumab showed complete tumor regression (FIG. 9D).

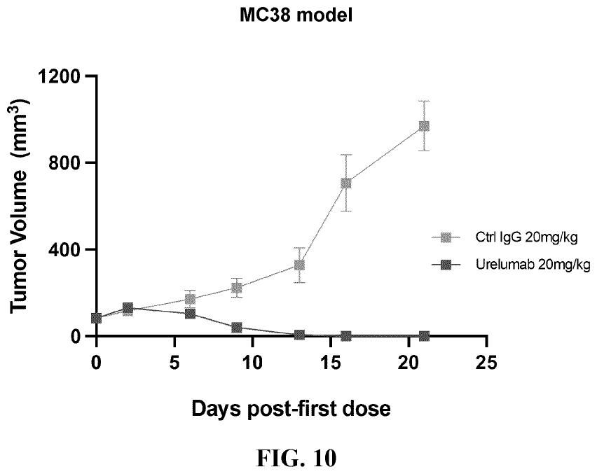

These results—with 100% of mice exhibiting complete tumor regression—were obtained with α-4-1BB MB at a 5 mg/kg dose. To obtain the equivalent efficacy, uurelumab IgG4 had to be administered at a dose as high as 20 mg/kg (FIG. 10).

### Example 13. Safety of Urelumab IgG and α-4-1BB MBs in a Xenograft Mouse Model

The safety of exemplary MBs and urelumab IgG4 at a therapeutically-equivalent dose was evaluated in a colorectal cancer (CRC) Xenograft model.

Toxicity studies were conducted on C57BL/6 mice with a human 4-1BB/4-1BBL double knock-in “(Hu4-1BB KI mice”). Mice were treated with α-4-1BB MB, urelumab, or control IgG4 once per week for 3 weeks by intraperitoneal (IP) injection. Throughout the study, non-terminal blood was sampled, and body weight was measured 3 times per week. The study reached endpoint at 1 week post last dose (Day 21). At endpoint, liver and terminal blood were harvested. Liver tissue was subjected to histopathology (Formalin-Fixed, Paraffin-Embedded (FFPE_preparation). Liver enzymes were quantified in endpoint serum isolated from terminal blood. PK was assessed in isolated serum from non-terminal blood samples.

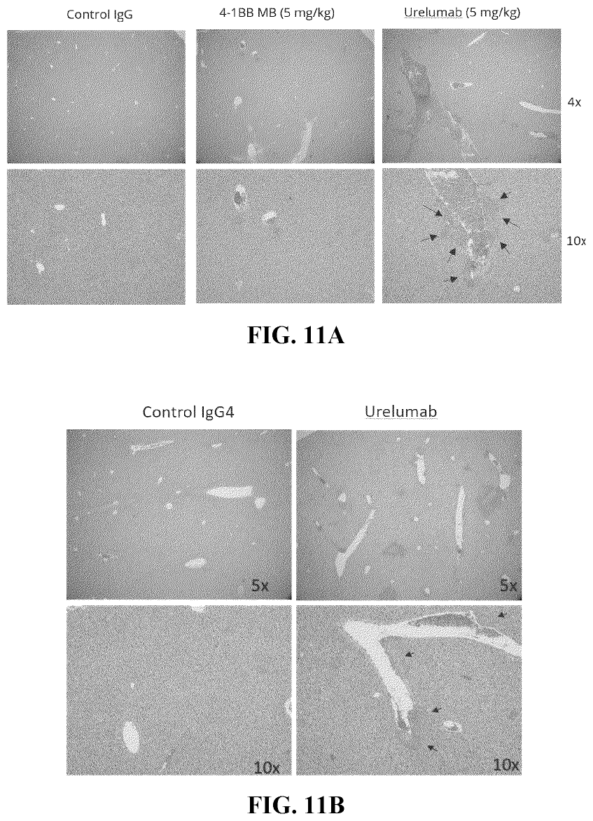

The α-4-1BB MB demonstrated a superior liver safety profile compared to urelumab at the same dosage (5 mg/kg), as demonstrated by the significant increase in liver inflammation induced by urelumab (FIGS. 11A and 11B; arrows indicate regions of inflammation). The therapeutically equivalent dosage of urelumab (at 20 mg/kg) also exhibited severe liver inflammation (FIG. 11B), indicating that urelumab has a narrow therapeutic index in contrast to 4-1-BB MBs.

### Example 14. Pharmacokinetics of α-4-1BB IgG and MB in Mouse Serum

This Example describes experiments to determine the pharmacokinetics of α-4-1BB IgG and α-4-1BB MBs.

Serum was isolated from non-terminal blood that was sampled from mice 3 times per week (1, 3, 7, 8, 10, 15 and 20 days post first dose). Serum concentrations of MB/IgG were measured by ELISA. ELISA plates were coated with Goat F(ab′)₂ α-human Kappa (or Lambda) overnight. The next day, plates were washed, blocked, and incubated with serum samples from each mouse and timepoint, along with a series of known concentration of α-4-1BB IgGs to generate a standard curve. The plates were then washed and incubated with a Goat Anti-Human Fcy-HRP secondary antibody to detect bound MB/IgG from the serum samples. The plates were washed again and developed with TMB substrate to detect HRP and read at OD 450 nm. Serum drug concentrations were calculated by interpolating against the standard curve (OD 450 nm vs. standard concentration).

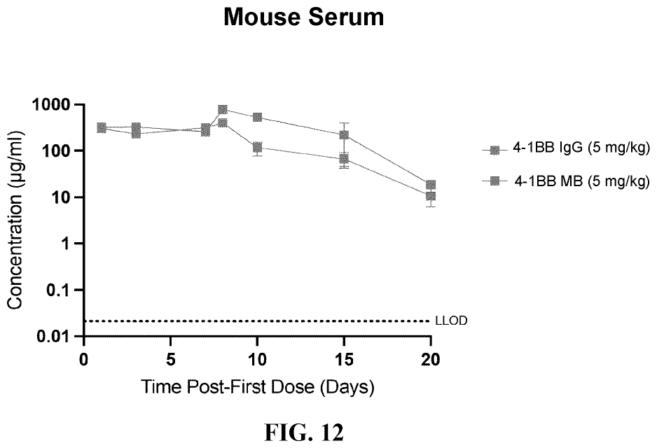

FIG. 12 depicts the concentrations of α-4-1BB IgG and α-4-1BB MB detected in the mice sera throughout the study. Both the IgG and the MB were detected at high concentrations, which demonstrates that the 4-1BB MB has excellent exposure comparable to parental IgG in immune competent mice.

### SEQUENCE LISTING

Underlining within fusion sequences indicate linker sequences.

Bolding within fusion sequences indicate ferritin or ferritin subunit sequences.

Within variable region sequences, underlining and bolding together indicate complementary determining regions sequences.

Boxed and bolded residues indicate residues that are mutated relative to a reference molecule, e.g. relative to an IgG1 Fc.

hFTL
SEQ ID NO: 1
MSSQIRQNYSTDVEAAVNSLVNLYLQASYTYLSLGFYFDRDDVALEGVSHFFRELA
EEKREGYERLLKMQNQRGGRALFQDIKKPAEDEWGKTPDAMKAAMALEKKLNQALLDLHALG
SARTDPHLCDFLETHFLDEEVKLIKKMGDHLTNLHRLGGPEAGLGEYLFERLTLRHD
N_hFTL
SEQ ID NO: 2
SSQIRQNYSTDVEAAVNSLVNLYLQASYTYLSLGFYFDRDDVALEGVSHFFRELAE
EKREGYERLLKMQNQRGGRALFQDIKKPAEDEW
C_hFTL
SEQ ID NO: 3
GKTPDAMKAAMALEKKLNQALLDLHALGSARTDPHLCDFLETHFLDEEVKLIKKMG
DHLTNLHRLGGPEAGLGEYLFERLTLRHD
IgG1 Fc
SEQ ID NO: 4
DKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
IgG1 scFc
SEQ ID NO: 5
DKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGKGGGGSGGGGSGGGGS
GGGGSGGGGSGGGGSGGGGSGGGGSGGGGGGGGSGGGGSGGGGSGGGGSGGGGSDKTHTCP
PCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGK
Example GS linker
SEQ ID NO: 6
GGGS
Example GS linker
SEQ ID NO: 7
GGGGS
SEQ ID NOs: 8-596 as shown in Tables 1A-3C.
scFc-C_hFTL IgG1 WT
SEQ ID NO: 597
DKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGKGGGGSGGGGSGGGGS
GGGGSGGGGSGGGGSGGGGSGGGGSGGGGSGGGGSGGGGSGGGGSGGGGSGGGGSDKTHTCP
PCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGKGGGGSGGGGSGGGGSGGGGSGGGGSGGG
KTPDAMKAAMALEKKLNQALLDLHALGSARTDPHLCDFLETHFLDEEVKLIKKMGDHLTNLH
RLGGPEAGLGEYLFERLTLRHD
98scFc-C_hFTL IgGI LLRAL
SEQ ID NO: 598

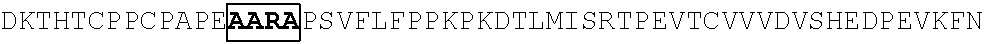

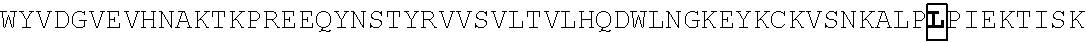

AKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGKGGGGSGGGGSGGGGS
GGGGGGGGSGGGGSGGGGSGGGGSGGGGSGGGGSGGGGSGGGGSGGGGSGGGGSDKTHTCP

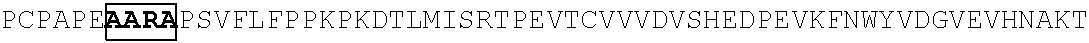

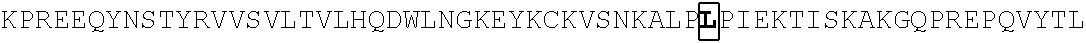

PPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGKGGGGSGGGGSGGGGSGGGGSGGGGSGGG
KTPDAMKAAMALEKKLNQALLDLHALGSARTDPHLCDFLETHFLDEEVKLIKKMGDHLTNLH
RLGGPEAGLGEYLFERLTLRHD
human IgG4 heavy chain constant region sequence
SEQ ID NO: 599
ASTKGPSVFPLAPCSRSTSESTAALGCLVKDYFPEPVTVSWNSGALTSGVHTFPAV
LQSSGLYSLSSVVTVPSSSLGTKTYTCNVDHKPSNTKVDKRVESKYGPPCPSCPAPEFLGGP
SVFLFPPKPKDTLMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYTLPPSQEEMTKNQ
VSLTCLVKGFYPSDIAVEWESNGQPENNYKTTPPVLDSDGSFFLYSRLTVDKSRWQEGNVES
CSVMHEALHNHYTQKSLSLSLGK
human IgGI heavy chain constant region sequence
SEQ ID NO: 600
ASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPELL
GGPSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLYSKLTVDKSRWQQGN
VFSCSVMHEALHNHYTQKSLSLSPGK
human IgG2 heavy chain constant region sequence
SEQ ID NO: 601
ASTKGPSVFPLAPCSRSTSESTAALGCLVKDYFPEPVTVSWNSGALTSGVHTFPAV
LQSSGLYSLSSVVTVPSSNFGTQTYTCNVDHKPSNTKVDKTVERKCCVECPPCPAPPVAGPS
VFLFPPKPKDTLMISRTPEVTCVVVDVSHEDPEVQFNWYVDGVEVHNAKTKPREEQFNSTER
VVSVLTVVHQDWLNGKEYKCKVSNKGLPAPIEKTISKTKGQPREPQVYTLPPSREEMTKNQV
SLTCLVKGFYPSDISVEWESNGQPENNYKTTPPMLDSDGSFFLYSKLTVDKSRWQQGNVFSC
SVMHEALHNHYTQKSLSLSPGK
human IgG3 heavy chain constant region sequence
SEQ ID NO: 602
ASTKGPSVFPLAPCSRSTSGGTAALGCLVKDYFPEPVTVSWNSGALTSGVHTFPAV
LQSSGLYSLSSVVTVPSSSLGTQTYTCNVNHKPSNTKVDKRVELKTPLGDTTHTCPRCPEPK
SCDTPPPCPRCPEPKSCDTPPPCPRCPEPKSCDTPPPCPRCPAPELLGGPSVFLFPPKPKDT
LMISRTPEVTCVVVDVSHEDPEVQFKWYVDGVEVHNAKTKPREEQYNSTFRVVSVLTVLHQD
WLNGKEYKCKVSNKALPAPIEKTISKTKGQPREPQVYTLPPSREEMTKNQVSLTCLVKGFYP
SDIAVEWESSGQPENNYNTTPPMLDSDGSFFLYSKLTVDKSRWQQGNIFSCSVMHEALHNRF
TQKSLSLSPGK
human Igκ light chain constant region sequence
SEQ ID NO: 603
RTVAAPSVFIFPPSDEQLKSGTASVVCLLNNFYPREAKVQWKVDNALQSGNSQESV
TEQDSKDSTYSLSSTLTLSKADYEKHKVYACEVTHQGLSSPVTKSENRGEC
human Igλ1 light chain constant region sequence
SEQ ID NO: 604
GQPKANPTVTLFPPSSEELQANKATLVCLISDFYPGAVTVAWKADGSPVKAGVETT
KPSKQSNNKYAASSYLSLTPEQWKSHRSYSCQVTHEGSTVEKTVAPTECS
human Igλ2 light chain constant region sequence
SEQ ID NO: 605
GQPKAAPSVTLFPPSSEELQANKATLVCLISDFYPGAVTVAWKADSSPVKAGVETT
TPSKQSNNKYAASSYLSLTPEQWKSHRSYSCQVTHEGSTVEKTVAPTECS
human Igλ3 light chain constant region sequence
SEQ ID NO: 606
GQPKAAPSVTLFPPSSEELQANKATLVCLISDFYPGAVTVAWKADSSPVKAGVETT
TPSKQSNNKYAASSYLSLTPEQWKSHKSYSCQVTHEGSTVEKTVAPTECS
human Igλ6 light chain constant region sequence
SEQ ID NO: 607
GQPKAAPSVTLFPPSSEELQANKATLVCLISDFYPGAVKVAWKADGSPVNTGVETT
TPSKQSNNKYAASSYLSLTPEQWKSHRSYSCQVTHEGSTVEKTVAPAECS
human Igλ7 light chain constant region sequence
SEQ ID NO: 608
GQPKAAPSVTLFPPSSEELQANKATLVCLVSDENPGAVTVAWKADGSPVKVGVETT
KPSKQSNNKYAASSYLSLTPEQWKSHRSYSCRVTHEGSTVEKTVAPAECS

### EQUIVALENTS/OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features herein before set forth.

1. A fusion polypeptide comprising: (1) a tumor necrosis factor (TNF) receptor-binding moiety and (2) a nanocage monomer or subunit thereof, wherein the TNF receptor-binding moiety is not capable of binding to death receptor 4 (DR4) or death receptor 5 (DR5).

2. The fusion polypeptide of claim 1, wherein the TNF receptor-binding moiety comprises an antibody or antigen-binding fragment thereof.

3. The fusion polypeptide of claim 2, wherein the antibody or antigen-binding fragment thereof comprises an immunoglobulin heavy chain variable region (V_{H}) and an immunoglobulin light chain variable region (V_{L} or V_{K}).

4. The fusion polypeptide of claim 3, wherein the antibody or antigen-binding fragment thereof comprises a Fab fragment.

5. The fusion polypeptide of claim 4, wherein the Fab fragment is a single-chain Fab fragment (scFab).

6. The fusion polypeptide of any one of claims 1-5, wherein the TNF receptor-binding moiety is not capable of binding to a TNF receptor which comprises a cytoplasmic tail which comprises a death domain.

7. The fusion polypeptide of any one of claims 1-6, wherein the TNF-receptor-binding moiety is capable of binding to a TNF receptor that is expressed on a hematopoietic cell.

8. The fusion polypeptide of claim 7, wherein the TNF receptor is selected from the group consisting of 4-1BB (also known as CD137), OX40 (also known as CD134), CD40, herpes-virus entry mediator (HVEM), CD30, CD27 (also known as TNFRSF7), GITR (also known as TNFRSF18), and TNFR2.

9. The fusion polypeptide of claim 8, wherein the TNF receptor is 4-1BB or OX40.

10. The fusion polypeptide of claim 9, wherein the TNF receptor-binding moiety comprises an antibody fragment comprising a heavy chain variable region (V_{H}) and light chain variable region (V_{L} or V_{K}), wherein the CDRs of the V_{H} and the V_{L} or V_{K} (CDR-H1, CDR-H2, CDR-H3, CDR-L1, CDR-L2, and CDR-L3) have sequences which each differ by at most two amino acids from the CDRs of a 4-1BB or OX40 antibody.

11. The fusion polypeptide of claim 10, wherein the CDRs of the V_{H} and the V_{L} or V_{K} have sequences which are identical to those of the heavy and light chain CDRs of a 4-1BB or OX40 antibody, except for one or two amino acid substitutions total across all six CDRs.

12. The fusion polypeptide of claim 11, wherein the CDRs of the V_{H} and the V_{L} or V_{K} are identical to those of the heavy and light chain CDRs of a 4-1BB or OX40 antibody.

13. The fusion polypeptide of claim 9, wherein the TNF receptor-binding moiety comprises an antibody fragment comprising heavy and light chain variable regions with sequences that have at least 85% identity to those of the heavy and light chain variable regions of a 4-1BB or OX40 antibody.

14. The fusion polypeptide of any one of claims 1-13, wherein the nanocage monomer is a ferritin monomer or a subunit thereof.

15. The fusion polypeptide of claim 14, wherein the ferritin monomer is a human ferritin monomer.

16. The fusion polypeptide of claim 14 or 15, wherein the ferritin monomer is a ferritin light chain.

17. The fusion polypeptide of any one of claims 1-16, wherein the TNF receptor-binding moiety is linked via an amino acid linker to the nanocage monomer or subunit thereof.

18. The fusion polypeptide of any one of claims 1-17, wherein the TNF receptor-binding moiety is linked via the N-terminus of the nanocage monomer or subunit thereof.

19. A self-assembled polypeptide complex comprising:
(a) a plurality of TNF receptor-binding fusion polypeptides, each TNF receptor-binding fusion polypeptide being a fusion polypeptide of any one of claims 1-18; and
(b) a plurality of Fc fusion polypeptides, each Fc fusion polypeptide comprising (1) an Fc polypeptide and (2) a nanocage monomer or subunit thereof.

20. The self-assembled polypeptide complex of claim 19, further comprising
(c) a plurality of tumor-binding fusion polypeptides, each tumor-binding fusion polypeptide comprising (1) tumor-binding moiety and (2) a nanocage monomer or subunit thereof.

21. The self-assembled polypeptide complex of claim 19 or 20, wherein, within each
Fc fusion polypeptide, the Fc polypeptide is linked via an amino acid linker to the nanocage monomer or subunit thereof.

22. The self-assembled polypeptide complex of claim 21, wherein the Fc polypeptide is linked via the N-terminus of the nanocage monomer or subunit thereof.

23. The self-assembled polypeptide complex of claim 22, wherein the Fc polypeptide is linked via the C-terminus of the nanocage monomer or subunit thereof.

24. The self-assembled polypeptide complex of any one of claims 20-23, wherein, with
each tumor-binding fusion polypeptide, the tumor-binding moiety is linked via an amino acid linker to the nanocage monomer or subunit thereof.

25. The self-assembled polypeptide complex of claim 24, wherein the tumor-binding moiety is linked via the N-terminus of the nanocage monomer or subunit thereof.

26. The self-assembled polypeptide complex of any one of claims 20-25, wherein the tumor-binding moiety comprises an antibody or antigen-binding fragment thereof.

27. The self-assembled polypeptide complex of claim 26, wherein the antibody or antigen-binding fragment thereof comprises an immunoglobulin heavy chain variable region (V_{H}) and an immunoglobulin light chain variable region (V_{L} or V_{K}).

28. The self-assembled polypeptide complex of claim 27, wherein the antibody or antigen-binding fragment thereof comprises a Fab fragment.

29. The self-assembled polypeptide complex of claim 28, wherein the Fab fragment is a single-chain Fab fragment (scFab).

30. The self-assembled polypeptide complex of any one of claims 26-29, wherein the tumor-binding moiety is a PD-L1-binding moiety, an EGFR-binding moiety, or a B7-H3-binding moiety.

31. The self-assembled polypeptide complex of claim 30, wherein the tumor-binding moiety comprises an antibody fragment comprising a heavy chain variable region (V_{H}) and light chain variable region (V_{L} or V_{K}),
wherein the CDRs of the V_{H} and the V_{L} or V_{K} (CDR-H1, CDR-H2, CDR-H3, CDR-L1, CDR-L2, and CDR-L3) have sequences which each differ by at most two amino acids from the CDRs of a PD-L1 binding moiety, an EGFR-binding moiety, or a B7-H3-binding moiety antibody.

32. The self-assembled polypeptide complex of claim 31, wherein the CDRs of the V_{H} and the V_{L} or V_{K} have sequences which are identical to those of the heavy and light chain CDRs of a PD-L1, an EGFR-binding moiety, or a B7-H3-binding moiety antibody, except for one or two amino acid substitutions total across all six CDRs.

33. The self-assembled polypeptide complex of claim 32, wherein the CDRs of the V_{H} and the V_{L} or V_{K} are identical to those of the heavy and light chain CDRs of a PD-L1, EGFR, or B7-H3 antibody.

34. The self-assembled polypeptide complex of claim 30, wherein the tumor-binding moiety comprises an antibody fragment comprising heavy and light chain variable regions with sequences that have at least 85% identity to those of the heavy and light chain variable regions of a PD-L1, EGFR, or B7-H3 antibody.

35. The self-assembled polypeptide complex of any one of claims 19-34, wherein the nanocage monomers within the Fc fusion polypeptide and within TNF receptor-binding fusion polypeptide and/or the tumor-binding fusion polypeptide are each a ferritin monomer or a subunit thereof.

36. The self-assembled polypeptide complex of claim 35, wherein the ferritin monomer is a human ferritin monomer.

37. The self-assembled polypeptide complex of claim 35 or 36, wherein the ferritin monomer is a ferritin light chain.

38. The self-assembled polypeptide complex of claim 37, which does not comprise any ferritin heavy chains or subunits of ferritin heavy chains.

39. The self-assembled polypeptide complex of any one of claims 35-38, which does not comprise any iron-binding moieties.

40. A pharmaceutical composition comprising a self-assembled polypeptide complex of any one of claims 19-39 and a pharmaceutically acceptable excipient.

41. A method of treating, ameliorating, or preventing a disease or condition, the method comprising administering to a subject the self-assembled polypeptide complex of any of claims 19-39 or the pharmaceutical composition of claim 40.

42. The method of claim 41, wherein the subject is a mammal.

43. The method of claim 42, wherein the subject is human.

44. The method of claim 43, wherein the disease or condition is cancer.

45. The method of claim 44, wherein the subject has an immunologically cold tumor.

46. The method of any one of claims 41-45, wherein the subject has a tumor and said step of administering results in regression of the tumor.

47. The method of claim 46, wherein said step of administering results in complete regression of the tumor.

48. Use of the self-assembled polypeptide complex of any one of claims 19-39 or the pharmaceutical composition of claim 40 to treat, ameliorate, or prevent a disease or condition in a subject.

49. The use of claim 48, wherein the subject is a mammal.

50. The use of claim 49, wherein the subject is human.

51. The use of any one of claims 48-50, wherein the disease or condition is cancer.

52. The use of claim 51, wherein the cancer is a solid cancer.

53. The use of claim 51, wherein the cancer is a hematological cancer.

54. The use of any one of claims 48-50, wherein the disease or condition is an autoimmune disease or condition.
